(19) United States
(12) Patent Application Publication
(10) Pub. No.: US 2026/0125340 A1
(43) Pub. Date: May 7, 2026

(54) METHOD FOR PREPARING AMIDES FROM ESTERS

(71) Applicant: BASF SE, Ludwigshafen (DE)

(72) Inventors: Thomas Schaub, Ludwigshafen (DE); Christian Harald Winter, Ludwigshafen (DE); Stefan Benson, Ludwigshafen (DE); Roland Goetz, Ludwigshafen (DE); Desislava Slavcheva Petkova, Ludwigshafen (DE); A. Stephen K. Hashmi, Heidelberg (DE); Nadja Elena Niggli, Heidelberg (DE)

(21) Appl. No.: 19/117,432

(22) PCT Filed: Oct. 5, 2023
(86) PCT No.: PCT/EP2023/077628

§ 371 Date: Apr. 1, 2025

(30) Foreign Application Priority Data
Oct. 6, 2022 (EP) 22200088.7

(51) Int. Cl. C07C 231/02; C07C 253/30; C07D 209/12; C07D 213/75; C07D 215/40; C07D 231/14

(52) U.S. Cl. CPC C07C 231/02; C07C 253/30; C07D 209/12; C07D 213/75; C07D 215/40; C07D 231/14

(57) ABSTRACT

The present invention relates to a method for preparing of an amide of the formula (I-1) or a diamide of the formula (I-2) where the variables areas defined in the claims and the description, by reacting an ester with an aromatic or heteroaromatic amine in the presence of an alkali metal-containing base and a Lewis acid; where the reaction is carried out under essentially anhydrous conditions.

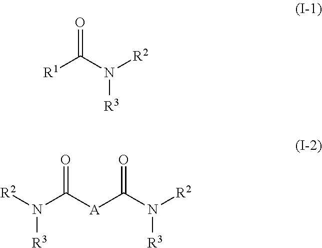

The present invention relates to a method for preparing of an amide of the formula (I-1) or a diamide of the formula (I-2) as defined below derived from an aromatic or heteroaromatic amine by reacting an ester with an aromatic or heteroaromatic amine in the presence of an alkali metal-containing base and a Lewis acid; where the reaction is carried out under essentially anhydrous conditions.

### TECHNICAL BACKGROUND

Amide functional groups are ubiquitous in biological, pharmaceutical, agrochemical and natural products. Efficient synthesis routes to amides are therefore of high importance.

Aromatic amides are of great interest especially as pharmaceuticals and pesticides and as precursors of such active ingredients. The fungicides boscalid, fluxapyroxad or bixafen are prominent representatives, to name just a few examples. They are generally synthesized from the corresponding acid chlorides and amines in combination with stochiometric amounts of a base, such as triethylamine, to form the corresponding amide bond in these target molecules in sufficiently high yields (see for example Green Chemistry, 2021, 23, 8169-8180). The acid chlorides are usually formed from the corresponding acid with a chlorination agent, such as thionylchloride, whereas the acid is usually made by hydrolysis of the ester (esters are more common synthetic intermediates than the corresponding acids). A drawback of this route to the amide is the three steps generally required—ester hydrolysis, acid chloride formation, amidation—, the use of chlorinating agents and the amount of waste formed. Other established routes to amides involve the reaction of the corresponding acid with an amine using an activating agent, such as HATU or EDC. The drawbacks of this route are similar to those mentioned above—two steps required and the formation of stoichiometric amounts of waste byproducts.

Therefore, synthetic routes to amides via the direct reaction of esters with amines without the use of halogenating agents or other stoichiometric activating agents and in less steps is desirable. Moreover, there is a special need for efficient amidation routes starting from (hetero)aromatic amines, which, given their weaker nucleophility as compared to aliphatic amines, is still challenging. As explained above, (hetero)aromatic amide moieties are widespread in many pharmaceuticals and pesticides.

During the last years, several synthetic methods for preparing amides via direct reaction of esters with (aromatic) amines have been suggested.

H. Moromoto et al. describe in Organic Letters, 2014, 16, 2018-2021 the La(OTf)₃ (OTf=CF₃SO₃) catalyzed amidation of esters with different amines. For aromatic amines, catalyst loadings of 2-5 mol-% of the lanthanum catalyst are necessary. Moreover, the amidation was only carried out with very electron-rich and activated aromatic amines, such as 4-methoxyaniline. This route is thus viable for a limited substrate scope only. Moreover, lanthanum catalysts are expensive, and their production, given that lanthanum is a rare earth metal, involves polluting and hazardous processes.

B. D. Mkhonazi et al. describe in Molecules, 2020, 25, 1040-1048 Lewis acid (e.g. FeCl₃, FeBr₃, AlCl₃, BiCl₃) catalyzed amidations of esters with different amines. For aromatic amines, catalyst loadings of 15 mol % of FeCl₃ were necessary and the amidation with aromatic amines worked only if ethyl 2-pyridine-carboxylate was used as ester, the 2-pyridyl group being essential for the activation of the catalyst. Also this route is thus viable for a limited substrate scope only.

T. B. Halima et al. describe in Angewandte Chemie, International Edition, 2018, 57, 12925-12929 the direct amidation of esters with aromatic amines by using Ni(COD)₂ (COD=1,5-cyclooctadiene) in combination with the N-heterocyclic carbene IPr (1,3-bis(2,6-diisopropylphenyl)imidazol-2-ylidene) as the catalyst. Optimal results were reported at a loading of 10 mol % of the Ni complex with 20 mol % of the N-heterocyclic carbene. The system works for a variety of anilines. A drawback of this system is the use of relative large amounts of the sensitive and expensive Ni(COD)₂ as well as the N-heterocyclic carbene (NHC) ligand.

W. I. Nicholson et al. describe in Angewandte Chemie, International Edition 2021, 60, 21686-21874 the direct amidation of esters with aromatic amines by using ball mill technology in the presence of an alkoxide base like KOtBu (potassium tert-butanolate) as a mediator. The system works for a variety of different anilines. Unfortunately, high yields can only be obtained when stochiometric amounts of the alkoxide base are used. Moreover, ball milling equipment is not universally available and the technology is so far not applicable in organic syntheses on industrial scale.

R. Zhang et al. describe in Green Chemistry 2021, 23, 3972-3982 the solvent-free direct amidation of esters with aromatic amines using an alkoxide base like NaOtBu (sodium tert-butanolate) as a mediator. The system works for a variety of different electron-rich as well as electron-poor anilines. Unfortunately, high yields can only be obtained when over-stochiometric amounts of the alkoxide base are used.

Z. Fu et al. describe in Journal of Organic Chemistry, 2021, 86, 2339-2358 the direct amidation of esters with aromatic amines by using a N-heterocyclic carbene (NHC) ligand-containing manganese(I) pincer complex as catalyst with an alkoxide base as co-catalyst. Optimal results were reported at a loading of 1 mol % of the Mn complex with 20 mol % of the alkoxide base NaOtBu (sodium tert-butanolate). The system works for a variety of different anilines. A drawback of this system is the use of relatively large amounts of the alkoxide base of 20 mol % as well as the complex Mn catalyst, the production of which requires a multistep synthesis.

It was the object of the present invention to provide a process for the synthesis of amides from esters and aromatic or heteroaromatic amines which works for a broad substrate scope and uses a simple and economic catalyst system which works also at low catalyst loadings.

These objects are achieved by the combined use of a Lewis acid catalyst and an alkali metal-containing base under essentially anhydrous conditions.

### SUMMARY OF THE INVENTION

The present invention relates thus to a method for preparing an amide of the formula (I-1) or a diamide of the formula (I-2)

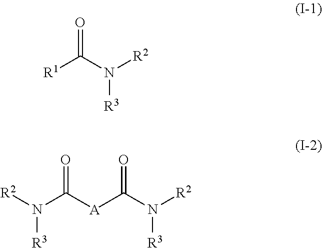

where
R¹ is selected from the group consisting of hydrogen, C₁-C₃₀-alkyl which is unsubstituted or carries m radicals R^{a}, C₁-C₃₀-haloalkyl which is unsubstituted or carries m radicals R^{a}, C₂-C₃₀-alkenyl which is unsubstituted or carries m radicals R^{a}, C₂-C₃₀-haloalkenyl which is unsubstituted or carries m radicals R^{a}, C₂-C₃₀-alkynyl which is unsubstituted or carries m radicals R^{a}, C₂-C₃₀-haloalkynyl which is unsubstituted or carries m radicals R^{a}, C₃-C₃₀-cycloalkyl which is unsubstituted or carries m radicals R^{b}, C₆-C₂₂-aryl which is unsubstituted or carries m radicals R^{b}, and a 3- to 30-membered saturated, partially unsaturated or maximally unsaturated heterocyclic ring containing 1, 2, 3 or 4 heteroatoms or heteroatom groups selected from, N, O, S, SO and SO₂ as ring members, which is unsubstituted or carries m radicals R^{b};
R² is C₆-C₂₂-aryl which is unsubstituted or carries m radicals R^{b}, or is a 5- to 30-membered heteroaromatic ring containing 1, 2, 3 or 4 heteroatoms selected from N, O and S as ring members, where the heteroaromatic ring is unsubstituted or carries m radicals R^{b}; R³ is selected from the group consisting of hydrogen, C₁-C₃₀-alkyl which is unsubstituted or carries m radicals R^{a}, C₁-C₃₀-haloalkyl which is unsubstituted or carries m radicals R^{a}, C₂-C₃₀-alkenyl which is unsubstituted or carries m radicals R^{a}, C₂-C₃₀-haloalkenyl which is unsubstituted or carries m radicals R^{a}, C₂-C₃₀-alkynyl which is unsubstituted or carries m radicals R^{a}, C₂-C₃₀-haloalkynyl which is unsubstituted or carries m radicals R^{a}, C₃-C₃₀-cycloalkyl which is unsubstituted or carries m radicals R^{b}, C₆-C₂₂-aryl which is unsubstituted or carries m radicals R^{b}, and a 3- to 30-membered saturated, partially unsaturated or maximally unsaturated heterocyclic ring containing 1, 2, 3 or 4 heteroatoms or heteroatom groups selected from, N, O, S, SO and SO₂ as ring members, which is unsubstituted or carries m radicals R^{b};
or
R³ forms a saturated or unsaturated 2-, 3- or 4-membered linking group to a carbon or nitrogen ring atom of the aryl or heteroaromatic ring R²; where the linking group may comprise 1 or 2 heteroatoms or heteroatom groups selected from, N, O, S, SO and SO₂; where the linking group may carry 1, 2 or 3 radicals selected from the group consisting of halogen, cyano, C₁-C₄-alkyl, C₁-C₄-haloalkyl, C₁-C₄-alkoxy and C₁-C₄-haloalkoxy;
A is a divalent aliphatic, aliphatic-cycloaliphatic, cycloaliphatic, aromatic, aromatic-aliphatic or heterocyclic moiety;
each R^{a} is independently selected from the group consisting of cyano, nitro, hydroxyl, C₁-C₄-alkoxy, C₁-C₄-haloalkoxy, C(═O)R^{c}, amino, C₁-C₄-alkylamino, di-(C₁-C₄-alkyl)-amino, C₃-C₂₀-cycloalkyl, C₆-C₂₂-aryl which is unsubstituted or carries m radicals R^{d}, and a 3- to 20-membered saturated, partially unsaturated or maximally unsaturated heterocyclic ring containing 1, 2, 3 or 4 heteroatoms or heteroatom groups selected from, N, O, S, SO and SO₂ as ring members, which is unsubstituted or carries m radicals R^{d};
each R^{b} is independently selected from the group consisting of halogen, cyano, nitro, hydroxyl, C₁-C₄-alkoxy, C₁-C₄-haloalkoxy, amino, C₁-C₄-alkylamino, di-(C₁-C₄-alkyl)-amino, NR^{e}R^{f}, C(═O)NR^{e}R^{f}, C₁-C₂₀-alkyl, C₁-C₂₀-haloalkyl, C₂-C₂₀-alkenyl, C₂-C₂₀-haloalkenyl, C₂-C₂₀-alkynyl, C₂-C₂₀-haloalkynyl, C₃-C₂₀-cycloalkyl, C₆-C₂₂-aryl which is unsubstituted or carries m radicals R^{d}, and a 3- to 20-membered saturated, partially unsaturated or maximally unsaturated heterocyclic ring containing 1, 2, 3 or 4 heteroatoms or heteroatom groups selected from, N, O, S, SO and SO₂ as ring members, which is unsubstituted or carries m radicals R^{d};
each R^{c} is independently selected from the group consisting of C₁-C₄-alkyl, C₁-C₄-haloalkyl, C₁-C₄-alkyl which carries a group NR^{e}R^{f}, C₁-C₄-alkoxy and C₁-C₄-haloalkoxy;
each R^{d} is independently selected from the group consisting of halogen, cyano, hydroxyl, C₁-C₄-alkyl, C₁-C₄-haloalkyl, C₁-C₄-alkoxy and C₁-C₄-haloalkoxy;
each R^{e} is independently selected from the group consisting of hydrogen and C₁-C₄-alkyl;
each R^{f} is independently selected from the group consisting of —C(═O)-phenyl and phenyl which is unsubstituted or substituted by 1, 2 or 3 radicals selected from the group consisting of halogen, cyano, C₁-C₄-alkyl, C₁-C₄-haloalkyl, C₁-C₄-alkoxy and C₁-C₄-haloalkoxy;
each m is independently 1, 2, 3, 4 or 5;
which method comprises reacting an ester compound (II) of the formula (II-1) or (II-2)

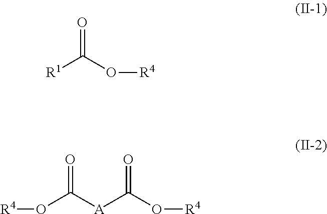

wherein
R¹ and A are as defined above; and
R⁴ is selected from the group consisting of C₁-C₃₀-alkyl, C₆-C₁₄-aryl and C₆-C₁₄-aryl-C₁-C₄-alkyl;
with an amine of the formula (III)

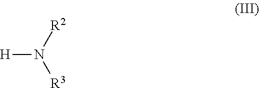

wherein R² and R³ are as defined above,
in the presence of an alkali metal-containing base and a Lewis acid;
where the reaction is carried out under anhydrous conditions, where the water content in the reaction mixture is at most 0.15% by weight, relative to the total weight of the reaction mixture.

### DETAILED DESCRIPTION OF THE INVENTION

### Definitions

In the present description, the term radical is used interchangeably with the term group or substituent, when defining the variables R^{x} in the presented formulae.

The term “halogen” denotes in each case fluorine, chlorine, bromine or iodine.

The term “alkyl” indicates a saturated straight-chain or branched aliphatic non-cyclic hydrocarbon radical having for example 1 to 30 (“C₁-C₃₀-alkyl”) carbon atoms, or 1 to 20 (“C₁-C₂₀-alkyl”) carbon atoms, or 1 to 10 (“C₁-C₁₀-alkyl”) carbon atoms, or 1 to 6 (“C₁-C₆-alkyl”) or 1 to 4 (“C₁-C₄-alkyl”) or 1 to 3 (“C₁-C₃-alkyl”) or 1 or 2 (“C₁-C₂-alkyl”) carbon atoms. C₁-C₂-Alkyl is methyl or ethyl. Examples for C₁-C₃-alkyl are, in addition to those mentioned for C₁-C₂-alkyl, propyl and isopropyl. Examples for C₁-C₄-alkyl are, in addition to those mentioned for C₁-C₃-alkyl, butyl, 1-methylpropyl (sec-butyl), 2-methylpropyl (isobutyl) or 1,1-dimethylethyl (tert-butyl). Examples for C₁-C₆-alkyl are, in addition to those mentioned for C₁-C₄-alkyl, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. Examples for C₁-C₀-alkyl are, in addition to those mentioned for C₁-C₆-alkyl, heptyl, octyl, 2-ethylhexyl, nonyl, decyl and positional isomers thereof. Examples for C₁-C₂₀-alkyl are, in addition to those mentioned for C₁-C₁₀-alkyl, n-undecyl, n-dodecyl, n-tridecyl, n-tetradecyl, n-hexadecyl, n-heptadecyl, n-octadecyl, n-nonadecyl, n-eicosyl and position isomers thereof. Examples for C₁-C₃₀-alkyl are, in addition to those mentioned for C₁-C₂₀-alkyl, n-henicosyl, n-docosy, n-tricosyl, n-tetracosy, n-pentacosyl, n-hexacosyl, n-octacosy, n-nonacosyl, n-triacontyl and position isomers thereof.

The term “haloalkyl” (also expressed as “alkyl which is partially or fully halogenated”) indicates saturated straight-chain or branched aliphatic non-cyclic hydrocarbon radicals having for example 1 to 30 (“C₁-C₃₀-haloalkyl”) carbon atoms, or 1 to 20 (“C₁-C₂₀-haloalkyl”) carbon atoms, or 1 to 10 (“C₁-C₁₀-haloalkyl”) carbon atoms, or (“C₁-C₆-haloalkyl”) or 1 to 4 (“C₁-C₄-haloalkyl”) or 1 or 2 (“C₁-C₂-haloalkyl”)carbon atoms, where some or all of the hydrogen atoms in these groups are replaced by halogen atoms as mentioned above, in particular fluorine, chlorine and/or bromine. “C₁-C₂-Haloalkyl” refers to alkyl groups having 1 or 2 carbon atoms (as mentioned above), where some or all of the hydrogen atoms in these groups are replaced by halogen atoms as mentioned above, in particular fluorine, chlorine and/or bromine. “C₁-C₃-Haloalkyl” refers to straight-chain or branched alkyl groups having 1 to 3 carbon atoms (as mentioned above), where some or all of the hydrogen atoms in these groups are replaced by halogen atoms as mentioned above, in particular fluorine, chlorine and/or bromine. “C₁-C₄-Haloalkyl” refers to straight-chain or branched alkyl groups having 1 to 4 carbon atoms (as mentioned above), where some or all of the hydrogen atoms in these groups are replaced by halogen atoms as mentioned above, in particular fluorine, chlorine and/or bromine. “C₁-C₆-Haloalkyl” refers to straight-chain or branched alkyl groups having 1 to 6 carbon atoms (as mentioned above), where some or all of the hydrogen atoms in these groups are replaced by halogen atoms as mentioned above, in particular fluorine, chlorine and/or bromine. Examples for C₁-C₂-haloalkyl are chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl or pentafluoro-ethyl. Examples for C₁-C₃-haloalkyl are, in addition to those mentioned for C₁-C₂-haloalkyl, 1-fluoropropyl, 2-fluoropropyl, 3-fluoropropyl, 1,1-difluoropropyl, 2,2-difluoropropyl, 1,2-difluoropropyl, 3,3-difluoropropyl, 3,3,3-trifluoropropyl, heptafluoro-propyl, 1,1,1-trifluoroprop-2-yl, 1,1,1,3,3,3-hexafluoroprop-2-yl, heptafluoroprop-2-yl, 3-chloropropyl and the like. Examples for C₁-C₄-haloalkyl are, in addition to those mentioned for C₁-C₃-haloalkyl, 4-chlorobutyl and the like.

Strictly speaking, the term “alkenyl” indicates monounsaturated (i.e. containing one C—C double bond) straight-chain or branched aliphatic non-cyclic hydrocarbon radicals having for example 2 to 30 (“C₂-C₃₀-alkenyl”) carbon atoms, or 2 to 20 (“C₂-C₂₀-alkenyl”) carbon atoms, or 2 to 10 (“C₂-C₁₀-alkenyl”) carbon atoms, or 2 to 6 (“C₂-C₆-alkenyl”) or 2 to 4 (“C₂-C₄-alkenyl”) carbon atoms, where the C—C double bond can be in any position. As used in the present invention, the term encompasses however also “alkapolyenyl” groups, i.e. straight-chain or branched aliphatic non-cyclic hydrocarbon radicals having for example 4 to 30 (“C₄-C₃₀-alkapolyenyl”) carbon atoms, or 4 to 20 (“C₄-C₂₀-alkapolyenyl”) carbon atoms, or 4 to 10 (“C₄-C₁₀-alkapolyenyl”) carbon atoms, and two or more conjugated or isolated, but non-cumulated C—C double bonds. C₂-alkenyl is ethenyl (vinyl). Examples for C₂-C₃-alkenyl in the strict sense (only 1 C—C double bond) are ethenyl, 1-propenyl, 2-propenyl or 1-methylethenyl. Examples for C₂-C₄-alkenyl in the strict sense (only 1 C—C double bond) are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl and 2-methyl-2-propenyl. Examples for C₂-C₆-alkenyl in the strict sense (only 1 C—C double bond) are ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like. Examples for C₂-C₁₀-alkenyl in the strict sense (only 1 C—C double bond) are, in addition to the examples mentioned for C₂-C₆-alkenyl, 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof. Examples for C₂-C₂₀-alkenyl in the strict sense (only 1 C—C double bond) are, in addition to the examples mentioned for C₂-C₁₀-alkenyl, 1-undecenyl, 2-undecenyl, 3-undecenyl, 4-undecenyl, 5-undecenyl, 1-dodecenyl, 2-dodecenyl, 3-dodecenyl, 4-dodecenyl, 5-dodecenyl, 6-dodecenyl, 1-tridecenyl, 2-tridecenyl, 3-tridecenyl, 4-tridecenyl, 5-tridecenyl, 6-tridecenyl, 1-tetradecenyl, 2-tetradecenyl, 3-tetradecenyl, 4-tetradecenyl, 5-tetradecenyl, 6-tetradecenyl, 7-tetradecenyl, 1-pentadecenyl, 2-pentadecenyl, 3-pentadecenyl, 4-pentadecenyl, 5-pentadecenyl, 6-pentadecenyl, 7-pentadecenyl, 1-hexadecenyl, 2-hexadecenyl, 3-hexadecenyl, 4-hexadecenyl, 5-hexadecenyl, 6-hexadecenyl, 7-hexadecenyl, 8-hexadecenyl, 1-heptadecenyl, 2-heptadecenyl, 3-heptadecenyl, 4-heptadecenyl, 5-heptadecenyl, 6-heptadecenyl, 7-heptadecenyl, 8-heptadecenyl, 1-octadecenyl, 2-octadecenyl, 3-octadecenyl, 4-octadecenyl, 5-octadecenyl, 6-octadecenyl, 7-octadecenyl, 8-octadecenyl, 9-octadecenyl, 1-nonadecenyl, 2-nonadecenyl, 3-nonadecenyl, 4-nonadecenyl, 5-nonadecenyl, 6-nonadecenyl, 7-nonadecenyl, 8-nonadecenyl, 9-nonadecenyl, 1-eicosadecenyl, 2-eicosadecenyl, 3-eicosadecenyl, 4-eicosadecenyl, 5-eicosadecenyl, 6-eicosadecenyl, 7-eicosadecenyl, 8-eicosadecenyl, 9-eicosadecenyl, and the positional isomers thereof.

Examples for alkapolyenyl groups are buta-1,3-dien-1-yl, buta-1,3-dien-2-yl, penta-1,3-dien-1-yl, penta-1,3-dien-2-yl, penta-1,3-dien-3-yl, penta-1,3-dien-4-yl, penta-1,3-dien-5-yl, penta-1,4-dien-1-yl, penta-1,4-dien-2-yl, penta-1,4-dien-3-yl, and the like.

The term “haloalkenyl” as used herein, which may also be expressed as “alkenyl which is substituted by halogen”, refers to unsaturated straight-chain or branched aliphatic non-cyclic hydrocarbon radicals having 2 to 30 (“C₂-C₃₀-haloalkenyl”) or 2 to 20 (“C₂-C₂₀-haloalkenyl”) or 2 to 4 (“C₂-C₄-haloalkenyl”) or 2 to 3 (“C₂-C₃-haloalkenyl”) carbon atoms and one or more double bonds in any position (provided they are not cumulated), where some or all of the hydrogen atoms in these groups are replaced by halogen atoms as mentioned above, in particular fluorine, chlorine and bromine, for example chlorovinyl, chloroallyl and the like.

The term “alkynyl” as used herein indicates straight-chain or branched aliphatic non-cyclic hydrocarbon radicals having for example 2 to 30 (“C₂-C₃₀-alkynyl”) carbon atoms, or 2 to 20 (“C₂-C₂₀-alkynyl”) carbon atoms, or 2 to 10 (“C₂-C₁₀-alkynyl”) carbon atoms, or (“C₂-C₆-alkynyl”) or 2 to 4 (“C₂-C₄-alkynyl”) carbon atoms, and one triple bond in any position. Examples for C₂-C₃-alkynyl are ethynyl, 1-propynyl or 2-propynyl. Examples for C₂-C₄-alkynyl are ethynyl, 1-propynyl, 2-propynyl, 1-butynyl, 2-butynyl, 3-butynyl, 1-methyl-2-propynyl and the like. Examples for C₂-C₆-alkynyl are ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like.

The term “haloalkynyl” as used herein, which may also be expressed as “alkynyl which is substituted by halogen”, refers to unsaturated straight-chain or branched aliphatic non-cyclic hydrocarbon radicals having 2 to 30 (“C₂-C₃₀-haloalkynyl”) or 2 to 20 (“C₂-C₂₀-haloalkynyl”) or 2 to 4 (“C₂-C₄-haloalkynyl”) or 2 to 3 (“C₂-C₃-haloalkynyl”) carbon atoms and a triple bond in any position, where some or all of the hydrogen atoms in these groups are replaced by halogen atoms as mentioned above, in particular fluorine, chlorine and bromine.

The term “cycloalkyl” indicates monocyclic, bicyclic or polycyclic saturated hydrocarbon radicals having in general 3 to 30 (“C₃-C₃₀-cycloalkyl”), or 3 to 20 (“C₃-C₂₀-cycloalkyl”), or 3 to 10 (“C₃-C₁₀-cycloalkyl”), or 3 to 8 (“C₃-C₈-cycloalkyl”) or 3 to 6 (“C₃-C₆-cycloalkyl”) carbon atoms (and of course no heteroatoms) as ring members; i.e. all ring members are carbon atoms. Examples of monocyclic cycloalkyl having 3 to 4 carbon atoms comprise cyclopropyl and cyclobutyl. Examples of monocyclic cycloalkyl having 3 to 5 carbon atoms comprise cyclopropyl, cyclobutyl and cyclopentyl. Examples of monocyclic cycloalkyl having 3 to 6 carbon atoms comprise cyclopropyl, cyclobutyl, cyclopentyl and cyclohexyl. Examples of monocyclic cycloalkyl having 3 to 8 carbon atoms comprise cyclopropyl, cyclobutyl, cyclopentyl, cyclohexyl, cycloheptyl and cyclooctyl. Examples of monocyclic cycloalkyl having 3 to 10 carbon atoms comprise cyclopropyl, cyclobutyl, cyclopentyl, cyclohexyl, cycloheptyl, cyclooctyl, cyclononyl and cyclodecyl.

Bi- or polycyclic saturated hydrocarbon radicals have in general 4 to 30 (“polycyclic C₄-C₃₀-cycloalkyl”), or 4 to 20 (“polycyclic C₄-C₂₀-cycloalkyl”), or 6 to 20 (“polycyclic C₆-C₂₀-cycloalkyl”) carbon atoms (and of course no heteroatoms) as ring members; i.e. all ring members are carbon atoms. The bi- and polycyclic radicals can be condensed, bridged or spiro-bound rings. Examples of bicyclic condensed saturated radicals having 6 to 10 carbon atoms comprise bicyclo[3.1.0]hexyl, bicyclo[3.2.0]heptyl, bicyclo[3.3.0]octyl (1,2,3,3a,4,5,6,6a-octahydropentalenyl), bicyclo[4.2.0]octyl, bicyclo[4.3.0]nonyl (2,3,3a,4,5,6,7,7a-octahydro-1H-indene), bicyclo[4.4.0]decyl (decalinyl) and the like.

Examples of bridged bicyclic condensed saturated radicals having 7 to 10 carbon atoms comprise bicyclo[2.2.1]heptyl, bicyclo[3.1.1]heptyl, bicyclo[2.2.2]octyl, bicyclo[3.2.1]octyl and the like. Examples of bicyclic spiro-bound saturated radicals are spiro[2.2]pentyl, spiro[2.4]heptyl, spiro[4.4]nonyl, spiro[4.5]decyl, spiro[5.5]undecyl and the like. Examples for saturated polycyclic radicals comprise 2,3,4,4a,4b,5,6,7,8,8a,9,9a-dodecahydro-1H-fluorenyl, 1,2,3,4,4a,5,6,7,8,8a,9,9a,10,10a-tetradecahydroanthracenyl, 1,2,3,4,4a,4b,5,6,7,8,8a,9,10,10a-tetradecahydrophenanthrenyl, 2,3,3a,4,5,6,6a,7,8,9,9a,9b-dodecahydro-1H-phenalenyl, adamantly and the like. Preferably, cycloalkyl is monocyclic.

C₃-C₆-Cycloalkyl-C₁-C₁₀-alkyl is a C₁-C₁₀-alkyl group, as defined above, wherein one hydrogen atom is replaced by a C₃-C₆-cycloalkyl group, as defined above. Examples are cyclopropylmethyl, cyclobutylmethyl, cyclopentylmethyl, cyclohexylmethyl, 1-cyclopropylethyl, 2-cyclopropylethyl, 1-cyclobutylethyl, 2-cyclobutylethyl, 1-cyclopentylethyl, 2-cyclopentylethyl, 1-cyclohexylethyl, 2-cyclohexylethyl, 1-cyclopropylpropyl, 2-cyclopropylpropyl, 3-cyclopropylpropyl, 1-cyclobutylpropyl, 2-cyclobutylpropyl, 3-cyclobutylpropyl, 1-cyclopentylpropyl, 2-cyclopentylpropyl, 3-cyclopentylpropyl, 1-cyclohexylpropyl, 2-cyclohexylpropyl, 3-cyclohexylpropyl, 1-cyclohexylbutyl, 2-cyclohexylbutyl, 3-cyclohexylbutyl, 4-cyclohexylbutyl, and the like.

“Alkoxy” is an alkyl group, as defined above, attached via an oxygen atom to the remainder of the molecule; for example a C₁-C₄-alkyl group (“C₁-C₄-aloxy”) attached via an oxygen atom to the remainder of the molecule. “C₁-C₂-Alkoxy” is a C₁-C₂-alkyl group, as defined above, attached via an oxygen atom. “C₁-C₃-Alkoxy” is a C₁-C₃-alkyl group, as defined above, attached via an oxygen atom. C₁-C₂-Alkoxy is methoxy or ethoxy. C₁-C₃-Alkoxy is additionally, for example, n-propoxy and 1-methylethoxy (isopropoxy). C₁-C₄-Alkoxy is additionally, for example, butoxy, 1-methylpropoxy (secbutoxy), 2-methylpropoxy (isobutoxy) or 1,1-dimethylethoxy (tert-butoxy).

The term “haloalkoxy” as used herein denotes in each case a straight-chain or branched alkoxy group, as defined above, having from 1 to 4 carbon atoms (═C₁-C₄-haloalkoxy), wherein the hydrogen atoms of this group are partially or totally replaced with halogen atoms, in particular fluorine atoms (in this case, the radical is also termed fluorinated alkoxy). C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₃-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F₅, 1-(CH₂F)-2-fluoroethoxy, 1-(CH₂Cl)-2-chloroethoxy or 1-(CH₂Br)-2-bromoethoxy. C₁-C₄-Haloalkoxy is additionally, for example, 4-fluorobutoxy, 4-chlorobutoxy, 4-bromobutoxy or nonafluorobutoxy.

Amino is —NH₂.

The term “C₁-C₄-alkylamino” denotes a group C₁-C₄-alkyl-N(H)—. Examples are methylamino, ethylamino, propylamino, isopropylamino, n-butylamino, sec-butylamino, isobutylamino and tert-butylamino.

The term “di-(C₁-C₄-alkyl)-amino” denotes a group (C₁-C₄-alkyl)₂N—. Examples are dimethylamino, diethylamino, ethylmethylamino, dipropylamino, diisopropylamino, methylpropylamino, methylisopropylamino, ethylpropylamino, ethylisopropylamino, n-butyl-methylamino, n-butyl-ethylamino, n-butyl-propylamino, di-n-butylamino, 2-butyl-methylamino, 2-butyl-ethylamino, 2-butyl-propylamino, isobutyl-methylamino, ethyl-isobutylamino, isobutyl-propylamino, tert-butyl-methylamino, tert-butyl-ethylamino, tert-butyl-propylamino and the like.

“Aryl” is a mono-, bi- or polycyclic carbocyclic (i.e. without heteroatoms as ring members) aromatic radical. One example for a monocyclic aromatic radical is phenyl. In bicyclic aryl rings two aromatic rings are condensed, i.e. they share two vicinal C atoms as ring members. One example for a bicyclic aromatic radical is naphthyl. In polycyclic aryl rings, three or more rings are condensed. Examples for polycyclic aryl radicals are phenanthrenyl, anthracenyl, tetracenyl, 1H-benzo[a]phenalenyl, pyrenyl and the like. In the terms of the present invention “aryl” encompasses however also bi- or polycyclic radicals in which not all rings are aromatic, as long as at least one ring is. In R², the attachment point to N has to be on the aromatic moiety. Examples are indanyl, indenyl, tetralinyl, 6,7,8,9-tetrahydro-5H-benzo[7]annulenyl, fluorenyl, 9,10-dihydroanthracenyl, 9,10-dihydrophenanthrenyl, 1H-benzo[a]phenalenyl and the like, and also ring systems in which not all rings are condensed, but for example spiro-bound or bridged, such as benzonorbornyl. In particular, the aryl group has 6 to 40, particularly 6 to 30, more particularly 6 to 22, specifically 6 to 14 or 6 to 10 carbon atoms as ring members.

C₆-C₁₄-Aryl-C₁-C₄-alkyl is a C₁-C₄-alkyl group, as defined above, in which one hydrogen atom is replaced by a C₆-C₁₄-aryl group as defined above (i.e. the attachment to the remainder of the molecule is via the alkyl group). Examples are benzyl, 1-phenylethyl, 2-phenylethyl, 1-phenylpropyl, 2-phenylpropyl, 3-phenylpropyl, 2-phenyl-2-propyl, naphth-1-yl-methyl, naphth-2-yl-methyl, 1-(naphth-1-yl)-ethyl, 1-(naphth-2-yl)-ethyl, 2-(naphth-1-yl)-ethyl, 2-(naphth-2-yl)-ethyl and the like.

Rings termed as heterocyclic rings or heterocyclyl or heteroaromatic rings or heteroaryl or hetaryl contain one or more heteroatoms as ring members, i.e. atoms different from carbon. In the terms of the present invention, these heteroatoms are N, O and S, where S can also be present as a heteroatom group, namely as SO or SO₂. Thus, in the terms of the present invention, rings termed as heterocyclic rings or heterocyclyl or heteroaromatic rings or heteroaryl or hetaryl contain one or more heteroatoms and/or heteroatom groups selected from the group consisting of N, O, S, SO and SO₂ as ring members.

In the terms of the present invention a heterocyclic ring or heterocyclyl is a saturated, partially unsaturated or maximally unsaturated, including aromatic heteromono-, bi- or polycyclic ring (if the ring is aromatic, it is also termed heteroaromatic ring or heteroaryl or hetaryl) containing one or more, in particular 1, 2, 3 or 4 heteroatoms or heteroatom groups independently selected from the group consisting of N, O, S, SO and SO₂ as ring members.

Unsaturated rings contain at least one C—C and/or C—N and/or N—N double bond(s). Maximally unsaturated rings contain as many conjugated C—C and/or C—N and/or N—N double bonds as allowed by the ring size. Maximally unsaturated 5- or 6-membered heteromonocyclic rings are generally aromatic. Exceptions are maximally unsaturated 6-membered rings containing O, S, SO and/or SO₂ as ring members, such as pyran and thiopyran, which are not aromatic. Partially unsaturated rings contain less than the maximum number of C—C and/or C—N and/or N—N double bond(s) allowed by the ring size.

The heterocyclic ring may be attached to the remainder of the molecule via a carbon ring member or via a nitrogen ring member. As a matter of course, the heterocyclic ring contains at least one carbon ring atom. If the ring contains more than one O ring atom, these are not adjacent.

Heterocyclic rings are generally 3- to 30-membered, for example 3- to 20-membered, or 5- to 10-membered or 5- to 6-membered. The heterocyclic rings can be monocyclic, bicyclic or polycyclic.

Heteromonocyclic rings are in particular 3- to 8-membered. Examples of a 3-, 4-, 5-, 6-, 7- or 8-membered saturated heteromonocyclic ring include: Oxiran-2-yl, thiiran-2-yl, aziridin-1-yl, aziridin-2-yl, oxetan-2-yl, oxetan-3-yl, thietan-2-yl, thietan-3-yl, 1-oxothietan-2-yl, 1-oxothietan-3-yl, 1,1-dioxothietan-2-yl, 1,1-dioxothietan-3-yl, azetidin-1-yl, azetidin-2-yl, azetidin-3-yl, tetrahydrofuran-2-yl, tetrahydrofuran-3-yl, tetrahydrothien-2-yl, tetrahydrothien-3-yl, 1-oxotetrahydrothien-2-yl, 1,1-dioxotetrahydrothien-2-yl, 1-oxotetrahydrothien-3-yl, 1,1-dioxotetrahydrothien-3-yl, pyrrolidin-1-yl, pyrrolidin-2-yl, pyrrolidin-3-yl, pyrazolidin-1-yl, pyrazolidin-3-yl, pyrazolidin-4-yl, pyrazolidin-5-yl, imidazolidin-1-yl, imidazolidin-2-yl, imidazolidin-4-yl, oxazolidin-2-yl, oxazolidin-3-yl, oxazolidin-4-yl, oxazolidin-5-yl, isoxazolidin-2-yl, isoxazolidin-3-yl, isoxazolidin-4-yl, isoxazolidin-5-yl, thiazolidin-2-yl, thiazolidin-3-yl, thiazolidin-4-yl, thiazolidin-5-yl, isothiazolidin-2-yl, isothiazolidin-3-yl, isothiazolidin-4-yl, isothiazolidin-5-yl, 1,2,4-oxadiazolidin-2-yl, 1,2,4-oxadiazolidin-3-yl, 1,2,4-oxadiazolidin-4-yl, 1,2,4-oxadiazolidin-5-yl, 1,2,4-thiadiazolidin-2-yl, 1,2,4-thiadiazolidin-3-yl, 1,2,4-thiadiazolidin-4-yl, 1,2,4-thiadiazolidin-5-yl, 1,2,4-triazolidin-1-yl, 1,2,4-triazolidin-3-yl, 1,2,4-triazolidin-4-yl, 1,3,4-oxadiazolidin-2-yl, 1,3,4-oxadiazolidin-3-yl, 1,3,4-thiadiazolidin-2-yl, 1,3,4-thiadiazolidin-3-yl, 1,3,4-triazolidin-1-yl, 1,3,4-triazolidin-2-yl, 1,3,4-triazolidin-3-yl, tetrahydropyran-2-yl, tetrahydropyran-3-yl, tetrahydropyran-4-yl, 1,3-dioxan-2-yl, 1,3-dioxan-4-yl, 1,3-dioxan-5-yl, 1,4-dioxan-2-yl, piperidin-1-yl, piperidin-2-yl, piperidin-3-yl, piperidin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, 1,3,5-hexahydrotriazin-1-yl, 1,3,5-hexahydrotriazin-2-yl, 1,2,4-hexahydrotriazin-1-yl, 1,2,4-hexahydrotriazin-2-yl, 1,2,4-hexahydrotriazin-3-yl, 1,2,4-hexahydrotriazin-4-yl, 1,2,4-hexahydrotriazin-5-yl, 1,2,4-hexahydrotriazin-6-yl, morpholin-2-yl, morpholin-3-yl, morpholin-4-yl, thiomorpholin-2-yl, thiomorpholin-3-yl, thiomorpholin-4-yl, 1-oxothiomorpholin-2-yl, 1-oxothiomorpholin-3-yl, 1-oxothiomorpholin-4-yl, 1,1-dioxothiomorpholin-2-yl, 1,1-dioxothiomorpholin-3-yl, 1,1-dioxothiomorpholin-4-yl, azepan-1-, -2-, -3- or -4-yl, oxepan-2-, -3-, -4- or -5-yl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl, oxocane, thiocane, azocanyl, [1,3]diazocanyl, [1,4]diazocanyl, [1,5]diazocanyl, [1,5]oxazocanyl and the like.

Examples of a 3-, 4-, 5-, 6-, 7- or 8-membered partially unsaturated heteromonocyclic ring include: 2,3-dihydrofuran-2-yl, 2,3-dihydrofuran-3-yl, 2,4-dihydrofuran-2-yl, 2,4-dihydrofuran-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl, tetrahydro-1,4-dioxepinyl, 1,2,3,4,5,6-hexahydroazocine, 2,3,4,5,6,7-hexahydroazocine, 1,2,3,4,5,8-hexahydroazocine, 1,2,3,4,7,8-hexahydroazocine, 1,2,3,4,5,6-hexahydro-[1,5]diazocine,1,2,3,4,7,8-hexahydro-[1,5]diazocine and the like.

Examples of a 3-, 4-, 5-, 6-, 7- or 8-membered maximally unsaturated (but not aromatic) heteromonocyclic ring are pyran-2-yl, pyran-3-yl, pyran-4-yl, thiopryran-2-yl, thiopryran-3-yl, thiopryran-4-yl, 1-oxothiopryran-2-yl, 1-oxothiopryran-3-yl, 1-oxothiopryran-4-yl, 1,1-dioxothiopryran-2-yl, 1,1-dioxothiopryran-3-yl, 1,1-dioxothiopryran-4-yl, 2H-oxazin-2-yl, 2H-oxazin-3-yl, 2H-oxazin-4-yl, 2H-oxazin-5-yl, 2H-oxazin-6-yl, 4H-oxazin-3-yl, 4H-oxazin-4-yl, 4H-oxazin-5-yl, 4H-oxazin-6-yl, 6H-oxazin-3-yl, 6H-oxazin-4-yl, 7H-oxazin-5-yl, 8H-oxazin-6-yl, 2H-1,3-oxazin-2-yl, 2H-1,3-oxazin-4-yl, 2H-1,3-oxazin-5-yl, 2H-1,3-oxazin-6-yl, 4H-1,3-oxazin-2-yl, 4H-1,3-oxazin-4-yl, 4H-1,3-oxazin-5-yl, 4H-1,3-oxazin-6-yl, 6H-1,3-oxazin-2-yl, 6H-1,3-oxazin-4-yl, 6H-1,3-oxazin-5-yl, 6H-1,3-oxazin-6-yl, 2H-1,4-oxazin-2-yl, 2H-1,4-oxazin-3-yl, 2H-1,4-oxazin-5-yl, 2H-1,4-oxazin-6-yl, 4H-1,4-oxazin-2-yl, 4H-1,4-oxazin-3-yl, 4H-1,4-oxazin-4-yl, 4H-1,4-oxazin-5-yl, 4H-1,4-oxazin-6-yl, 6H-1,4-oxazin-2-yl, 6H-1,4-oxazin-3-yl, 6H-1,4-oxazin-5-yl, 6H-1,4-oxazin-6-yl, 1,4-dioxine-2-yl, 1,4-oxathiin-2-yl, 1H-azepine, 1H-[1,3]-diazepine, 1H-[1,4]-diazepine, [1,3]diazocine, [1,5]diazocine, [1,5]diazocine and the like.

Heteroaromatic monocyclic rings are in particular 5- or 6-membered. Examples for 5- or 6-membered monocyclic heteroaromatic rings are 2-furyl, 3-furyl, 2-thienyl, 3-thienyl, 1-pyrrolyl, 2-pyrrolyl, 3-pyrrolyl, 1-pyrazolyl, 3-pyrazolyl, 4-pyrazolyl, 5-pyrazolyl, 1-imidazolyl, 2-imidazolyl, 4-imidazolyl, 5-imidazolyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 3-isothiazolyl, 4-isothiazolyl, 5-isothiazolyl, 1,3,4-triazol-1-yl, 1,3,4-triazol-2-yl, 1,3,4-triazol-3-yl, 1,2,3-triazol-1-yl, 1,2,3-triazol-2-yl, 1,2,3-triazol-4-yl, 1,2,5-oxadiazol-3-yl, 1,2,3-oxadiazol-4-yl, 1,2,3-oxadiazol-5-yl, 1,3,4-oxadiazol-2-yl, 1,2,5-thiadiazol-3-yl, 1,2,3-thiadiazol-4-yl, 1,2,3-thiadiazol-5-yl, 1,3,4-thiadiazol-2-yl, 2-pyridinyl, 3-pyridinyl, 4-pyridinyl, 1-oxopyridin-2-yl, 1-oxopyridin-3-yl, 1-oxopyridin-4-yl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl, 1,2,4-triazin-5-yl, 1,2,3,4-tetrazin-1-yl, 1,2,3,4-tetrazin-2-yl, 1,2,3,4-tetrazin-5-yl and the like.

“Heterobicyclic rings” or “heterobicyclyl” contain two rings which have at least one ring atom in common. At least one of the two rings contains a heteroatom or heteroatom group selected from the group consisting of N, O, S, SO and SO₂ as ring member. The term comprises condensed (fused) ring systems, in which the two rings have two neighboring ring atoms in common, as well as spiro systems, in which the rings have only one ring atom in common, and bridged systems with at least three ring atoms in common. In terms of the present invention, the heterobicyclic rings include throughout aromatic bicyclic ring systems; these are also termed heteroaromatic bicyclic rings or bicycyclic het(ero)aryl or heterobiaryl. The heterobicyclic rings are preferably 7-, 8-, 9-, 10- or 11-membered. The heteroaromatic bicyclic rings are preferably 9-, 10- or 11-membered. Throughout heteroaromatic heterobicyclic rings are 9- or 10-membered.

### Examples for Fused Systems:

Examples for a 7-, 8-, 9-, 10- or 11-membered saturated heterobicyclic ring containing 1, 2 or 3 (or 4) heteroatoms or heteroatom groups selected from the group consisting of N, O, S, NO, SO and SO₂, as ring members are:

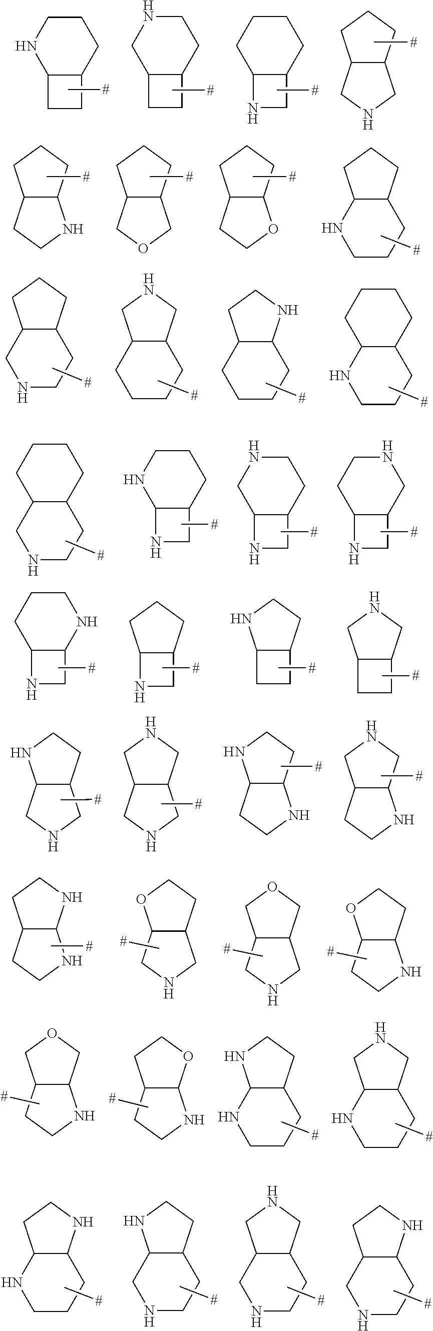

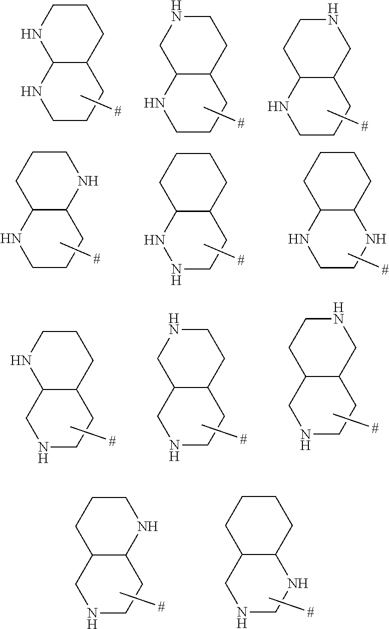

Examples for a 7-, 8-, 9-, 10- or 11-membered partially unsaturated heterobicyclic ring containing 1, 2 or 3 (or 4) heteroatoms or heteroatom groups selected from the group consisting of N, O, S, SO and SO₂, as ring members are:

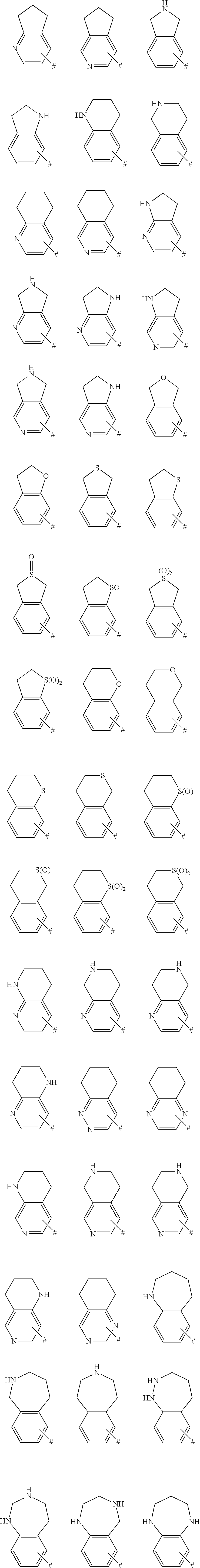

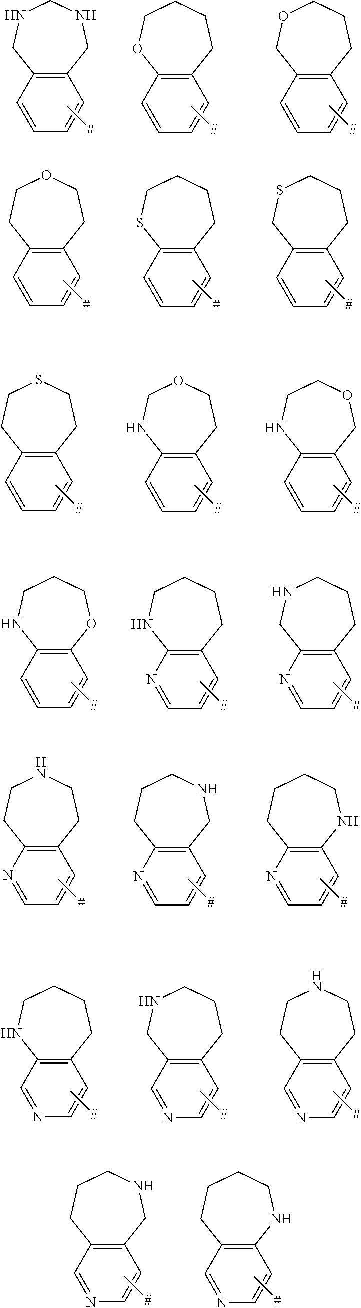

Examples for a 7-, 8-, 9-, 10- or 11-membered maximally unsaturated (but not throughout heteroaromatic) heterobicyclic ring containing 1, 2 or 3 (or 4) heteroatoms or heteroatom groups selected from the group consisting of N, O, S, SO and SO₂, as ring members are:

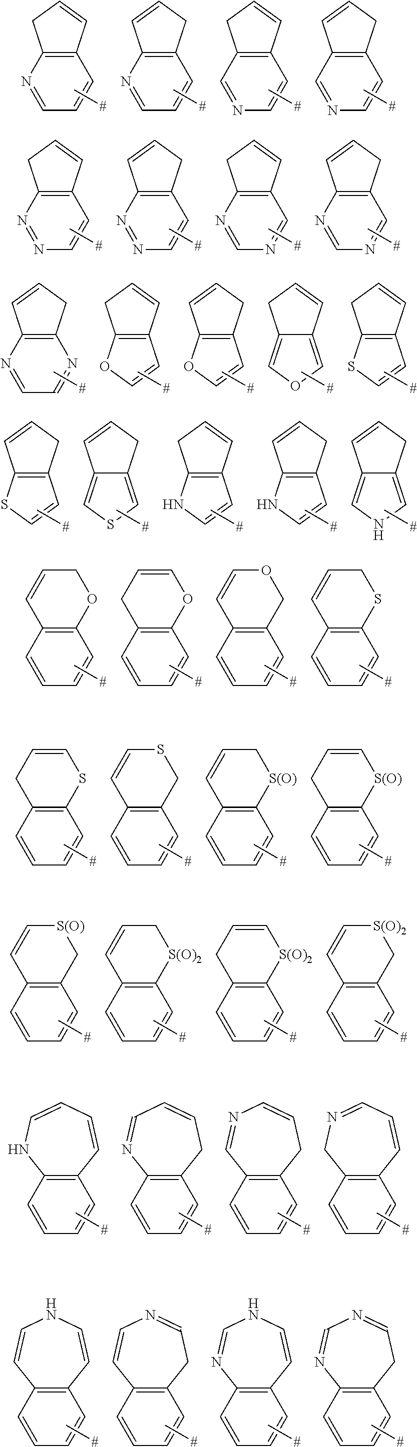

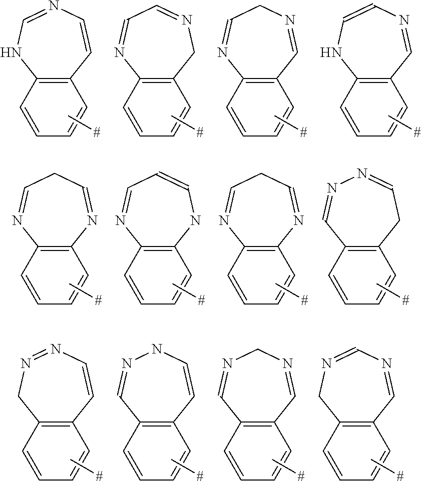

Examples for a 9- or 10-membered maximally unsaturated, throughout heteroaromatic heterobicyclic ring containing 1, 2 or 3 (or 4) heteroatoms or heteroatom groups selected from the group consisting of N, O, S, SO and SO₂, as ring members are:

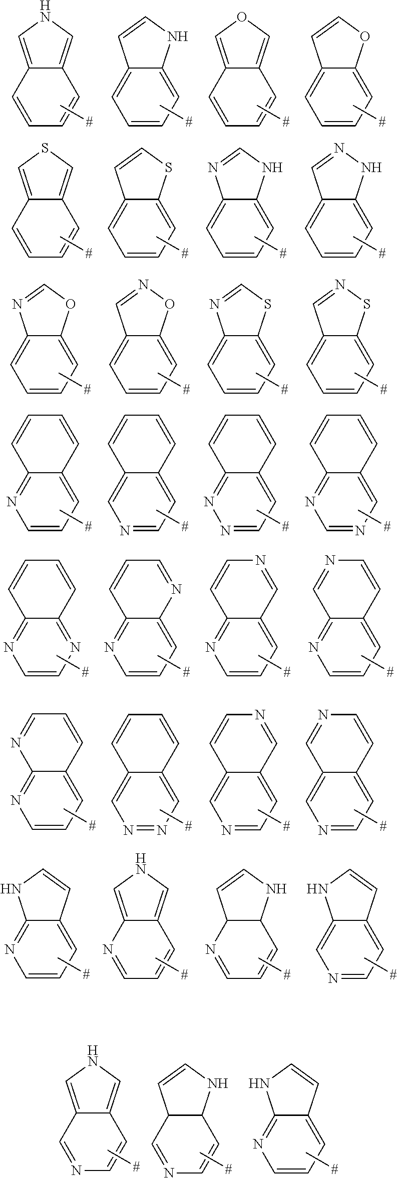

Examples for spiro-bound 7-, 8-, 9-, 10- or 11-membered heterobicyclic rings containing 1, 2 or 3 (or 4) heteroatoms or heteroatom groups selected from the group consisting of N, O, S, NO, SO and SO₂, as ring members are

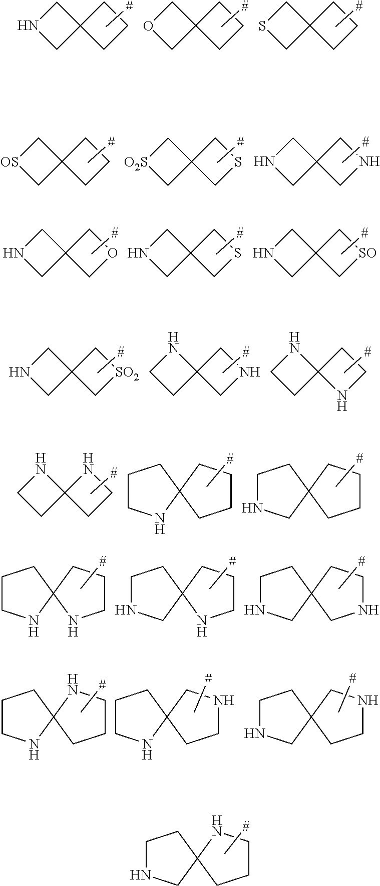

Examples for bridged 7-, 8-, 9-, 10- or 11-membered heterobicyclic rings containing 1, 2 or 3 (or 4) heteroatoms or heteroatom groups selected from the group consisting of N, O, S, NO, SO and SO₂, as ring members are

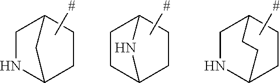

and the like.

In the above structures # denotes the attachment point to the remainder of the molecule. The attachment point is not restricted to the ring on which this is shown, but can be on either of the two rings, and may be on a carbon or on a nitrogen ring atom. If the rings carry one or more substituents, these may be bound to carbon and/or to nitrogen ring atoms.

Polycyclic heterocyclic rings (polyheterocyclyl) contain three or more rings, each of which having at least one ring atom in common with at least one of the other rings of the polycyclic system. The rings can be condensed, spiro-bound or bridged; mixed systems (e.g. one ring is spiro-bound to a condensed system, or a bridged system is condensed with another ring) are also possible. Throughout aromatic rings are not encompassed in the polycyclic heterocyclic ring (polyheterocyclyl); these are termed polycyclic heteroaromatic rings or heteropolyaryls.

If the heterocyclic/heteroaromatic rings are substituted, the substituents can be bound both to carbon ring atoms and to secondary nitrogen ring atoms.

If R³ forms a saturated or unsaturated 2-, 3- or 4-membered linking group to a carbon or nitrogen ring atom of the aryl or heteroaromatic ring R²; where the linking group may comprise 1 or 2 heteroatoms or heteroatom groups selected from, N, O, S, SO and SO₂, this results in a bicyclic heterocyclic ring system if R² is monocyclic or in a polycyclic heterocyclic ring system if R² is bicyclic or polycyclic, where the resulting ring system contains the nitrogen atom of NR²R³ as ring member and is bound via this nitrogen ring member to CO. If the linking group R³ is bound to the ring atom of R² neighbouring the ring atom which forms the attachment point to N (of the group NR²R³), the resulting ring is condensed to the (hetero)aromatic ring R². If the linking group R³ is bound to another ring atom of R², the resulting ring system is a bridged one. To name just a few illustrative examples for thusly resulting ring systems NR²R³, mention be made of 2,3-dihydroindol-1-yl (R² is phenyl, R³ forms —CH₂CH₂— attached ortho to the attachment point of phenyl to N), 1,2,3,4-tetrahydroquinolin-1-yl (R² is phenyl, R³ forms —CH₂CH₂CH₂— attached ortho to the attachment point of phenyl to N), indol-1-yl (R² is phenyl, R³ forms —CH═CH— attached ortho to the attachment point of phenyl to N), 1,2-dihydroquinolin-1-yl (R² is phenyl, R³ forms —CH₂CH═CH— attached ortho to the attachment point of phenyl to N), 1,2,3,4-tetrahydro-1,2-napthyridin-1-yl (R² is phenyl, R³ forms —NHCH₂CH₂— attached ortho to the attachment point of phenyl to N), 1,2,3,4-tetrahydro-1,3-napthyridin-1-yl (R² is phenyl, R³ forms —CH₂NHCH₂— attached ortho to the attachment point of phenyl to N), 1,2,3,4-tetrahydro-1,4-napthyridin-1-yl (R² is phenyl, R³ forms —CH₂CH₂NH— attached ortho to the attachment point of phenyl to N), 1,2,3,4-tetrahydro-1,5-napthyridin-1-yl (R² is pyridin-3-yl, R³ forms —CH₂CH₂CH₂— attached in 2-position of the pyridyl ring), 1,2,3,4-tetrahydro-1,8-napthyridin-1-yl (R² is pyridine-2-yl, R³ forms —CH₂CH₂CH₂— attached in 3-position of the pyridyl ring), and the like.

A is a divalent aliphatic, cycloaliphatic, aromatic, aromatic-aliphatic or heterocyclic moiety.

Divalent aliphatic radicals are those which contain no cycloaliphatic, carboaromatic or heterocyclic constituents. Examples are alkylene (alkanediyl), alkenylene (alkenediyl), and alkynylene (alkynediyl) radicals.

Divalent cycloaliphatic radicals contain one or more, e.g., one or two, cycloaliphatic moieties; however, they contain no carboaromatic or heterocyclic constituents. The cycloaliphatic radicals may be substituted by aliphatic radicals, but bonding sites for the CONR²R³ or COOR⁴ groups are located on the cycloaliphatic radical.

Divalent aliphatic-cycloaliphatic radicals contain not only at least one divalent aliphatic radical, but also at least one divalent cycloaliphatic radical, one of the two bonding sites for the two CONR²R³ groups or for the two COOR⁴ groups being located on an aliphatic radical and the other on a cycloaliphatic radical.

Divalent aromatic radicals contain one or more, e.g., one or two, carboaromatic radicals; however, they contain no cycloaliphatic or heterocyclic constituents. The aromatic radicals may be substituted by aliphatic radicals, but both bonding sites for the two CONR²R³ groups or for the two COOR⁴ groups are located on the aromatic radical(s). Divalent aromatic-aliphatic (short: araliphatic) radicals are divalent radicals containing at least one aromatic and at least one aliphatic moiety. To be more precise, they contain at least one divalent aliphatic radical and at least one divalent carboaromatic radical; one of the two bonding sites for the two CONR²R³ groups or for the two COOR⁴ groups being located on an aliphatic radical and the other on an aromatic radical. Divalent heterocyclic radicals contain one or more, e.g., one or two, heterocyclic radicals; however, they contain no cycloaliphatic or purely carboaromatic constituents. The heterocyclic radicals may be substituted by aliphatic radicals, but both bonding sites for the two CONR²R³ groups or for the two COOR⁴ groups are located on the heterocyclic radical(s).

Carboaromatic means that the aromatic system is composed of carbon atoms only, such as in phenyl or naphthyl. Heterocyclic encompasses heteroaromatic.

Alkylene is a linear or branched divalent alkanediyl radical. C₁-C₄-Alkylene (=C₁-C₄-alkanediyl) is a linear or branched divalent alkyl radical having 1 to 4 carbon atoms. Examples are —CH₂—, —CH₂CH₂—, —CH(CH₃)—, —CH₂CH₂CH₂—, —CH(CH₃)CH₂—, —CH₂CH(CH₃)—, —C(CH₃)₂—, —CH₂CH₂CH₂CH₂—, —CH(CH₃)CH₂CH₂—, —CH₂CH₂CH(CH₃)—, —C(CH₃)₂CH₂—, —CH₂C(CH₃)₂—. C₁-C₈-Alkylene (═C₁-C₈-alkanediyl) is a linear or branched divalent alkyl radical having 1 to 8 carbon atoms. Examples are —CH₂—, —CH₂CH₂—, —CH₂CH₂CH₂—, —CH(CH₃)CH₂—, —CH₂CH(CH₃)—, —C(CH₃)₂—, —CH₂CH₂CH₂CH₂—, —CH(CH₃)CH₂CH₂—, —CH₂CH₂CH(CH₃)—, —C(CH₃)₂CH₂—, —CH₂C(CH₃)₂—, —(CH₂)₅—, —(CH₂)₆—, —(CH₂)₇—, —(CH₂)₈—, and positional isomers thereof. Linear C₁-C₈-alkylene is —CH₂—, —CH₂CH₂—, —CH₂CH₂CH₂—, —CH₂CH₂CH₂CH₂—, —(CH₂)₅—, —(CH₂)₆—, —(CH₂)₇— or —(CH₂)₈—.

Alkenylene is a linear or branched divalent alkenediyl radical with one or more double bonds in any position (provided they are not cumulated). C₂-C₈-Alkylene (═C₂-C₈-alkenediyl) is a linear or branched divalent alkyl radical having 2 to 8 carbon atoms. Examples are —CH═CH—, —C(═CH₂)—, —CH₂—CH═CH—, —CH═CH—CH₂—, —CH₂—C(═CH₂)—, —C(═CH₂)—CH₂—, CH₂—CH═CH—CH₂— and the like.

Cycloalkylene is a divalent cycloalkanediyl. C₃-C₆-Cycloalkanediyl is a divalent cycloalkanediyl having 3 to 6 carbon atoms. Examples are cyclopropane-1,1-diyl, cyclopropane-1,2-diyl, cyclobutane-1,1-diyl, cyclobutane-1,2-diyl, cyclobutane-1,3-diyl, cyclopentene-1,1-diyl, cyclopentene-1,2-diyl, cyclopentene-1,3-diyl, cyclohexane-1,1-diyl, cyclohexane-1,2-diyl, cyclohexane-1,3-diyl, cyclohexane-1,4-diyl.

Examples for (carbo)aromatic divalent radicals are the phenylenes, the naphthylenes and the like. Phenylene is 1,2-phenylene (benzene-1,2-diyl), 1,3-phenylene (benzene-1,3-diyl) or 1,4-phenylene (benzene-1,4-diyl).

Amine (III) is aromatic. This means that the nitrogen atom of NR²R³ is directly linked to an aromatic or heteroaromatic ring. This includes also (bi- or polycyclic) ring systems which are only partly aromatic because one of the rings of this ring systems is not aromatic (as is the case, for example, if NR²R³ forms the above-mentioned 2,3-dihydroinol-1-yl, 1,2,3,4-tetrahydroquinolin-1-yl, 1,2-dihydroquinolin-1-yl, 1,2,3,4-tetrahydro-1,2-, -1,3-, -1,4-, -1,5- or -1,8-napthyridin-1-yl); provided that N (of the group NR²R³) is directly bound to an aromatic or heteroaromatic ring.

An alkoxide or alcoholate is an anion R—O⁻, where R is an alkyl group. Examples for C₁-C₄-alkoxides are methanolate (=methoxide; R═CH₃), ethanolate (=ethoxide; R=ethyl), propanolate (=propoxide; R=n-propyl)), isopropanolate (=isopropoxide; R=isopropyl), n-butanolate (=n-butoxide; R=n-butyl), sec-butanolate (=sec-butoxide; R=sec-butyl), isobutanolate (=isobutoxide; R=isobutyl) or tert-butanolate (=tert-butoxide; R=tert-butyl). Examples for C₁-C₁₀-alkoxides are, in addition to those mentioned for C₁-C₄-alkoxides, n-pentanolate, n-hexanolate, n-heptanolate, n-octanolate, 2-ethylhexanolate, n-nonanolate, n-decanolate, 2-propyleheptanolate and other positional isomers thereof.

Lewis acids are electron pair acceptors. Generally, they include compounds in which an atom has no noble gas configuration, for example main group atoms with incomplete or unstable electron octet, such as boron or aluminium in B(CH₃)₃, B(OH)₃, BF₃ or AlCl₃. In addition to the boron and aluminum compounds mentioned above, examples are metal salts which are not in form of complexes in which the central metal has a stable noble gas configuration (a noble gas configuration is often obtained due to ligands, such as water or the counteranion if this is bi- or polydentate; without such ligands, or if such ligands are easily displaced (see below remarks to Lewis acid precursors), metal salts are generally Lewis acids), or metal complexes of metal (ions) which do not have a noble gas configuration, e.g. transition metal complexes of metals with incompletely filled d-orbitals, e.g. Cr³⁺ complexes. The term “Lewis acid”, as used herein, also encompasses Lewis acid precursors, as long as these are converted into the proper Lewis acid under the reaction conditions for the present amidation reaction. Examples are metal carbonyl complexes, such as Cr(CO)₆, MnBr(CO)₅ or Mn₂(CO)₁₀, which, under reaction conditions typical for amidation reactions, especially under elevated temperature, lose a CO ligand and/or decompose (for instance, the binuclear complex Mn₂(CO)₁₀ may dissociate to the fragment Mn(CO)₅), forming thus a coordinatively unsaturated complex with Lewis acidity). Lewis acids used in the present amidations are preferably metal salts or metal complexes in which the metal has no noble gas configuration, and Lewis acid precursors which, under the reaction conditions for the present amidation reaction, are converted into the proper Lewis acids and which are in the form of metal complexes, especially metal carbonyl complexes.

### Embodiments (E.x) of the Invention

General and preferred embodiments E.x are summarized in the following, non-exhaustive list. Further preferred embodiments become apparent from the paragraphs following this list.
E.1. A method for preparing an amide of the formula (I-1) or a diamide of the formula (I-2)

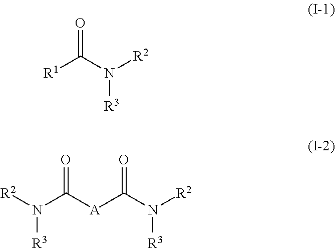

where
R¹ is selected from the group consisting of hydrogen, C₁-C₃₀-alkyl which is unsubstituted or carries m radicals R^{a}, C₁-C₃₀-haloalkyl which is unsubstituted or carries m radicals R^{a}, C₂-C₃₀-alkenyl which is unsubstituted or carries m radicals R^{a}, C₂-C₃₀-haloalkenyl which is unsubstituted or carries m radicals R^{a}, C₂-C₃₀-alkynyl which is unsubstituted or carries m radicals R^{a}, C₂-C₃₀-haloalkynyl which is unsubstituted or carries m radicals R^{a}, C₃-C₃₀-cycloalkyl which is unsubstituted or carries m radicals R^{b}, C₆-C₂₂-aryl which is unsubstituted or carries m radicals R^{b}, and a 3- to 30-membered saturated, partially unsaturated or maximally unsaturated heterocyclic ring containing 1, 2, 3 or 4 heteroatoms or heteroatom groups selected from, N, O, S, SO and SO₂ as ring members, which is unsubstituted or carries m radicals R^{b};
R² is C₆-C₂₂-aryl which is unsubstituted or carries m radicals R^{b}, or is a 5- to 30-membered heteroaromatic ring containing 1, 2, 3 or 4 heteroatoms selected from N, O and S as ring members, where the heteroaromatic ring is unsubstituted or carries m radicals R^{b};
R³ is selected from the group consisting of hydrogen, C₁-C₃₀-alkyl which is unsubstituted or carries m radicals R^{a}, C₁-C₃₀-haloalkyl which is unsubstituted or carries m radicals R^{a}, C₂-C₃₀-alkenyl which is unsubstituted or carries m radicals R^{a}, C₂-C₃₀-haloalkenyl which is unsubstituted or carries m radicals R^{a}, C₂-C₃₀-alkynyl which is unsubstituted or carries m radicals R^{a}, C₂-C₃₀-haloalkynyl which is unsubstituted or carries m radicals R^{a}, C₃-C₃₀-cycloalkyl which is unsubstituted or carries m radicals R^{b}, C₆-C₂₂-aryl which is unsubstituted or carries m radicals R^{b}, and a 3- to 30-membered saturated, partially unsaturated or maximally unsaturated heterocyclic ring containing 1, 2, 3 or 4 heteroatoms or heteroatom groups selected from, N, O, S, SO and SO₂ as ring members, which is unsubstituted or carries m radicals R^{b};
or
R³ forms a saturated or unsaturated 2-, 3- or 4-membered linking group to a carbon or nitrogen ring atom of the aryl or heteroaromatic ring R²; where the linking group may comprise 1 or 2 heteroatoms or heteroatom groups selected from, N, O, S, SO and SO₂; where the linking group may carry 1, 2 or 3 radicals selected from the group consisting of halogen, cyano, C₁-C₄-alkyl, C₁-C₄-haloalkyl, C₁-C₄-alkoxy and C₁-C₄-haloalkoxy;
A is a divalent aliphatic, cycloaliphatic, aliphatic-cycloaliphatic aromatic, aromatic-aliphatic or heterocyclic moiety;
each R^{a} is independently selected from the group consisting of cyano, nitro, hydroxyl, C₁-C₄-alkoxy, C₁-C₄-haloalkoxy, C(═O)R^{c}, amino, C₁-C₄-alkylamino, di-(C₁-C₄-alkyl)-amino, C₃-C₂₀-cycloalkyl, C₆-C₂₂-aryl which is unsubstituted or carries m radicals R^{d}, and a 3- to 20-membered saturated, partially unsaturated or maximally unsaturated heterocyclic ring containing 1, 2, 3 or 4 heteroatoms or heteroatom groups selected from, N, O, S, SO and SO₂ as ring members, which is unsubstituted or carries m radicals R^{d};
each R^{b} is independently selected from the group consisting of halogen, cyano, nitro, hydroxyl, C₁-C₄-alkoxy, C₁-C₄-haloalkoxy, amino, C₁-C₄-alkylamino, di-(C₁-C₄-alkyl)-amino, NR^{e}R^{f}, C(═O)NR^{e}R^{f}, C₁-C₂₀-alkyl, C₁-C₂₀-haloalkyl, C₂-C₂₀-alkenyl, C₂-C₂₀-haloalkenyl, C₂-C₂₀-alkynyl, C₂-C₂₀-haloalkynyl, C₃-C₂₀-cycloalkyl, C₆-C₂₂-aryl which is unsubstituted or carries m radicals R^{d}, and a 3- to 20-membered saturated, partially unsaturated or maximally unsaturated heterocyclic ring containing 1, 2, 3 or 4 heteroatoms or heteroatom groups selected from, N, O, S, SO and SO₂ as ring members, which is unsubstituted or carries m radicals R^{d};
each R^{c} is independently selected from the group consisting of C₁-C₄-alkyl, C₁-C₄-haloalkyl, C₁-C₄-alkyl which carries a group NR^{e}R^{f}, C₁-C₄-alkoxy and C₁-C₄-haloalkoxy;
each R^{d} is independently selected from the group consisting of halogen, cyano, hydroxyl, C₁-C₄-alkyl, C₁-C₄-haloalkyl, C₁-C₄-alkoxy and C₁-C₄-haloalkoxy;
each R^{e} is independently selected from the group consisting of hydrogen and C₁-C₄-alkyl;
each R^{f} is independently selected from the group consisting of —C(═O)-phenyl and phenyl which is unsubstituted or substituted by 1, 2 or 3 radicals selected from the group consisting of halogen, cyano, C₁-C₄-alkyl, C₁-C₄-haloalkyl, C₁-C₄-alkoxy and C₁-C₄-haloalkoxy;
each m is independently 1, 2, 3, 4 or 5;
which method comprises reacting an ester compound (II) of the formula (II-1) or (II-2)

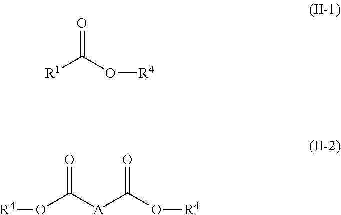

wherein
R¹ and A are as defined above; and
R⁴ is selected from the group consisting of C₁-C₃₀-alkyl, C₆-C₁₄-aryl and C₆-C₁₄-aryl-C₁-C₄-alkyl;
with an amine of the formula (III)

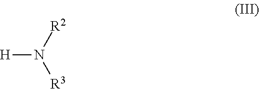

wherein R² and R³ are as defined above,
in the presence of an alkali metal-containing base and a Lewis acid;
where the reaction is carried out under anhydrous conditions, where the water content in the reaction mixture is at most 0.15% by weight, relative to the total weight of the reaction mixture.
E.2. The method according to embodiment E.1, where R¹ is selected from the group consisting of C₁-C₂₀-alkyl, C₁-C₄-alkyl which carries 1 or 2 radicals R^{a}, C₂-C₂₀-alkenyl, C₂-C₄-alkenyl which carries a phenyl ring, C₃-C₆-cycloalkyl which is unsubstituted or carries m radicals R^{b}, C₆-C₁₀-aryl which is unsubstituted or carries m radicals R^{b}, and a 5- to 10-membered heteroaromatic ring containing 1, 2, 3 or 4 heteroatoms selected from, N, O and S as ring members, which is unsubstituted or carries m radicals R^{b};
   where
   each R^{a} is independently C₁-C₄-alkoxy, C₁-C₄-haloalkoxy, C(═O)R^{c} or phenyl; and
   each R^{b} is independently halogen, cyano, C₁-C₄-alkyl, C₁-C₄-haloalkyl, C₁-C₄-alkoxy, C₁-C₄-haloalkoxy and a 5- or 6-membered heteroaromatic ring containing 1, 2, 3 or 4 heteroatoms selected from, N, O and S as ring members, which is unsubstituted or carries m radicals R^{d}; where each R^{d} is independently selected from the group consisting of halogen, cyano, C₁-C₄-alkyl, C₁-C₄-haloalkyl, C₁-C₄-alkoxy and C₁-C₄-haloalkoxy.
E.3. The method according to embodiment E.2, where R¹ is selected from the group consisting of C₁-C₂₀-alkyl, C₁-C₄-alkyl which carries a phenyl ring, C₂-C₂₀-alkenyl, C₂-C₄-alkenyl which carries a phenyl ring, C₆-C₁₀-aryl which is unsubstituted or carries m radicals R^{b}, and a 5- to 10-membered heteroaromatic ring containing 1, 2, 3 or 4 heteroatoms selected from, N, O and S as ring members, which is unsubstituted or carries m radicals R^{b};
   where each R^{b} is independently halogen, cyano, C₁-C₄-alkyl, C₁-C₄-haloalkyl, C₁-C₄-alkoxy and C₁-C₄-haloalkoxy.
E.4. The method according to embodiment E.3, where R¹ is selected from the group consisting of C₁-C₄-alkyl which carries a phenyl ring, C₂-C₂₀-alkenyl, C₂-C₄-alkenyl which carries a phenyl ring, phenyl which is unsubstituted or carries m radicals R^{b}, and a 5- or 6-membered heteroaromatic ring containing 1, 2 or 3 heteroatoms selected from, N, O and S as ring members, which is unsubstituted or carries m radicals R^{b};
   where each R^{b} is independently halogen, cyano, C₁-C₄-alkyl, C₁-C₄-haloalkyl, C₁-C₄-alkoxy and C₁-C₄-haloalkoxy; and m is 1, 2 or 3.
E.5. The method according to embodiment E.4, where R¹ is selected from the group consisting of C₁-C₂-alkyl which carries a phenyl ring, C₂-C₂₀-alkenyl, C₂-alkenyl which carries a phenyl ring, phenyl which is unsubstituted or carries m radicals R^{b}, and a 5- or 6-membered heteroaromatic ring containing 1 or 2 nitrogen atoms as ring members which is unsubstituted or carries m radicals R^{b};
   where each R^{b} is independently halogen, cyano, C₁-C₄-alkyl, C₁-C₄-haloalkyl, C₁-C₄-alkoxy and C₁-C₄-haloalkoxy; and m is 1, 2 or 3.
E.6. The method according to embodiment E.4, where R¹ is phenyl which is unsubstituted or carries m radicals R^{b}, or a 5- or 6-membered heteroaromatic ring containing 1, 2 or 3 heteroatoms selected from, N, O and S as ring members, which is unsubstituted or carries m radicals R^{b};
   where each R^{b} is independently halogen, cyano, C₁-C₄-alkyl, C₁-C₄-haloalkyl, C₁-C₄-alkoxy and C₁-C₄-haloalkoxy; and m is 1, 2 or 3.
E.7. The method according to embodiment E.6, where R¹ is phenyl which is unsubstituted or carries m radicals R^{b}, or a 5- or 6-membered heteroaromatic ring containing 1 or 2 nitrogen atoms as ring members, which is unsubstituted or carries m radicals R^{b};
   where each R^{b} is independently halogen, cyano, C₁-C₄-alkyl, C₁-C₄-haloalkyl, C₁-C₄-alkoxy and C₁-C₄-haloalkoxy; and m is 1, 2 or 3.
E.8. The method according to any of embodiments E.5 or E.7, where R¹ is phenyl which is unsubstituted or carries m radicals R^{b}, or pyridyl which is unsubstituted or carries m radicals R^{b}; where each R^{b} is independently halogen, cyano, C₁-C₄-alkyl and C₁-C₄-alkoxy; and m is 1, 2 or 3.
E.9. The method according to any of the preceding embodiments, where R² is selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b};
   where
   each R^{b} is independently selected from the group consisting of halogen, cyano, hydroxyl, nitro, C(═O)NR^{e}R^{f}, C₁-C₄-alkyl, C₁-C₄-haloalkyl, C₁-C₄-alkoxy, C₁-C₄-haloalkoxy and phenyl which is unsubstituted or carries m radicals R^{d};
   each R^{d} is independently selected from the group consisting of halogen, C₁-C₄-alkyl and C₁-C₄-haloalkyl;
   each R^{e} is independently selected from the group consisting of hydrogen and C₁-C₄-alkyl;
   each R^{f} is independently selected from the group consisting of —C(═O)-phenyl and phenyl which is unsubstituted or substituted by 1, 2 or 3 radicals selected from the group consisting of halogen, cyano, C₁-C₄-alkyl, C₁-C₄-haloalkyl, C₁-C₄-alkoxy and C₁-C₄-haloalkoxy.
E.10. The method according to embodiment E.9, where R² is selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b};
   where each R^{b} is independently selected from the group consisting of halogen, cyano, nitro, C₁-C₄-alkyl, C₁-C₄-haloalkyl, C₁-C₄-alkoxy, C₁-C₄-haloalkoxy and phenyl; preferably from halogen, cyano, nitro, C₁-C₄-alkyl, C₁-C₄-haloalkyl, C₁-C₄-alkoxy and C₁-C₄-haloalkoxy; and m is 1, 2 or 3.
E.11. The method according to embodiment E.10, where R² is selected from the group consisting of phenyl which is unsubstituted or carries m radicals R^{b}, and 6- to 10-membered heteroaromatic ring containing 1 or 2 nitrogen atoms as ring members, which is unsubstituted or carries m radicals R^{b};
   where each R^{b} is independently selected from the group consisting of halogen, nitro, C₁-C₄-alkyl, C₁-C₄-alkoxy and phenyl; preferably from halogen, nitro, C₁-C₄-alkyl and C₁-C₄-alkoxy; and m is 1, 2 or 3.
E.12. The method according to any of the preceding embodiments, where R³ is hydrogen or C₁-C₄-alkyl, preferably hydrogen or methyl.
E.13. The method according to embodiment E.12, where R³ is hydrogen.
E.14. The method according to any of embodiments E.1 to E.11, where R³ forms a linking group —(CH₂)₂—, —(CH₂)₃— or —CH═CH— to a carbon or nitrogen ring atom of the aryl or heteroaromatic ring R².
E.15. The method according to embodiment E.14, where R³ forms a linking group —(CH₂)₂— to a carbon or nitrogen ring atom of the aryl or heteroaromatic ring R².
E.16. The method according to any of the preceding embodiments, where the linking group R³ is bound to the ring atom of R² neighbouring the ring atom which forms the attachment point to N (of the group NR²R³), the resulting ring thus being condensed to the (hetero)aromatic ring R².
E.17. The method according to any of embodiments E.1 to E.11 and E.14 to E.16, where the moiety —NR²R³ forms 2,3-dihydroindol-1-yl, 1,2,3,4-tetrahydroquinolin-1-yl or indol-1-yl.
E.18. The method according to embodiment E.17, where the moiety —NR²R³ forms 2,3-dihydroindol-1-yl.
E.19. The method according to any of the preceding embodiments, where A is C₁-C₈-alkanediyl, C₂-C₈-alkenediyl, C₃-C₆-cycloalkanediyl or phenylene.
E.20. The method according to embodiment E.19, where A is C₁-C₈-alkanediyl, preferably C₁-C₄-alkanediyl.
E.21. The method according to embodiment E.20, where A is —CH₂—.
E.22. The method according to any of the preceding embodiments, where R⁴ is selected from the group consisting of C₁-C₄-alkyl, phenyl and benzyl, preferably from C₁-C₄-alkyl and phenyl.
E.23. The method according to embodiment E.22, where R⁴ is C₁-C₄-alkyl.
E.24. The method according to embodiment E.23, where R⁴ is methyl or ethyl.
E.25. The method according to any of the preceding embodiments, where the compound of the formula (II-1) and the compound of the formula (III) are used in a molar ratio of from 5:1 to 1:5.
E.26. The method according to embodiment E.25, where the compound of the formula (II-1) and the compound of the formula (III) are used in a molar ratio of from 2:1 to 1:2.
E.27. The method according to embodiment E.26, where the compound of the formula (II-1) and the compound of the formula (III) are used in a molar ratio of from 1.5:1 to 1:1.5.
E.28. The method according to embodiment E.27, where the compound of the formula (II-1) and the compound of the formula (III) are used in a molar ratio of approximately 1:1.
E.29. The method according to any of the preceding embodiments, where the compound of the formula (II-2) and the compound of the formula (III) are used in a molar ratio of from 2.5:1 to 1:10.
E.30. The method according to embodiment E.29, where the compound of the formula (II-2) and the compound of the formula (III) are used in a molar ratio of from 1:1 to 1:4.
E.31. The method according to embodiment E.30, where the compound of the formula (II-2) and the compound of the formula (III) are used in a molar ratio of from 0.75:1 to 1:3.
E.32. The method according to embodiment E.31, where the compound of the formula (II-2) and the compound of the formula (III) are used in a molar ratio of approximately 1:2.
E.33. The method according to any of the preceding embodiments, where the Lewis acid is selected from the halides, nitrates, carboxylates of the formula R—COO⁻, where R is C₁-C₁₀-alkyl, C₃-C₆-cycloalkyl or C₃-C₆-cycloalkyl-C₁-C₁₀-alkyl (to be more precise carboxylate salts where the anion has the formula R—COO⁻, or alternatively expressed salts of carboxylic acids of the formula R—C(O)OH where R is C₁-C₁₀-alkyl, C₃-C₆-cycloalkyl or C₃-C₆-cycloalkyl-C₁-C₁₀-alkyl), acetylacetonates, C₁-C₄-alkoxides or carbonyl complexes of metals of groups 4, 6 to 10, 12, 13 and 15 of the periodic table of elements.
E.34. The method according to embodiment E.33, where the Lewis acid is selected from the halides, nitrates, carboxylates of the formula R—COO⁻, where R is C₁-C₁₀-alkyl, C₃-C₆-cycloalkyl or C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₁-C₁₀-alkyl, C₃-C₆-cycloalkyl or C₃-C₆-cycloalkyl-C₁-C₁₀-alkyl), acetylacetonates, C₁-C₄-alkoxides and carbonyl complexes of Ti, Zr, Hf, Cr, Mo, Mn, Fe, Co, Ni, Zn, Al, Sb or Bi.
E.35. The method according to embodiment E.34, where the Lewis acid is selected from the group consisting of the halides, nitrates, carboxylates of the formula R—COO⁻, where R is C₁-C₁₀-alkyl, C₃-C₆-cycloalkyl or C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₁-C₁₀-alkyl, C₃-C₆-cycloalkyl or C₃-C₆-cycloalkyl-C₁-C₁₀-alkyl), acetylacetonates, C₁-C₄-alkoxides and carbonyl complexes of Mn, Co, Zn or Bi.
E.36. The method according to embodiment E.35, where the Lewis acid is selected from the group consisting of the halides, the carboxylates of the formula R—COO⁻, where R is C₁-C₄-alkyl or C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₁-C₄-alkyl or C₃-C₆-cycloalkyl-C₁-C₁₀-alkyl), the acetylacetonates, the C₁-C₄-alkoxides and the carbonyl complexes of Mn, Co, Zn or Bi,
   where the Lewis acid is preferably selected from the group consisting of the halides of Mn, Co, Zn or Bi, the carboxylates of the formula R—COO⁻, where R is C₁-C₄-alkyl or C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₁-C₄-alkyl or C₃-C₆-cycloalkyl-C₁-C₁₀-alkyl) of Mn, Co, Zn or Bi, the acetylacetonates of Mn, Co, Zn or Bi, the C₁-C₄-alkoxides of Mn, Co, Zn or Bi and the carbonyl complexes of Mn.
E.37. The method according to embodiment E.34, where the Lewis acid is selected from the group consisting of MnCl₂, MoCl₃, CrCl₃, BiCl₃, SbCl₃, ZnCl₂, FeCl₃, FeCl₂, COCl₂, NiCl₂, TiCl₄, ZrCl₄, HfCl₄, MnBr₂, Mn(NO₃)₂, Co(NO₃)₂, Mn(OAc)₂, Mn(4-cyclohexylbutyrat)₂, Fe(OAc)₃, Bi(OAc)₃, Mn(AcAc)₂, Mn(AcAc)₃, Fe(AcAc)₂, Fe(AcAc)₃, Ni(AcAc)₂, Bi(OiPr)₃, Ti(OiPr)₄, AI(OiPr)₃, Mn₂(CO)₁₀, Mn(CO)₅Br, Cr(CO)₆, Fe(CO)₄ and CO₂(CO)₈; where OAc means acetate, AcAc means acetylacetonate and OiPr means isopropoxide.
E.38. The method according to embodiment E.37, where the Lewis acid is selected from the group consisting of MnCl₂, MnBr₂, BiCl₃, COCl₂, ZnCl₂, NiCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀.
E.39. The method according to embodiment E.38, where the Lewis acid is selected from the group consisting of MnCl₂, MnBr₂, BiCl₃, COCl₂, ZnCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀.
E.40. The method according to embodiment E.39, where the Lewis acid is selected from the group consisting of MnCl₂, MnBr₂, BiCl₃, COCl₂, ZnCl₂, Mn(OAc)₂, Mn(AcAc)₂, Mn(AcAc)₃, Bi(OiPr)₃ and Mn₂(CO)₁₀.
E.41. The method according to embodiment E.40, where the Lewis acid is selected from the group consisting of MnCl₂, MnBr₂, BiCl₃, COCl₂, Mn(AcAc)₃, Bi(OiPr)₃ and Mn₂(CO)₁₀.
E.42. The method according to embodiment E.41, where the Lewis acid is selected from the group consisting of MnCl₂ and MnBr₂.
E.43. The method according to any of the preceding embodiments, where the Lewis acid is used in an amount of from 0.00001 to 0.2 mol per mol of that compound (II) or (III) which is not used in excess.
E.44. The method according to embodiment E.43, where the Lewis acid is used in an amount of from 0.00001 to 0.1 mol per mol of that compound (II) or (III) which is not used in excess.
E.45. The method according to embodiment E.44, where the Lewis acid is used in an amount of from 0.0001 to 0.05 mol per mol of that compound (II) or (III) which is not used in excess.
E.46. The method according to embodiment E.45, where the Lewis acid is used in an amount of from 0.001 to 0.01 per mol of that compound (II) or (III) which is not used in excess.
E.47. The method according to embodiment E.45, where the Lewis acid is used in an amount of from 0.005 to 0.009 mol per mol of that compound (II) or (III) which is not used in excess.
E.48. The method according to any of the preceding embodiments, where the alkali metal-containing base is selected from the group consisting of alkali metal alkoxides, amides, hydrides, borohydrides and aluminiumhydrides.
E.49. The method according to embodiment E.48, where the alkali metal-containing base is selected from the group consisting of alkali metal C₁-C₁₀-alkoxides, alkali metal amides of the formula M⁺[N(R^{g})₂]⁻, where M⁺ is an alkali metal cation and R^{g} is hydrogen, C₁-C₄-alkyl or Si(C₁-C₄-alkyl)₂; and alkali metal borohydrides of the formula M⁺[BH(C₁-C₄-alkyl)₃]⁻, where M⁺ is an alkali metal cation.
E.50. The method according to any of the preceding embodiments, where the alkali metal-containing base is used in an amount of from 0.00001 to 0.1 mol per mol of that compound (II) or (III) which is not used in excess.
E.51. The method according to embodiment E.50, where the alkali metal-containing base is used in an amount of from 0.0001 to 0.05 mol per mol of that compound (II) or (III) which is not used in excess.
E.52. The method according to embodiment E.51, where the alkali metal-containing base is used in an amount of from 0.001 to 0.04 mol per mol of that compound (II) or (III) which is not used in excess.
E.53. The method according to embodiment E.52, where the alkali metal-containing base is used in an amount of from 0.01 to 0.04 mol per mol of that compound (II) or (III) which is not used in excess.
E.54. The method according to any of the preceding embodiments, where the water content in the reaction mixture is at most 0.1% by weight, relative to the total weight of the reaction mixture.
E.55. The method according to embodiment E.54, where the water content in the reaction mixture is less than 0.1% by weight, relative to the total weight of the reaction mixture.
E.56. The method according to embodiment E.55, where the water content in the reaction mixture is less than 0.08% by weight, relative to the total weight of the reaction mixture.
E.57. The method according to any of the preceding embodiments, where the reaction is carried out under continuous removal of the alcohol R⁴—OH formed during the reaction.
E.58. The method according to any of the preceding embodiments, where the reaction is carried out at a temperature of from 80 to 180° C.
E.59. The method according to embodiment E.58, where the reaction is carried out at a temperature of from 90 to 160° C.
E.60. The method according to embodiment E.59, where the reaction is carried out at a temperature of from 100 to 160° C.
E.61. The method according to embodiment E.60, where the reaction is carried out at a temperature of from 120 to 160° C.

Schematically, the amidation reaction can be depicted as follows:

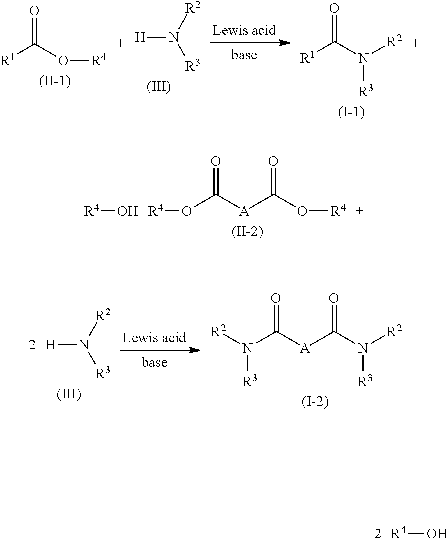

In compounds (I-1) and (II-1), R¹ is preferably selected from the group consisting of C₁-C₂₀-alkyl, C₁-C₄-alkyl which carries 1 or 2 radicals R^{a}, C₂-C₂₀-alkenyl, C₂-C₄-alkenyl which carries a phenyl ring, C₃-C₆-cycloalkyl which is unsubstituted or carries m radicals R^{b}, C₆-C₁₀-aryl which is unsubstituted or carries m radicals R^{b}, and a 5- to 10-membered heteroaromatic ring containing 1, 2, 3 or 4 heteroatoms selected from, N, O and S as ring members, which is unsubstituted or carries m radicals R^{b};
where
each R^{a} is independently C₁-C₄-alkoxy, C₁-C₄-haloalkoxy, C(═O)R^{c} or phenyl; and
each R^{b} is independently halogen, cyano, C₁-C₄-alkyl, C₁-C₄-haloalkyl, C₁-C₄-alkoxy, C₁-C₄-haloalkoxy and a 5- or 6-membered heteroaromatic ring containing 1, 2, 3 or 4 heteroatoms selected from, N, O and S as ring members, which is unsubstituted or carries m radicals R^{d}; where each R^{d} is independently selected from the group consisting of halogen, cyano, C₁-C₄-alkyl, C₁-C₄-haloalkyl, C₁-C₄-alkoxy and C₁-C₄-haloalkoxy.

More preferably, R¹ is selected from the group consisting of C₁-C₂₀-alkyl, C₁-C₄-alkyl which carries a phenyl ring, C₂-C₂₀-alkenyl, C₂-C₄-alkenyl which carries a phenyl ring, C₆-C₁₀-aryl which is unsubstituted or carries m radicals R^{b}, and a 5- to 10-membered heteroaromatic ring containing 1, 2, 3 or 4 heteroatoms selected from, N, O and S as ring members, which is unsubstituted or carries m radicals R^{b};
where each R^{b} is independently halogen, cyano, C₁-C₄-alkyl, C₁-C₄-haloalkyl, C₁-C₄-alkoxy and C₁-C₄-haloalkoxy.

Even more preferably, R¹ is selected from the group consisting of C₁-C₄-alkyl which carries a phenyl ring, C₂-C₂₀-alkenyl, C₂-C₄-alkenyl which carries a phenyl ring, phenyl which is unsubstituted or carries m radicals R^{b}, and a 5- or 6-membered heteroaromatic ring containing 1, 2 or 3 heteroatoms selected from, N, O and S as ring members, which is unsubstituted or carries m radicals R^{b};
where each R^{b} is independently halogen, cyano, C₁-C₄-alkyl and C₁-C₄-alkoxy; and m is 1, 2 or 3.

Particularly preferably, R¹ is selected from the group consisting of C₁-C₂-alkyl which carries a phenyl ring, C₂-C₂₀-alkenyl, C₂-alkenyl which carries a phenyl ring, phenyl which is unsubstituted or carries m radicals R^{b}, and a 5- or 6-membered heteroaromatic ring containing 1 or 2 nitrogen atoms as ring members (e.g. pyridyl, pyrimidyl, pyrazinyl, pyridazinyl, pyrrolyl, pyrazolyl or imidazolyl; specifically pyridyl or pyrazolyl), which is unsubstituted or carries m radicals R^{b};
where each R^{b} is independently halogen, cyano, C₁-C₄-alkyl, C₁-C₄-haloalkyl, C₁-C₄-alkoxy and C₁-C₄-haloalkoxy; and m is 1, 2 or 3.

In particular, R¹ is selected from the group consisting of C₁-C₂-alkyl which carries a phenyl ring, C₂-C₂₀-alkenyl, C₂-alkenyl which carries a phenyl ring, phenyl which is unsubstituted or carries m radicals R^{b}, and pyridyl which is unsubstituted or carries m radicals R^{b};
where each R^{b} is independently halogen, cyano, C₁-C₄-alkyl and C₁-C₄-alkoxy; and m is 1, 2 or 3.

More particularly, R¹ is phenyl which is unsubstituted or carries m radicals R^{b}, or a 5- or 6-membered heteroaromatic ring containing 1, 2 or 3 heteroatoms selected from, N, O and S as ring members, which is unsubstituted or carries m radicals R^{b};
where each R^{b} is independently halogen, cyano, C₁-C₄-alkyl, C₁-C₄-haloalkyl, C₁-C₄-alkoxy and C₁-C₄-haloalkoxy; and m is 1, 2 or 3.

Even more particularly, R¹ is phenyl which is unsubstituted or carries m radicals R^{b}, or a 5- or 6-membered heteroaromatic ring containing 1 or 2 nitrogen atoms as ring members, which is unsubstituted or carries m radicals R^{b}.
where each R^{b} is independently halogen, cyano, C₁-C₄-alkyl, C₁-C₄-haloalkyl, C₁-C₄-alkoxy and C₁-C₄-haloalkoxy; and m is 1, 2 or 3.

Very particularly, R¹ is phenyl which is unsubstituted or carries m radicals R^{b}, or pyridyl which is unsubstituted or carries m radicals R^{b}; where each R^{b} is independently halogen, cyano, C₁-C₄-alkyl and C₁-C₄-alkoxy; and m is 1, 2 or 3.

In compounds (I-1), (I-2) and (III), R² is preferably selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b};
where
each R^{b} is independently selected from the group consisting of halogen, cyano, hydroxyl, nitro, C(═O)NR^{e}R^{f}, C₁-C₄-alkyl, C₁-C₄-haloalkyl, C₁-C₄-alkoxy, C₁-C₄-haloalkoxy and phenyl which is unsubstituted or carries m radicals R^{d};
each R^{d} is independently selected from the group consisting of halogen, C₁-C₄-alkyl and C₁-C₄-haloalkyl;
each R^{e} is independently selected from the group consisting of hydrogen and C₁-C₄-alkyl;
each R^{f} is independently selected from the group consisting of —C(═O)-phenyl and phenyl which is unsubstituted or substituted by 1, 2 or 3 radicals selected from the group consisting of halogen, cyano, C₁-C₄-alkyl, C₁-C₄-haloalkyl, C₁-C₄-alkoxy and C₁-C₄-haloalkoxy.

More preferably, R² is selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b}; where each R^{b} is independently selected from the group consisting of halogen, cyano, nitro, C₁-C₄-alkyl, C₁-C₄-haloalkyl, C₁-C₄-alkoxy, C₁-C₄-haloalkoxy and phenyl; preferably from halogen, cyano, nitro, C₁-C₄-alkyl, C₁-C₄-haloalkyl, C₁-C₄-alkoxy and C₁-C₄-haloalkoxy; and m is 1, 2 or 3.

Even more preferably, R² is selected from the group consisting of phenyl which is unsubstituted or carries m radicals R^{b}, and 6- to 10-membered heteroaromatic ring containing 1 or 2, preferably 1, nitrogen atoms as ring members (e.g. pyridyl or quinolinyl), which is unsubstituted or carries m radicals R^{b}; where each R^{b} is independently selected from the group consisting of halogen, nitro, C₁-C₄-alkyl, C₁-C₄-alkoxy and phenyl; preferably halogen, nitro, C₁-C₄-alkyl and C₁-C₄-alkoxy; and m is 1, 2 or 3.

In compounds (I-1), (I-2) and (III), R³ is preferably hydrogen or C₁-C₄-alkyl, more preferably hydrogen or methyl, even more preferably hydrogen.

Alternatively, R³ forms a saturated or unsaturated 2-, 3- or 4-membered linking group to a carbon or nitrogen ring atom of the aryl or heteroaromatic ring R²; where the linking group may comprise 1 or 2 heteroatoms or heteroatom groups selected from, N, O, S, SO and SO₂. As explained above, this results in a bicyclic heterocyclic ring system if R² is monocyclic or in a polycyclic heterocyclic ring system if R² is bicyclic or polycyclic, where the resulting ring system contains the nitrogen atom of NR²R³ as ring member and is bound via this nitrogen ring member to CO. If the linking group R³ is bound to the ring atom of R² neighbouring the ring atom which forms the attachment point to N (of the group NR²R³), the resulting ring is condensed to the (hetero)aromatic ring R². If the linking group R³ is bound to another ring atom of R², the resulting ring system is a bridged one.

In an alternatively preferred embodiment, R³ forms a linking group —(CH₂)₂—, —(CH₂)₃— or —CH═CH— to a carbon or nitrogen ring atom of the aryl or heteroaromatic ring R², preferably a linking group —(CH₂)₂—. Preferably, the linking group R³ is bound to the ring atom of R² neighbouring the ring atom which forms the attachment point to N (of the group NR²R³), the resulting ring thus being condensed to the (hetero)aromatic ring R².

The thusly resulting ring system —NR²R³ is preferably 2,3-dihydroindol-1-yl, 1,2,3,4-tetrahydroquinolin-1-yl or indol-1-yl and more preferably 2,3-dihydroindol-1-yl.

In compounds (I-2) and (II-2), A is preferably C₁-C₈-alkanediyl, C₂-C₈-alkenediyl, C₃-C₆-cycloalkanediyl or phenylene; more preferably C₁-C₈-alkanediyl, even more preferably C₁-C₆-alkanediyl, particularly preferably C₁-C₄-alkanediyl, and in particular —CH₂—.

Compounds (II-2) are non-activated esters.

R⁴ is preferably selected from the group consisting of C₁-C₄-alkyl, phenyl and benzyl; and is more preferably C₁-C₄-alkyl or phenyl, even more preferably C₁-C₄-alkyl, and is in particular methyl or ethyl.

In a particular embodiment, in compounds (I-1)
R¹ is phenyl which is unsubstituted or carries m radicals R^{b}, or a 5- or 6-membered heteroaromatic ring containing 1, 2 or 3 heteroatoms selected from, N, O and S as ring members, which is unsubstituted or carries m radicals R^{b}; where each R^{b} is independently selected from the group consisting halogen, cyano, C₁-C₄-alkyl, C₁-C₄-haloalkyl, C₁-C₄-alkoxy and C₁-C₄-haloalkoxy; and m is 1, 2 or 3;
R² is selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b}; where each R^{b} is independently selected from the group consisting halogen, cyano, nitro, C₁-C₄-alkyl, C₁-C₄-haloalkyl, C₁-C₄-alkoxy and C₁-C₄-haloalkoxy; and m is 1, 2 or 3; and
R³ is hydrogen or C₁-C₄-alkyl; or
—NR²R³ is 2,3-dihydroindol-1-yl, 1,2,3,4-tetrahydroquinolin-1-yl or indol-1-yl.

More particularly, in compounds (I-1)
R¹ is phenyl which is unsubstituted or carries m radicals R^{b}, or a 5- or 6-membered heteroaromatic ring containing 1 or 2 nitrogen atoms as ring members, which is unsubstituted or carries m radicals R^{b}; where each R^{b} is independently selected from the group consisting halogen, cyano, C₁-C₄-alkyl, C₁-C₄-haloalkyl, C₁-C₄-alkoxy and C₁-C₄-haloalkoxy; and m is 1, 2 or 3;
R² is selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b}; where each R^{b} is independently selected from the group consisting halogen, cyano, nitro, C₁-C₄-alkyl, C₁-C₄-haloalkyl, C₁-C₄-alkoxy and C₁-C₄-haloalkoxy; and m is 1, 2 or 3; and
R³ is hydrogen or C₁-C₄-alkyl; or
—NR²R³ is 2,3-dihydroindol-1-yl, 1,2,3,4-tetrahydroquinolin-1-yl or indol-1-yl.

Even more particularly, in compounds (I-1)
R¹ is phenyl which is unsubstituted or carries m radicals R^{b}, or pyridyl which is unsubstituted or carries m radicals R^{b}; where each R^{b} is independently halogen, cyano, C₁-C₄-alkyl or C₁-C₄-alkoxy; and m is 1, 2 or 3;
R² is selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b}; where each R^{b} is independently selected from the group consisting halogen, cyano, nitro, C₁-C₄-alkyl, C₁-C₄-haloalkyl, C₁-C₄-alkoxy and C₁-C₄-haloalkoxy; and m is 1, 2 or 3; and
R³ is hydrogen or C₁-C₄-alkyl; or
—NR²R³ is 2,3-dihydroindol-1-yl, 1,2,3,4-tetrahydroquinolin-1-yl or indol-1-yl.

Very particularly, in compounds (I-1)
R¹ is phenyl which is unsubstituted or carries m radicals R^{b}, or pyridyl which is unsubstituted or carries m radicals R^{b}; where each R^{b} is independently halogen, cyano, C₁-C₄-alkyl and C₁-C₄-alkoxy; and m is 1, 2 or 3;
R² is selected from the group consisting of phenyl which is unsubstituted or carries m radicals R^{b}, and a 6- to 10-membered heteroaromatic ring containing 1 or 2 nitrogen atoms as ring members, which is unsubstituted or carries m radicals R^{b}; where each R^{b} is independently selected from the group consisting halogen, nitro, C₁-C₄-alkyl and C₁-C₄-alkoxy; and m is 1, 2 or 3; and
R³ is hydrogen; or
—NR²R³ is 2,3-dihydroindol-1-yl.

In particular, in compounds (I-2)
A is C₁-C₆-alkanediyl;
R² is selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b}; where each R^{b} is independently selected from the group consisting halogen, cyano, nitro, C₁-C₄-alkyl, C₁-C₄-haloalkyl, C₁-C₄-alkoxy and C₁-C₄-haloalkoxy; and m is 1, 2 or 3; and
R³ is hydrogen or C₁-C₄-alkyl; or
—NR²R³ is 2,3-dihydroindol-1-yl, 1,2,3,4-tetrahydroquinolin-1-yl or indol-1-yl.

More particularly, in compounds (I-2)
A is —CH₂—;
R² is selected from the group consisting of phenyl which is unsubstituted or carries m radicals R^{b}, and a 6- to 10-membered heteroaromatic ring containing 1 or 2 nitrogen atoms as ring members, which is unsubstituted or carries m radicals R^{b}; where each R^{b} is independently selected from the group consisting halogen, nitro, C₁-C₄-alkyl and C₁-C₄-alkoxy; and m is 1, 2 or 3; and
R³ is hydrogen.

In particular, in compounds (II-1)
R¹ is phenyl which is unsubstituted or carries m radicals R^{b}, or a 5- or 6-membered heteroaromatic ring containing 1, 2 or 3 heteroatoms selected from, N, O and S as ring members, which is unsubstituted or carries m radicals R^{b}; where each R^{b} is independently selected from the group consisting halogen, cyano, C₁-C₄-alkyl, C₁-C₄-haloalkyl, C₁-C₄-alkoxy and C₁-C₄-haloalkoxy; and m is 1, 2 or 3; and
R⁴ is C₁-C₄-alkyl, phenyl or benzyl; preferably C₁-C₄-alkyl or phenyl.

More particularly, in compounds (II-1)
R¹ is phenyl which is unsubstituted or carries m radicals R^{b}, or a 5- or 6-membered heteroaromatic ring containing 1 or 2 nitrogen atoms as ring members, which is unsubstituted or carries m radicals R^{b}; where each R^{b} is independently selected from the group consisting halogen, cyano, C₁-C₄-alkyl, C₁-C₄-haloalkyl, C₁-C₄-alkoxy and C₁-C₄-haloalkoxy; and m is 1, 2 or 3;
and
R⁴ is C₁-C₄-alkyl, phenyl or benzyl; preferably C₁-C₄-alkyl or phenyl.

Even more particularly, in compounds (II-1)
R¹ is phenyl which is unsubstituted or carries m radicals R^{b}, or pyridyl which is unsubstituted or carries m radicals R^{b}; where each R^{b} is independently halogen, cyano, C₁-C₄-alkyl and C₁-C₄-alkoxy; and m is 1, 2 or 3; and
R⁴ is C₁-C₄-alkyl, phenyl or benzyl; preferably C₁-C₄-alkyl or phenyl.

Very particularly, in compounds (II-1)
R¹ is phenyl which is unsubstituted or carries m radicals R^{b}, or pyridyl which is unsubstituted or carries m radicals R^{b}; where each R^{b} is independently halogen, cyano, C₁-C₄-alkyl and C₁-C₄-alkoxy; and m is 1, 2 or 3; and
R⁴ is C₁-C₄-alkyl, preferably methyl or ethyl, specifically methyl.

In particular, in compounds (II-2)
A is C₁-C₆-alkanediyl; and
R⁴ is C₁-C₄-alkyl, phenyl or benzyl; preferably C₁-C₄-alkyl or phenyl.

More particularly, in compounds (II-2)
A is —CH₂—; and
R⁴ is C₁-C₄-alkyl, preferably methyl or ethyl, specifically methyl.

In particular, in compounds (III)
R² is selected from the group consisting of C₆-C₁₀-aryl which is unsubstituted or carries m radicals R^{b}, and a 5- to 10-membered heteroaromatic ring containing 1, 2, 3 or 4 heteroatoms selected from N, O and S as ring members, which is unsubstituted or carries m radicals R^{b}; where each R^{b} is independently selected from the group consisting halogen, cyano, nitro, C₁-C₄-alkyl, C₁-C₄-haloalkyl, C₁-C₄-alkoxy and C₁-C₄-haloalkoxy; and m is 1, 2 or 3; and
R³ is hydrogen or C₁-C₄-alkyl; or
—NR²R³ is 2,3-dihydroindol-1-yl, 1,2,3,4-tetrahydroquinolin-1-yl or indol-1-yl.

More particularly, in compounds (III)
R² is selected from the group consisting of phenyl which is unsubstituted or carries m radicals R^{b}, and a 6- to 10-membered heteroaromatic ring containing 1 or 2 nitrogen atoms as ring members, which is unsubstituted or carries m radicals R^{b}; where each R^{b} is independently selected from the group consisting halogen, nitro, C₁-C₄-alkyl and C₁-C₄-alkoxy; and m is 1, 2 or 3; and
R³ is hydrogen; or
—NR²R³ is 2,3-dihydroindol-1-yl.

In another particular embodiment,
R¹ is selected from the group consisting of C₁-C₂-alkyl which carries a phenyl ring, C₂-C₂₀-alkenyl, C₂-alkenyl which carries a phenyl ring, phenyl which is unsubstituted or carries m radicals R^{b}, and a 5- or 6-membered heteroaromatic ring containing 1 or 2 nitrogen atoms as ring members, which is unsubstituted or carries
   m radicals R^{b};
   where
   each R^{b} is independently halogen, cyano, C₁-C₄-alkyl, C₁-C₄-haloalkyl, C₁-C₄-alkoxy and C₁-C₄-haloalkoxy; and
   m is 1, 2 or 3;
R² is selected from the group consisting of phenyl which is unsubstituted or carries m radicals R^{b}, and 6- to 10-membered heteroaromatic ring containing 1 or 2 nitrogen atoms as ring members, which is unsubstituted or carries m radicals R^{b};
   where
   each R^{b} is independently selected from the group consisting halogen, nitro, C₁-C₄-alkyl, C₁-C₄-alkoxy and phenyl; and
   m is 1, 2 or 3;
R³ is hydrogen or C₁-C₄-alkyl;
or
—NR²R³ stands for 2,3-dihydroindol-1-yl, 1,2,3,4-tetrahydroquinolin-1-yl or indol-1-yl;
A is C₁-C₄-alkanediyl; and
R⁴ is C₁-C₄-alkyl or phenyl;

More particularly,
R¹ is selected from the group consisting of C₁-C₂-alkyl which carries a phenyl ring, C₂-C₂₀-alkenyl, C₂-alkenyl which carries a phenyl ring, phenyl which is unsubstituted or carries m radicals R^{b}, and a 5- or 6-membered heteroaromatic ring containing 1 or 2 nitrogen atoms as ring members, which is unsubstituted or carries m radicals R^{b};
   where
   each R^{b} is independently halogen, cyano, C₁-C₄-alkyl, C₁-C₄-haloalkyl, C₁-C₄-alkoxy and C₁-C₄-haloalkoxy; and
   m is 1, 2 or 3;
R² is selected from the group consisting of phenyl which is unsubstituted or carries m radicals R^{b}, pyridyl and quinolinyl;
   where
   each R^{b} is independently selected from the group consisting halogen, nitro, C₁-C₄-alkyl, C₁-C₄-alkoxy and phenyl; and
   m is 1, 2 or 3;
R³ is hydrogen;
or
—NR²R³ stands for 2,3-dihydroindol-1-yl;
A is —CH₂—; and
R⁴ is C₁-C₄-alkyl.

Compounds (II) and (III) are commercially available or can be prepared by known methods of organic chemistry.

Preferably, the compound of the formula (II-1) and the compound of the formula (III) are used in a molar ratio of from 5:1 to 1:5, more preferably from 2:1 to 1:2, even more preferably from 1.5:1 to 1:1.5 and in particular of approximately 1:1.

The compound of the formula (II-2) and the compound of the formula (III) are preferably used in a molar ratio of from 2.5:1 to 1:10, more preferably from 1:1 to 1:4, even more preferably from 0.75:1 to 1:3 and in particular of approximately 1:2.

“Approximately” in this context is intended to include deviations from ideal stoichiometry caused, for example, by weighing errors. Such errors are in general below 10%, mostly below 5% or even below 2%.

Among compounds (I-1) and (I-2) as well as (II-1) and (II-2), preference is given to (I-1) and (II-1).

The Lewis acid is preferably a metal salt or a metal complex. Preferably, the metal salt or complex is a salt or complex of a metal of groups 4, 6 to 10, 12, 13 and 15 of the periodic table of elements. More preferably, the metal salt or complex is selected from the halides, nitrates, carboxylates of the formula (of the anion) R—COO⁻, where R is C₁-C₁₀-alkyl, C₃-C₆-cycloalkyl or C₃-C₆-cycloalkyl-C₁-C₁₀-alkyl; acetylacetonates, C₁-C₄-alkoxides or carbonyl complexes of metals of groups 4, 6 to 10, 12, 13 and 15 of the periodic table of elements.

The group numbering relates to the IUPAC nomenclature of 1985. Groups 4, 6 to 10, 12, 13 and 15 are thus the Ti, Cr, Mn, Fe, Co, Ni, Zn, B and N groups. Within the N group (groups 15), only the metals, i.e. As, Sb, Bi, are meant. Within the B group (group 13), boron is included, although it is a semi-metal. Within group 13, preference is however given to the metals thereof, i.e. to Al, Ga, In, Tl.

Suitable halides are the fluorides, chlorides, bromides and iodides, preference being given to the chlorides and bromides.

The term “carboxylate” in context with the present Lewis acids refers to anions R—COO⁻, where R is an organic radical; in the present case a C₁-C₁₀-alkyl, C₃-C₆-cycloalkyl or C₃-C₆-cycloalkyl-C₁-C₁₀-alkyl group. The term “carboxylate” is however also used as pars pro toto to designate the salts of these anions. Carboxylates of the above-listed metals are thus salts of said metals, where the anion is of the formula R—COO⁻, where R is as defined above. Examples for suitable carboxylates are acetate, propionate, butyrate, isobutyrate, cyclohexylcarboxylate or 4-cyclohexylbutyrate (cyclohexyl-(CH₂)₃—C(O)O⁻).

C₁-C₄-Alkoxides are anions R—O⁻, where R is C₁-C₄-alkyl. The term “alkoxide” is however also used as pars pro toto to designate the salts of these anions. C₁-C₄-Alkoxides of the above-listed metals are thus salts of said metals, where the anion is of the formula R—O⁻, R being C₁-C₄-alkyl. Examples for suitable alkoxides are methoxide (methanolate; CH₃—O⁻), ethoxide (ethanolate; CH₃CH₂—O⁻), n-propoxide (n-propanolate; CH₃CH₂CH₂—O⁻), isopropoxide (isopropanolate CH(CH₃)₂—O⁻), n-butoxide (n-butanolate), sec-butoxide (sec-butanolate), isobutoxide (isobutanolate) or tert-butoxide (tert-butanolate).

Suitable carbonyl complexes are those of the above-mentioned transition metals of groups 4, 6 to 10 and 12. If in the carbonyl complex the metal has noble gas configuration, the complex has to be sufficiently labile to allow dissociation (lose of a CO and/or dissociation of a binuclear complex) under the reaction conditions so as to form a fragment with Lewis acidity. Examples for suitable carbonyl complexes are Mn(CO)₅Br, Cr(CO)₆, Fe(CO)₄ and the binuclear complexes Mn₂(CO)₁₀ and CO₂(CO)₈.

Even more preferably, the metal salt or complex is a salt or complex of a metal of Ti, Zr, Hf, Cr, Mo, Mn, Fe, Co, Ni, Zn, Al, Sb and Bi. Among the salts and complexes of said metals, preference is given to the halides, nitrates, carboxylates of the formula R—COO⁻, where R is C₁-C₁₀-alkyl (to be more precise carboxylate salts where the anion has the formula R—COO⁻, or alternatively expressed salts of carboxylic acids of the formula R—C(O)OH, where R is C₁-C₁₀-alkyl); carboxylates of the formula R—COO⁻, where R is C₃-C₆-cycloalkyl (to be more precise carboxylate salts where the anion has the formula R—COO⁻, or alternatively expressed salts of carboxylic acids of the formula R—C(O)OH, where R is C₃-C₆-cycloalkyl); carboxylates of the formula R—COO⁻, where R is C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₃-C₆-cycloalkyl-C₁-C₁₀-alkyl); acetylacetonates, C₁-C₄-alkoxides or carbonyl complexes of Ti, Zr, Hf, Cr, Mo, Mn, Fe, Co, Ni, Zn, Al, Sb and Bi. More preference is given to MnCl₂, MoCl₃, CrCl₃, BiCl₃, SbCl₃, ZnCl₂, FeCl₃, FeCl₂, COCl₂, NiCl₂, TiCl₄, ZrCl₄, HfCl₄, MnBr₂, Mn(NO₃)₂, Co(NO₃)₂, Mn(OAc)₂, Mn(4-cyclohexylbutyrate)₂, Fe(OAc)₃, Bi(OAc)₃, Mn(AcAc)₂, Mn(AcAc)₃, Fe(AcAc)₂, Fe(AcAc)₃, Ni(AcAc)₂, Bi(OiPr)₃, Ti(OiPr)₄, AI(OiPr)₃, Mn₂(CO)₁₀, Mn(CO)₅Br, Cr(CO)₆, Fe(CO)₄ and CO₂(CO)₈; where OAc means acetate, AcAc means acetylacetonate and OiPr means isopropoxide.

Particular preference is given to Lewis acids selected from the group consisting of the halides, the carboxylates of the formula R—COO⁻, where R is C₁-C₄-alkyl (to be more precise carboxylate salts where the anion has the formula R—COO⁻, or alternatively expressed salts of carboxylic acids of the formula R—C(O)OH, where R is C₁-C₄-alkyl); the carboxylates of the formula R—COO⁻, where R is C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₃-C₆-cycloalkyl-C₁-C₁₀-alkyl), the acetylacetonates, the C₁-C₄-alkoxides and the carbonyl complexes of Mn, Co, Zn or Bi; especially the halides, the carboxylates of the formula R—COO⁻, where R is C₁-C₄-alkyl (to be more precise carboxylate salts where the anion has the formula R—COO⁻, or alternatively expressed salts of carboxylic acids of the formula R—C(O)OH, where R is C₁-C₄-alkyl); the carboxylates of the formula R—COO⁻, where R is C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₃-C₆-cycloalkyl-C₁-C₁₀-alkyl); the acetylacetonates and the C₁-C₄-alkoxides of Mn, Bi, Co, Zn or Ni; and to carbonyl complexes of Mn; more particular preference being given to Lewis acids selected from the group consisting of MnCl₂, MnBr₂, BiCl₃, COCl₂, ZnCl₂, NiCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀.

Even more particular preference is given to Lewis acids selected from the group consisting of MnCl₂, MnBr₂, BiCl₃, COCl₂, ZnCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀.

In particular, the Lewis acid is selected from the group consisting of MnCl₂, MnBr₂, BiCl₃, COCl₂, ZnCl₂, Mn(OAc)₂, Mn(AcAc)₂, Mn(AcAc)₃, Bi(OiPr)₃ and Mn₂(CO)₁₀; and specifically from MnCl₂ and MnBr₂.

Mixtures of two or more Lewis acids can also be used.

The Lewis acids are generally used in essentially anhydrous form; in the case of the metal salts this means essentially without crystal water. “Essentially” in this context means that some negligible amounts of water may be present, i.e. at most 5% by weight, preferably at most 2% by weight and more preferably at most 1% by weight, e.g. at most 0.1% by weight, relative to the total weight of the Lewis acid (metal salt or complex).

Thus, the reaction being carried out under anhydrous conditions preferably means that the water content in the reaction mixture, inclusive water contained as crystal water, is at most 0.15% by weight, relative to the total weight of the reaction mixture. The reaction medium is generally composed of the starting materials (II) and (III), the Lewis acid(s), the base(s) and optionally one or more solvents.

The Lewis acids are commercially available or can be prepared by known means from suitable starting materials, e.g. by anion exchange of a commercially available salt.

While the Lewis acid can principally be used in stoichiometric amounts or even in excess (relative to the starting materials), the present method advantageously works also very well when very low, substoichiometric amounts of the Lewis acid are used. Thus, the Lewis acid is preferably used in an amount of from 0.00001 to 0.2 mol, more preferably from 0.00001 to 0.1 mol, even more preferably from 0.0001 to 0.05 mol, particularly preferably from 0.001 to 0.01 mol, and in particular from 0.005 to 0.009 mol per mol of that compound (II) or (III) which is not used in excess. Alternatively expressed, the Lewis acid is used in an amount of preferably from 0.001 to 20 mol-%, more preferably from 0.001 to 10 mol-%, even more preferably from 0.01 to 5 mol-%, particularly preferably from 0.1 to 1 mol-%, and in particular from 0.5 to 0.9 mol-%, relative to the amount (in mol; this amount corresponds to 100 mol-%) of that compound (II) or (III) which is not used in excess.

The compound (II) or (III) “which is not used in excess” relates to that starting compound (II) and (III) which among compounds (II) and (III) is not used in excess. If compounds (II) or (III) are used in equimolar amounts, of course none of (II) and (III) is used in excess among these two compounds.

If compounds (II) or (III) are used in equimolar amounts, the above-given amounts of Lewis acid relate of course to either one of compounds (II) or (III).

In case of compounds (II-2), “equimolar amounts” and “excess” amounts take account in this specific context of the two carboxylate groups (carboxylate groups meaning in this context carboxylic ester groups) which can react in the amidation reaction. Thus, “equimolar” amounts of (II-2) and (III) means in this case 0.5 mol of (II-2) per 1 mol of (III); (II-2) used “in excess” means in this case >0.5 mol of (II-2) per 1 mol of (III), and (III) used “in excess” means in this case >2 mol of (III) per 1 mol of (II-2).

The alkali metal-containing base is preferably selected from the group consisting of alkali metal alkoxides, amides, hydrides, borohydrides and aluminiumhydrides.

Suitable alkali metals are Li, Na, K, R^{b} and Cs, preference being given to Li, Na, K and Cs.

Alkoxides are anions R—O⁻, where R is an alkyl group, preferably C₁-C₁₀-alkyl. Alkali metal alkoxides are thus salts R—O-M⁺, where M⁺ is an alkali metal cation. Examples for suitable C₁-C₁₀-alkoxide anions are methoxide (methanolate; CH₃—O⁻), ethoxide (ethanolate; CH₃CH₂—O⁻), n-propoxide (n-propanolate; CH₃CH₂CH₂—O⁻), isopropoxide (isopropanolate CH(CH₃)₂—O⁻), n-butoxide (n-butanolate), sec-butoxide (sec-butanolate), isobutoxide (isobutanolate), tert-butoxide (tert-butanolate), pentoxide, hexoxide, heptoxide, octoxide, 2-ethylhexoxide, nonoxide, decoxide, 2-propylheptoxide or other positional isomers thereof. Examples for suitable alkali metal C₁-C₁₀-alkoxides are LiOMe, NaOMe, KOMe, CsOMe, LiOEt, NaOEt, KOEt, CsOEt, LiOPr, NaOPr, KOPr, CsOPr, LiOiPr, NaOiPr, KOiPr, CsOiPr, LiOBu, NaOBu, KOBu, CsOBu, LiOtBu, NaOtBu, KOtBu, CsOtBu, LiO(2-ethylhexyl), NaO(2-ethylhexyl), KO(2-ethylhexyl), CsO(2-ethylhexyl) and the like, where OMe is methoxide, OEt is ethoxide, OPr is n-propoxide, OiPr is isopropoxide, OBu is n-butoxide, OtBu is tert-butoxide and O(2-ethylhexyl) is 2-ethylhexoxide.

Suitable amides are anions of the formula [N(R^{g})₂]⁻, where R^{g} is hydrogen, alkyl (generally C₁-C₄-alkyl) or Si(alkyl)₂ (generally Si(C₁-C₄-alkyl)₂). Generally, the two R^{g} in said amide anion have the same meaning. Alkali metal amides are thus salts of the formula M⁺[N(R^{g})₂]⁻, where M⁺ is an alkali metal cation. Examples for suitable alkali metal amides are LiNH₂, NaNH₂, KNH₂, CsNH₂, LiN(CH₃)₂, NaN(CH₃)₂, KN(CH₃)₂, CsN(CH₃)₂, LiN(CH₂CH₃)₂, NaN(CH₂CH₃)₂, KN(CH₂CH₃)₂, CsN(CH₂CH₃)₂, LiN(Si(CH₃)₃)₂, NaN(Si(CH₃)₃)₂ (NaHMDS) KN(Si(CH₃)₃)₂ (KHMDS), CsN(Si(CH₃)₃)₂ and the like.

Alkali metal hydrides are salts of the formula M⁺H⁻, where M⁺ is an alkali metal cation. Examples are LiH, NaH and KH.

Suitable alkali metal borohydrides are salts of the formula M⁺[BH(alkyl)₃]⁻ (often M⁺[BH(C₁-C₄-alkyl)₃]-), where M⁺ is an alkali metal cation. Examples are Li[BH(ethyl)₃], Na[BH(ethyl)₃], K[BH(ethyl)₃], Li[BH(n-propyl)₃], Na[BH(n-propyl)₃], K[BH(n-propyl)₃], Li[BH(isopropyl)₃], Na[BH(n-propyl)₃], K[BH(n-propyl)₃], Li[BH(n-butyl)₃], Na[BH(n-butyl)₃], K[BH(n-butyl)₃], Li[BH(2-butyl)₃], Na[BH(2-butyl)₃](N-selectride), K[BH(2-butyl)₃](K-selectride) and the like. Other suitable alkali metal borohydrides are salts of the formula M⁺[BH₄]⁻, where M⁺ is an alkali metal cation. A suitable example is LiBH₄.

Suitable alkali metal aluminiumhydrides are salts of the formula M⁺[AlH(alkyl)₃]⁻ (often M⁺[AlH(C₁-C₄-alkyl)₃]⁻), where M⁺ is an alkali metal cation.

Preferably, the alkali metal-containing base is selected from the group consisting of alkali metal C₁-C₁₀-alkoxides, alkali metal amides of the formula M⁺[N(R^{g})₂]⁻, where M⁺ is an alkali metal cation and R^{g} is hydrogen, C₁-C₄-alkyl or Si(C₁-C₄-alkyl)₂; and alkali metal borohydrides of the formula M⁺[BH(C₁-C₄-alkyl)₃]⁻, where M⁺ is an alkali metal cation.

The alkali metal-containing base is more preferably selected from the group consisting of
Na, K or Cs C₁-C₁₀-alkoxides, such as NaOMe, KOMe, NaOEt, KOEt, NaOPr, KOPr, NaOiPr, KOiPr, NaOtBu, KOtBu or CsO(2-ethylhexyl) (preference being given to Na, K or Cs C₄-C₁₀-alkoxides, such as NaOtBu, KOtBu or CsO(2-ethylhexyl));
Li, Na or K amides M⁺[N(R^{g})₂]⁻, where M⁺ is Li⁺, Na⁺ or K⁺ and R^{g} is hydrogen, C₁-C₄-alkyl or Si(C₁-C₄-alkyl)₂, such as LiNH₂, NaNH₂, KNH₂, LiNEt₂ (Et=ethyl), NaNEt₂, KNEt₂, NaHMDS or KHMDS; and
Na or K borohydrides M⁺[BH(C₁-C₄-alkyl)₃]⁻, where M⁺ is Na⁺ or K⁺, such as N-selectride or K-selectride.

Me is methyl, Et is ethyl, Pr is n-propyl, iPr is isopropyl and tBu is tert-butyl.

Specifically, the alkali metal-containing base is selected from the group consisting of KOtBu, CsO(2-ethylhexyl), LiNH₂, LiNEt₂ (Et=ethyl), NaHMDS, KHMDS, N-selectride and K-selectride.

Mixtures of two or more bases can also be used.

The alkali metal-containing base is preferably used in an amount of from 0.00001 to 0.1 mol, more preferably from 0.0001 to 0.05 mol, even more preferably from 0.001 to 0.04 mol, and particularly preferably from 0.01 to 0.04 mol per mol of that compound (II) or (III) which is not used in excess. Alternatively expressed, the alkali metal-containing base is preferably used in an amount of from 0.001 to 10 mol-%, more preferably from 0.01 to 5 mol-%, even more preferably from 0.1 to 4 mol-%, and particularly preferably from 1 to 4 mol-%, relative to the amount (in mol; this amount corresponds to 100 mol-%) of that compound (II) or (III) which is not used in excess.

If compounds (II) or (III) are used in equimolar amounts, the above-given amounts of alkali metal-containing base relate of course to either one of compounds (II) or (III).

In case of compounds (II-2), “equimolar amounts” and “excess” amounts take in this specific context account of the two carboxylate groups (carboxylate groups meaning in this context carboxylic ester groups) which can react in the amidation reaction. Thus, “equimolar” amounts of (II-2) and (III) means in this case 0.5 mol of (II-2) per 1 mol of (III); (II-2) used “in excess” means in this case >0.5 mol of (II-2) per 1 mol of (III), and (III) used “in excess” means in this case >2 mol of (III) per 1 mol of (II-2).

Preferably, the Lewis acid is selected from the group consisting of the halides, carboxylates of the formula R—COO⁻, where R is C₁-C₄-alkyl (to be more precise carboxylate salts where the anion has the formula R—COO⁻, or alternatively expressed salts of carboxylic acids of the formula R—C(O)OH, where R is C₁-C₄-alkyl), carboxylates of the formula R—COO⁻, where R is C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₃-C₆-cycloalkyl-C₁-C₁₀-alkyl), acetylacetonates, C₁-C₄-alkoxides or the carbonyl complexes of Mn, Co, Zn and Bi; and the alkali metal-containing base is selected from the group consisting of alkali metal C₁-C₁₀-alkoxides, alkali metal amides of the formula M⁺[N(R^{g})₂]⁻, where M⁺ is an alkali metal cation and R^{g} is hydrogen, C₁-C₄-alkyl or Si(C₁-C₄-alkyl)₂; and alkali metal borohydrides of the formula M⁺[BH(C₁-C₄-alkyl)₃]⁻, where M⁺ is an alkali metal cation.

More preferably, the Lewis acid is selected from the group consisting of the halides, carboxylates of the formula R—COO⁻, where R is C₁-C₄-alkyl (to be more precise carboxylate salts where the anion has the formula R—COO⁻, or alternatively expressed salts of carboxylic acids of the formula R—C(O)OH, where R is C₁-C₄-alkyl), carboxylates of the formula R—COO⁻, where R is C₃-C₆-cycloalkyl-C₁-C₁₀-alkyl (to be more precise carboxylate salts where the anion has the formula R—COO⁻, or alternatively expressed salts of carboxylic acids of the formula R—C(O)OH, where R is C₃-C₆-cycloalkyl-C₁-C₁₀-alkyl), acetylacetonates, and C₁-C₄-alkoxides of Mn, Co, Zn and Bi, and the carbonyl complexes of Mn; and the alkali metal-containing base is selected from the group consisting of alkali metal C₁-C₁₀-alkoxides, alkali metal amides of the formula M⁺[N(R^{g})₂]⁻, where M⁺ is an alkali metal cation and R^{g} is hydrogen, C₁-C₄-alkyl or Si(C₁-C₄-alkyl)₂; and alkali metal borohydrides of the formula M⁺[BH(C₁-C₄-alkyl)₃]⁻, where M⁺ is an alkali metal cation.

Even more preferably, the Lewis acid is selected from the group consisting of MnCl₂, MnBr₂, BiCl₃, COCl₂, ZnCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀, and the alkali metal-containing base is selected from the group consisting of alkali metal C₁-C₁₀-alkoxides, alkali metal amides of the formula M⁺[N(R^{g})₂]⁻, where M⁺ is an alkali metal cation and R^{g} is hydrogen, C₁-C₄-alkyl or Si(C₁-C₄-alkyl)₂; and alkali metal borohydrides of the formula M⁺[BH(C₁-C₄-alkyl)₃]⁻, where M⁺ is an alkali metal cation.

In particular, the Lewis acid is selected from the group consisting of MnCl₂, MnBr₂, BiCl₃, COCl₂, ZnCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀, and the alkali metal-containing base is selected from the group consisting of KOtBu, CsO(2-ethylhexyl), LiNH₂, LiNEt₂ (Et=ethyl), NaHMDS, KHMDS, N-selectride and K-selectride.

Preferably, the Lewis acid is used in an amount of from 0.00001 to 0.1 mol, and the alkali metal-containing base is used in an amount of from 0.00001 to 0.1 mol, in each case per mol of that compound (II) or (III) which is not used in excess.

More preferably, the Lewis acid is used in an amount of from 0.0001 to 0.05 mol, and the alkali metal-containing base is used in an amount of from 0.0001 to 0.05 mol, in each case per mol of that compound (II) or (III) which is not used in excess.

Even more preferably, the Lewis acid is used in an amount of from 0.001 to 0.01 mol, and the alkali metal-containing base is used in an amount of from 0.001 to 0.04 mol, in each case per mol of that compound (II) or (III) which is not used in excess.

In particular, the Lewis acid is used in an amount of from 0.005 to 0.009 mol, and the alkali metal-containing base is used in an amount of from 0.01 to 0.04 mol, in each case per mol of that compound (II) or (III) which is not used in excess.

Preferably,
the Lewis acid is selected from the group consisting of the halides, carboxylates where the anion has the formula R—COO⁻, where R is C₁-C₄-alkyl, carboxylates where the anion has the formula R—COO⁻, where R is C₃-C₆-cycloalkyl-C₁-C₁₀-alkyl, acetylacetonates, C₁-C₄-alkoxides or the carbonyl complexes of Mn, Co, Zn and Bi;
the alkali metal-containing base is selected from the group consisting of alkali metal C₁-C₁₀-alkoxides, alkali metal amides of the formula M⁺[N(R^{g})₂]⁻, where M⁺ is an al-kali metal cation and R^{g} is hydrogen, C₁-C₄-alkyl or Si(C₁-C₄-alkyl)₂; and alkali metal borohydrides of the formula M⁺[BH(C₁-C₄-alkyl)₃]⁻, where M⁺ is an alkali metal cation;
the Lewis acid is used in an amount of from 0.00001 to 0.1 mol per mol of that compound (II) or (III) which is not used in excess, and
the alkali metal-containing base is used in an amount of from 0.00001 to 0.1 mol per mol of that compound (II) or (III) which is not used in excess.

More preferably,
the Lewis acid is selected from the group consisting of the halides, carboxylates where the anion has the formula R—COO⁻, where R is C₁-C₄-alkyl, carboxylates where the anion has the formula R—COO⁻, where R is C₃-C₆-cycloalkyl-C₁-C₁₀-alkyl, acetylacetonates, and C₁-C₄-alkoxides of Mn, Co, Zn and Bi, and the carbonyl complexes of Mn;
the alkali metal-containing base is selected from the group consisting of alkali metal C₁-C₁₀-alkoxides, alkali metal amides of the formula M⁺[N(R^{g})₂]⁻, where M⁺ is an alkali metal cation and R^{g} is hydrogen, C₁-C₄-alkyl or Si(C₁-C₄-alkyl)₂; and alkali metal borohydrides of the formula M⁺[BH(C₁-C₄-alkyl)₃]⁻, where M⁺ is an alkali metal cation;
the Lewis acid is used in an amount of from 0.0001 to 0.05 mol per mol of that compound (II) or (III) which is not used in excess, and
the alkali metal-containing base is used in an amount of from 0.0001 to 0.05 mol per mol of that compound (II) or (III) which is not used in excess.

Even more preferably,
the Lewis acid is selected from the group consisting of MnCl₂, MnBr₂, BiCl₃, COCl₂, ZnCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀,
the alkali metal-containing base is selected from the group consisting of alkali metal C₁-C₀-alkoxides, alkali metal amides of the formula M⁺[N(R^{g})₂⁻, where M⁺ is an alkali metal cation and R⁹ is hydrogen, C₁-C₄-alkyl or Si(C₁-C₄-alkyl)₂; and alkali metal borohydrides of the formula M⁺[BH(C₁-C₄-alkyl)₃]⁻, where M⁺ is an alkali metal cation;
the Lewis acid is used in an amount of from 0.001 to 0.01 mol per mol of that compound (II) or (III) which is not used in excess, and
the alkali metal-containing base is used in an amount of from 0.001 to 0.04 mol per mol of that compound (II) or (III) which is not used in excess.

Specifically,
the Lewis acid is selected from the group consisting of MnCl₂, MnBr₂, BiCl₃, COCl₂, ZnCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀,
the alkali metal-containing base is selected from the group consisting of KOtBu, CsO(2-ethylhexyl), LiNH₂, LiNEt₂ (Et=ethyl), NaHMDS, KHMDS, N-selectride and K-selectride.
the Lewis acid is used in an amount of from 0.005 to 0.009 mol per mol of that compound (II) or (III) which is not used in excess, and
the alkali metal-containing base is used in an amount of from 0.01 to 0.04 mol per mol of that compound (II) or (III) which is not used in excess.

The reaction can be carried out in one or more solvents. Suitable solvents are all those which do not negatively interfere with the amidation reaction and are suitable to disperse or dissolve the reactants.

Suitable solvents are for example hydrocarbons, such as alkanes, e.g. pentane, hexane, heptane or octane, cycloalkanes, such as cyclopentene, cyclohexane, methylcyclohexane, cycloheptane or cyclooctane, or aromatic compounds, such as benzene, toluene, the xylenes, chlorobenzene, dichlorobenzene, trifluoromethylbenzene or anisole; open-chained ethers, such as diethyl ether, dipropyl ether, dibutyl ether or methyltert-butyl ether, cyclic ethers, such as tetrahydrofuran, 2-methyltetrahydrofuran or 1,4-dioxane, glycol ethers, such as diethyleneglycol dimethyl ether, triethyleneglycol dimethyl ether, polyethyleneglycol dimethyl ether or polypropylenegylcol dimethyl ether, ketones, such as acetone or ethylmethyl ketone, nitriles, such as acetonitrile, sulfoxides, such as dimethylsulfoxides, sulfones, such as sulfolane, alkanols, such as isopropanol or tert-butanol, or mixtures of two or more of the afore-mentioned solvents.

Preference is given to hydrocarbons, in particular to cycloalkanes and aromatic solvents. Specifically, cyclohexane, methylcyclohexane or toluene is used.

Some of the Lewis acids and bases are provided commercially in solvents, e.g. in alkanes, aromatics and/or ethers. In this case, the solvent is often a solvent mixture comprising in addition to the intended solvent also the solvent in which said Lewis acids and bases are provided commercially.

Alternatively, the reaction can be carried out neat, i.e. without any additional solvent, especially if one of the starting materials (II) and/or (III) is liquid under the reaction conditions.

According to the invention, the reaction is carried out under anhydrous conditions, i.e. the water content in the reaction mixture is at most 0.15% by weight, relative to the total weight of the reaction mixture. Preferably, the water content in the reaction mixture is at most 0.1% by weight, more preferably less than 0.1% by weight, and even more preferably less than 0.08% by weight, relative to the total weight of the reaction mixture.

The water content can be calculated from the water content of the starting materials (the reaction has of course to be carried out thus that no or essentially no further water enters the system), or can be determined analytically, e.g. by Karl-Fischer-titration.

The given water content in the reaction mixture generally includes water contained as crystal water. Thus, the reaction being carried out under anhydrous conditions means that the water content in the reaction mixture, inclusive water contained as crystal water, is at most 0.15% by weight, preferably at most 0.1% by weight, more preferably less than 0.1% by weight, even more preferably less than 0.08% by weight, relative to the total weight of the reaction mixture. The reaction mixture is generally composed of the starting materials (II) and (III), the Lewis acid(s), the base(s) and optionally one or more solvents. As the reaction proceeds, the reaction mixture of course also contains the products formed and any intermediates or by-products, as the case may be

Anhydrous conditions can be assured by usual means. For instance, the reactants used (compounds (II) and (III), the Lewis acid(s), the base(s) and optionally the one or more solvents) are provided in anhydrous form, and the reaction is carried out so as to avoid any ingress of humidity, e.g. by using dry apparatuses, by carrying out the reaction in an inert atmosphere, e.g. under (dry) nitrogen or argon, and/or by carrying out the reaction under inherent pressure formed when a closed reaction vessel is used and the reaction is heated (see below remarks), so as to prevent ingress of air (which may bring in humidity).

The reaction is carried out at a temperature of preferably from 80 to 180° C., more preferably from 90 to 160° C., e.g. from 100 to 160° C. or from 120 to 160° C.

The reaction pressure is principally not critical. As however elevated temperatures are preferred and in case that the solvents used have a boiling point beneath the desired temperature, the reaction is in this case generally carried out in a closed vessel or under reflux. This results in an inherent pressure, which is generally in the range of from 1.1 to 30 bar, in particular from 1.5 to 5 bar, specifically from 2 to 4 bar. In another embodiment, when the formed alcohol R⁴OH is higher boiling and should be removed continuously (see below remarks), the pressure can also be reduced in a range of 0.01 to 1 bar, in particular from 0.1 to 1 bar. The reaction pressure can thus range from vacuum over atmospheric pressure to a higher pressure, for example from 0.01 to 30 bar or to 20 bar or to 5 bar or to 4 bar.

If the reaction is carried out under pressure, a closed apparatus is generally used (e.g. an autoclave or other reaction vessels suitable for pressurized reactions), and pressure is either exerted by an inert gas or by heating the reaction mixture, thus inherently causing pressure, or both.

Whether or not the reaction is carried out under pressure, the reaction is preferably carried out under an inert atmosphere, e.g. under (dry) nitrogen or argon.

The reaction is generally carried out by mixing all reactants (starting compounds (II) and (III), Lewis acid(s), base(s)) and optionally one or more solvents in a suitable reaction vessel/reactor and bringing the reaction mixture to the desired temperature for the necessary reaction time, if desired under an inert atmosphere and if desired under pressure (e.g. by using a closed reaction vessel/reactor and optionally inserting an inert gas). Alternatively, the reagents can be added gradually, especially in the case of a continuous or semi-continuous process.

If necessary, the reactants and solvents are dried by usual means before being introduced into the reaction. Solvents may additionally also be degassed.

The reaction can be carried out in apparatuses/reactors customary for the present purpose. It is in principle possible to use any reactor which is suitable for liquid reactions at the desired reaction temperatures and pressures. Suitable standard reactors for gas-liquid and for liquid-liquid reaction systems are known to those skilled in the art and are described, for example, in Ullmann's Encyclopedia of Industrial Chemistry, 2005, Wiley-VCH Verlag GmbH & Co. KGaA, chapter 3.3, Reactor Types and Their Industrial Applications and Reactors for Gas-Liquid Reactions. Suitable examples include, e.g., stirred tank reactors, tubular reactors or bubble column reactors. The reaction may be carried out discontinuously in batch mode or continuously or semi-continuously with recycle or without recycle.

The average reaction time/residence time in the reaction space may be varied in a wide range, preferably in the range from 15 minutes to 100 h, more preferably in the range from 1 to 50 h, e.g. from 1 h to 20 h or from 10 to 20 h.

If desired, the alcohol R⁴—OH formed during amidation can be removed continually or periodically to further the amidation reaction and achieve higher conversion rates. This can for example be achieved by distilling off continuously or periodically the formed alcohol R⁴OH, especially if the reaction is carried out neat or if a solvent with a higher boiling point than the formed alcohol R⁴OH is used, or, in case a solvent with a lower boiling than R⁴OH or a solvent which forms an azeotrope with R⁴OH is used, distilling off R⁴OH together with the organic solvent.

After completion of the reaction to the desired degree, the reaction mixture is worked up by usual means, such as addition of water or an aqueous solution to remove salts (Lewis acids, bases) and phase separation, if necessary/desired neutralization; filtration, extraction, removal of the solvent etc. The suitable work-up depends on the starting materials, solvents used and products formed and can be determined by the skilled person.

The product can be isolated and purified by known means, such as precipitation, filtration, crystallisation, removal of the solvent etc., suitable methods depending on the starting materials, solvents used and products formed, and can be determined by the skilled person.

The present method allows the direct amidation of esters with amines starting from a very broad substrate scope of both esters and amines. In particular, it is possible to use (hetero)aromatic esters and amines, even weakly nucleophilic amines, e.g. such which carry on their (hetero)aromatic moiety electron-withdrawing groups. The Lewis acid and base can be used in substoichiometric, catalytic amounts, thus reducing the amount of potentially hazardous or environmentally problematic and in any case uneconomic waste.

The invention is now illustrated by the following examples.

### Examples

### Analytical Methods

Analytical thin layer chromatography (TLC) was performed on pre-coated Macherey-Nagel ALUGRAM® SIL G/UV254 aluminium sheets.

Standard flash chromatography was performed on an Isolera™ Spektra Systems automated with high performance flash purification system using BIOTAGE® Cartridge Sfär Silica D10, using cyclohexane and ethyl acetate (EtOAc) as eluents. ¹H, ¹³C, and ¹⁹F NMR spectra were recorded in CDCl₃ or d₆-DMSO, on a Bruker AVANCE Ill 300 spectrometer. Chemical shifts are reported in parts per million (ppm) and are referenced to the residual solvent resonance as the internal standard (CHCl₃: δ=7.26 ppm for ¹H NMR and CDCl₃: δ=77.16 ppm for ¹³C NMR). Data are reported as follows: chemical shift, multiplicity (br s=broad singlet, s=singlet, d=doublet, dd=doublet of doublets, dt=doublet of triplets, t=triplet, m=multiplet), coupling constants (Hz), and integration.

Gas liquid chromatography (GLC) was performed on an Agilent Technologies 6890N gas chromatograph equipped with a DB-5 capillary column (30 m×0.32 mm, 0.25 μm film thickness) by CS-Chromatographie Service using the following program: He carrier gas, injection temperature 250° C., detector temperature 300° C., flow rate: 3.42 mL/min; temperature program: start temperature 60° C. for 1 min, heating rate 5° C./min, end temperature 120° C., then heating rate 15° C./min until 270° C., end temperature 270° C. for 2 min. Retention time of mesitylene is 4.65 min. Retention time of p-toluidine (4-methylaniline) is 7.00 min. The retention time of methyl benzoate is 7.56 min. The retention time of N-(p-tolyl)benzamide is 21.75 min.

Abbreviations

| | |
|---|---|
| KOtBu | potassium tert-butanolate |
| EtOAc | ethyl acetate |
| DCM | dichloromethane |
| OAc | acetate |
| OiPr | isopropoxide (isopropanolate) |
| AcAc | acetylacetonate |
| Me | methyl |
| Et | ethyl |
| Ph | phenyl |

### Examples 1 to 11: Preparation of N-(p-tolyl)benzamide—Variation of Lewis Acids

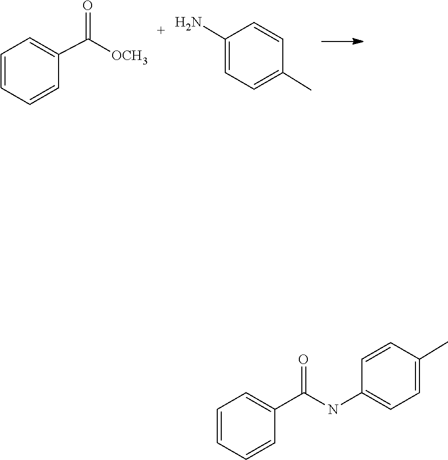

In a glovebox, the Lewis acid indicated in Table 1 below (xx mg; see table 1; in each case 0.01 mmol, 0.84 mol %) followed by KOtBu (4.5 mg, 0.04 mmol, 3.4 mol %), 4-methylaniline (128.6 mg, 1.2 mmol, 1 equiv.; purity >99.9%) and methylbenzoate (150 μL, 1.2 mmol, 1 equiv.; purity >99.9%) were charged into a 38 mL Ace-tube equipped with a magnetic stir bar. Dry and degassed methylcyclohexane (0.3 mL; max. 0.001% water content) was added. The water content in the reaction mixtures (composed of amine, ester, Lewis acid, base, solvent) was maximally 0.1% by weight, relative to the total weight of the reaction mixture (calculated from the given maximum water content of the reagents and solvent used). The tube was sealed with a stopper, removed from the glovebox and inserted into a metal block preheated at 140° C. The reaction was run for 16 h at this temperature while stirred at ca. 750 rpm. The reaction was allowed to cool down to room temperature, diluted with EtOAc or DCM, and mesitylene (138.1 μL, 1.0 mmol, 0.83 equiv.) was added as internal standard. A small aliquot was filtered over a plug of Celite (eluent EtOAc) and analyzed by GC.

In comparative example Comp-1, the reaction was carried out as described above, however without the addition of a Lewis acid.

In comparative examples Comp-2 and Comp-3, the reaction was carried out as described above, however without the addition of a base.

The results are listed in Table 1.

### Examples 12 and 13: Preparation of N-(p-tolyl)benzamide—Variation of Lewis Acids

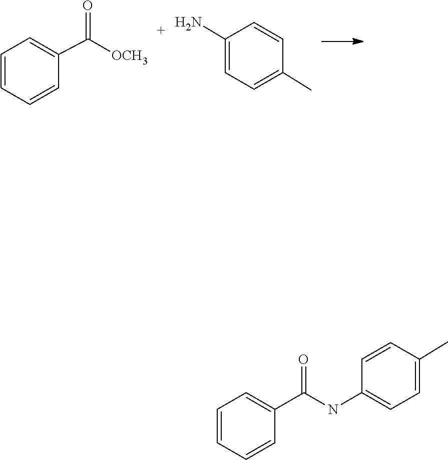

The procedure of examples 1 to 11 was repeated, using however the double amounts of reactants (2.4 mmol of 4-methylaniline and methylbenzoate, respectively, 0.02 mmol of Lewis acid, 0.08 mmol of KOtBu and 0.6 mL of methylcyclohexane)

In comparative example Comp-4, the reaction was carried out as described above, using however a Lewis acid with crystal water. The water content of the reaction mixture was ca. 0.24% by weight, relative to the total weight of the reaction mixture.

The results are listed in Table 1.

TABLE 1

| | | amount | | | GC yield |
|---|---|---|---|---|---|
| | Lewis Acid | LA | | Water | amide |
| Ex. No. | (LA) | [mg] | Base | content* | [%]^{a} |
| 1 | MnCl₂ | 1.3 | KOtBu | ≤0.1% | 95 |
| 2 | MnBr(CO)₅ | 2.7 | KOtBu | ≤0.1% | 79 |
| 3 | Mn(OAc)₂ | 1.7 | KOtBu | ≤0.1% | 72 |
| 4 | Mn(AcAc)₂ | 2.5 | KOtBu | ≤0.1% | 67 |
| 5 | Mn₂(CO)₁₀ | 2.1 | KOtBu | ≤0.1% | 91 |
| 6 | Mn(AcAc)₃ | 3.5 | KOtBu | ≤0.1% | 85 |
| 7 | Mn(4-cyclo- | 4.0 | KOtBu | ≤0.1% | 69 |
| | hexyl- | | | | |
| | butyrate)₂ | | | | |
| 8 | MnBr₂ | 2.2 | KOtBu | ≤0.1% | 93 |
| 9 | ZnCl₂ | 1.4 | KOtBu | ≤0.1% | 66 |
| 10 | BiCl₃ | 3.2 | KOtBu | ≤0.1% | 81 |
| 11 | Bi(OiPr)₃ | 3.9 | KOtBu | ≤0.1% | 73 |
| Comp-1 | — | — | KOtBu | ≤0.1% | 13 |
| Comp-2 | BiCl₃ | 3.2 | — | ≤0.1% | 0 |
| Comp-3 | Bi(OiPr)₃ | 3.9 | — | ≤0.1% | 0 |
| 12^{b} | CoCl₂ | 2.6 | KOtBu | ≤0.1% | 89 |
| 13^{b} | MnCl₂ | 2.5 | KOtBu | ≤0.1% | 95 |
| Comp-4^{b} | MnCl₂*4H₂O | 4.0 | KOtBu | 0.24% | 3 |

^{a}amide: N-(p-tolyl)benzamide. Yield determined with mesitylene as internal standard.
^{b}2.4 mmol scale.
*% by weight, relative to the total weight of the reaction mixture

As Comp-1 shows, the presence of a Lewis acid is necessary for obtaining satisfactory yields.

As Comp-2 and Comp-3 show, the presence of a base is necessary for obtaining satisfactory yields.

Comp-4 shows the importance of anhydrous conditions.

### Examples 14 to 20: Preparation of N-(p-tolyl)benzamide—Variation of Bases

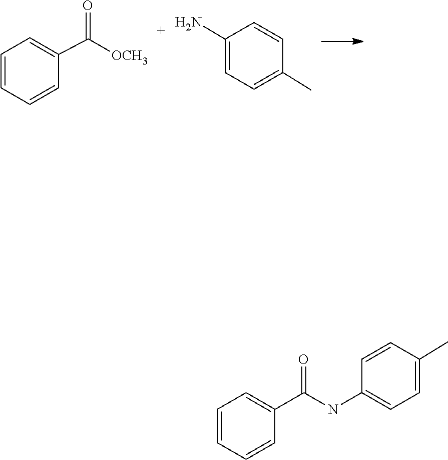

In a glovebox, MnCl₂ (2.5 mg, 0.02 mmol, 0.84 mol %) followed by the base indicated in Table 2 below (xx mg; see table 2; in each case 0.08 mmol, 3.4 mol %), 4-methylaniline (257.2 mg, 2.4 mmol, 1 equiv.) and methylbenzoate (300 μL, 2.4 mmol, 1 equiv.) were charged into a 38 mL Ace-tube equipped with a magnetic stir bar. Dry and degassed methylcyclohexane (0.6 mL; max. 0.001% water content) was added. The water content in the reaction mixtures (composed of amine, ester, Lewis acid, base, solvent) was maximally 0.15% by weight, relative to the total weight of the reaction mixture. The tube was sealed with a stopper, removed from the glovebox and inserted into a metal block preheated at 140° C. The reaction was run for 16 h at this temperature while stirred at ca. 750 rpm. The reaction was allowed to cool down to room temperature, diluted with EtOAc or DCM, and mesitylene (276.2 μL, 2.0 mmol, 0.83 equiv.) was added as an internal standard. A small aliquot was filtered over a plug of Celite (eluent EtOAc) and analyzed by GC.

In comparative example Comp-5, the reaction was carried out as described above, however without the addition of a base.

The results are listed in Table 2.

TABLE 2

| | | | amount | GC yield |
|---|---|---|---|---|
| | | | base | amide |
| Ex. No. | Lewis Acid | Base | [mg] | [%]^{a} |
| 13* | MnCl₂ | KOtBu | 8.9 | 95 |
| 14 | MnCl₂ | KHMDS | 15.9 | 96 |
| 15 | MnCl₂ | NaHMDS | 14.7 | 89 |
| 16 | MnCl₂ | LiNH₂ | 1.8 | 84 |
| 17 | MnCl₂ | Caesium 2- | 80 μL | 98 |
| | | ethylhexoxide^{c} | | |
| 18 | MnCl₂ | N-selectride^{d} | 80 μL | 93 |
| 19 | MnCl₂ | K-selectride^{d} | 80 μL | 89 |
| 20 | MnCl₂ | LiNEt₂^{d} | 6.4 | 92 |
| Comp-5 | MnCl₂ | — | — | 0 |
| Comp-2^{b}* | BiCl₃ | — | — | 0 |
| Comp-3^{b}* | Bi(OiPr)₃ | — | — | 0 |

^{a}amide: N-(p-tolyl)benzamide. Yield determined with mesitylene as internal standard.
^{b}1.2 mmol scale.
^{c}0.8-1.0M in octane/toluene
^{d}1M in THF
*results already depicted in Table 1; included also here for comparison purposes

As Comp-5 as well as Comp-2 and Comp-3 show, the presence of a base is necessary for obtaining satisfactory yields.

### Examples 21 and 22: Preparation of N-(p-tolyl)benzamide—Variation of Leaving Group OR⁴ in Ester

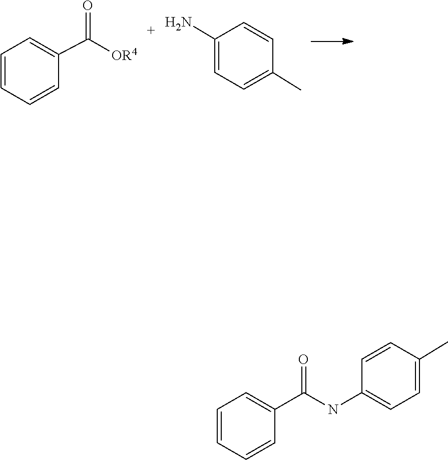

In a glovebox, MnCl₂ (2.5 mg, 0.02 mmol, 0.84 mol %) followed by KOtBu (8.9 mg, 0.08 mmol, 3.4 mol %), 4-methylaniline (257.2 mg, 2.4 mmol, 1 equiv.) and the benzoic acid ester indicated in Table 3 below (2.4 mmol, 1 equiv.) were charged into a 38 mL Ace-tube equipped with a magnetic stir bar. Dry and degassed methylcyclohexane (0.6 mL) was added. The tube was sealed with a stopper, removed from the glovebox and inserted into a metal block preheated at 140° C. The reaction was run for 16 h at this temperature while stirred at ca. 750 rpm. The reaction was allowed to cool down to room temperature, diluted with EtOAc or DCM, and mesitylene (276.2 μL, 2.0 mmol, 0.83 equiv.) was added as an internal standard. A small aliquot was filtered over a plug of Celite (eluent EtOAc) and analyzed by GC.

In comparative example Comp-6, the reaction was carried out as described above, using however the corresponding carboxylic acid instead of an ester.

The results are listed in Table 3.

TABLE 3

| | | | GC yield |
|---|---|---|---|
| | | | amide |
| Ex. No. | OR⁴ | amount ester [mg] | [%]^{a} |
| 13* | OMe | 326.8 mg (ρ: 1.08 g/mL; 300 μL) | 95 |
| 21 | OEt | 360.4 mg (ρ: 1.04 g/mL; 346 μL) | 88 |
| 22 | OPh | 475.7 | 82 |
| Comp-6 | OH | 293.1 | 26 |

^{a}amide: N-(p-tolyl)benzamide. Yield determined with mesitylene as internal standard.
*results already depicted in Table 1; included also here for comparison purposes

### Examples 23 to 47: Variation of Substrates

### General Procedure 1 (1.2 Mmol Scale, Closed System)

In a glovebox, MnCl₂ (1.3 mg, 0.01 mmol, 0.84 mol %) followed by KOtBu (4.5 mg, 0.04 mmol, 3.4 mol %), the corresponding aniline (1.2 mmol, 1 equiv.) and the corresponding ester (1.2 mmol, 1 equiv.) were charged into a 38 mL Ace-tube equipped with a magnetic stir bar. Dry and degassed methylcyclohexane (0.3 mL) was added. The tube was sealed with a stopper, removed from the glovebox and inserted into a metal block preheated at 140° C. The reaction was run for 16 h at this temperature while stirred at ca. 750 rpm. The reaction was allowed to cool down to room temperature, 2 drops H₂O were added, and the mixture was diluted with EtOAc or DCM, stirred for 5 min and filtered over a plug of Celite (eluent EtOAc or DCM). The solvent was removed under reduced pressure and the crude was purified by flash column chromatography on silica gel or by washing with cyclohexane and further drying under hv.

### General Procedure 2 (2.4 Mmol Scale, Closed System)

In a glovebox, MnCl₂ (2.5 mg, 0.02 mmol, 0.84 mol %) followed by KOtBu (8.9 mg, 0.08 mmol, 3.4 mol %), the corresponding aniline (2.4 mmol, 1 equiv.) and the corresponding ester (2.4 mmol, 1 equiv.) were charged into a 38 mL Ace-tube equipped with a magnetic stir bar. Dry and degassed methylcyclohexane (0.6 mL) was added. The tube was sealed with a stopper, removed from the glovebox and inserted into a metal block preheated at 140° C. The reaction was run for 16 h at this temperature while stirred at ca. 750 rpm. The reaction was allowed to cool down to room temperature, 2 drops H₂O were added, and the mixture was diluted with EtOAc or DCM, stirred for 5 min and filtered over a plug of Celite (eluent EtOAc or DCM). The solvent was removed under reduced pressure and the crude was purified by flash column chromatography on silica gel or by washing with cyclohexane and further drying under hv.

### General Procedure 3 (2.4 Mmol Scale, Closed System)

In a glovebox, MnCl₂ (2.5 mg, 0.02 mmol, 0.84 mol %) followed by KOtBu (8.9 mg, 0.08 mmol, 3.4 mol %), the corresponding aniline (2.4 mmol, 1 equiv.) and the corresponding ester (2.4 mmol, 1 equiv.) were charged into a 38 mL Ace-tube equipped with a magnetic stir bar. Dry and degassed methylcyclohexane (0.6 mL) was added. The tube was sealed with a stopper, removed from the glovebox and inserted into a metal block preheated at 160° C. The reaction was run for 16 h at this temperature while stirred at ca. 750 rpm. The reaction was allowed to cool down to room temperature, 2 drops H₂O were added, and the mixture was diluted with EtOAc or DCM, stirred for 5 min and filtered over a plug of Celite (eluent EtOAc or DCM). The solvent was removed under reduced pressure and the crude was purified by flash column chromatography on silica gel or by washing with cyclohexane and further drying under hv.

### Variation of the Amines

### Example 23—Preparation of N-(p-tolyl)benzamide

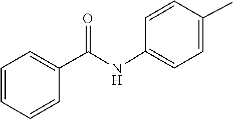 [SMILES: Cc1ccc(NC(=O)c2ccccc2)cc1]

Following General Procedure 1, p-toluidine (128 mg, 1.2 mol, 1 equiv.) and methyl benzoate (150 ul, 1.2 mmol, 1 equiv.) were reacted to afford the amide (215.5 mg, 85%) as a colorless solid after the crude was washed with cyclohexane.

¹H NMR (301 MHz, CDCl₃) δ 8.17 (bs, 1H), 7.90-7.78 (m, 2H), 7.58-7.50 (m, 2H), 7.51-7.46 (m, 1H), 7.40 (dd, J=8.3, 6.8 Hz, 2H), 7.13 (d, J=8.1 Hz, 2H), 2.33 (s, 3H).

¹³C NMR (76 MHz, CDCl₃) δ 166.0, 135.5, 135.1, 134.2, 131.7, 129.6, 128.7, 127.2, 120.6, 21.0.

### Example 24—Preparation of N-(4-methoxyphenyl)benzamide

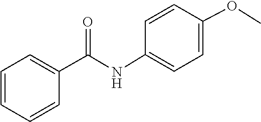 [SMILES: COc1ccc(NC(=O)c2ccccc2)cc1]

Following General Procedure 2, 4-methoxyaniline (295.6 mg, 2.4 mol, 1 equiv.) and methyl benzoate (300 pl, 2.4 mmol, 1 equiv.) were reacted to afford the amide (468.8 mg, 86%) as a colorless solid after the crude was washed with cyclohexane.

¹H NMR (301 MHz, DMSO) δ 10.13 (s, 1H), 7.96 (dt, J=6.6, 1.7 Hz, 2H), 7.76-7.65 (m, 2H), 7.63-7.46 (m, 3H), 7.01-6.86 (m, 2H), 3.75 (s, 3H).

¹³C NMR (76 MHz, DMSO) δ 165.1, 155.6, 135.1, 132.3, 131.4, 128.3, 127.5, 122.0, 113.7, 55.2.

### Example 25—Preparation of N-(4-nitrophenyl)benzamide

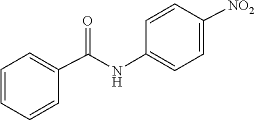 [SMILES: O=C(Nc1ccc([N+](=O)[O-])cc1)c1ccccc1]

Following General Procedure 2, 4-nitroaniline (331.5 mg, 2.4 mol, 1 equiv.) and methyl benzoate (300 pl, 2.4 mmol, 1 equiv.) were reacted to afford the amide (411.9 mg, 71%) as a beige solid after the crude was washed with Et₂O.

¹H NMR (301 MHz, DMSO) δ 10.80 (bs, 1H), 8.30-8.21 (m, 2H), 8.12-8.02 (m, 2H), 8.02-7.92 (m, 2H), 7.69-7.50 (m, 3H).

¹³C NMR (76 MHz, DMSO) δ 166.3, 145.5, 142.4, 134.2, 132.1, 128.5, 127.9, 124.8, 119.8.

### Example 26—Preparation of N-(4-chlorophenyl)benzamide

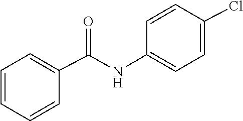 [SMILES: O=C(Nc1ccc(Cl)cc1)c1ccccc1]

Following General Procedure 2, 4-chloroaniline (306.2 mg, 2.4 mol, 1 equiv.) and methyl benzoate (300 pl, 2.4 mmol, 1 equiv.) were reacted to afford the amide (472.4 mg, 85%) as a colorless crystalline powder after the crude was washed with cyclohexane.

¹H NMR (301 MHz, DMSO) δ 10.38 (bs, 1H), 8.02-7.91 (m, 2H), 7.89-7.78 (m, 2H), 7.65-7.47 (m, 3H), 7.45-7.34 (m, 2H).

¹³C NMR (76 MHz, DMSO) δ 165.6, 138.2, 134.7, 131.7, 128.5, 128.4, 127.7, 127.3, 121.8.

### Example 27—Preparation of N-(4-fluorophenyl)benzamide

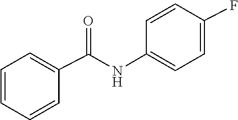 [SMILES: O=C(Nc1ccc(F)cc1)c1ccccc1]

Following General Procedure 2, 4-fluoroaniline (227.9 uL, 1.2 mol, 1 equiv.) and methyl benzoate (300 μl, 12.4 mmol, 1 equiv.) were reacted to afford the amide (423.6 mg, 82%) as a grey solid after the crude was washed with cyclohexane.

### Example 28—Preparation of N-(2-phenylphenyl)benzamide (N-(2-biphenyl)benzamide)

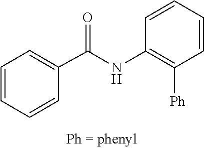 [SMILES: O=C(Nc1ccccc1-c1ccccc1)c1ccccc1]

Following General Procedure 2, 2-aminobiphenyl (406.2 mg, 2.4 mol, 1 equiv.) and methyl benzoate (300 pl, 1.2 mmol, 1 equiv.) were reacted to afford the amide (460.1 mg, 70%) as a colorless solid after the crude was purified by flash column chromatography on silica (0-8% EtOAc in cyclohexane).

¹H NMR (301 MHz, CDCl₃) δ 6.65 (dd, J=8.3, 1.2 Hz, 1H), 6.14 (bs, 1H), 5.76-5.68 (m, 2H), 5.66-5.52 (m, 7H), 5.51-5.45 (m, 2H), 5.42 (dd, J=7.6, 1.7 Hz, 1H), 5.33 (td, J=7.5, 1.2 Hz, 1H).

¹³C NMR (76 MHz, CDCl₃) δ 165.0, 138.2, 135.0, 134.9, 132.5, 131.8, 130.1, 129.4, 129.3, 128.8, 128.7, 128.3, 126.9, 124.5, 121.3.

### Example 29—Preparation of N-(2-fluorophenyl)benzamide

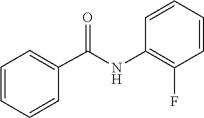 [SMILES: O=C(Nc1ccccc1F)c1ccccc1]

Following General Procedure 2, 2-fluoroaniline (231.9 uL, 2.4 mol, 1 equiv.) and methyl benzoate (300 pl, 2.4 mmol, 1 equiv.) were reacted to afford the amide (411.6 mg, 80%) as a colorless solid after the crude was purified by flash column chromatography on silica (0-10% EtOAc in cyclohexane).

¹H NMR (301 MHz, CDCl₃) δ 8.46 (td, J=8.1, 1.8 Hz, 1H), 8.11 (bs, 1H), 7.96-7.80 (m, 2H), 7.64-7.42 (m, 3H), 7.22-7.02 (m, 3H).

¹⁹F NMR (283 MHz, CDCl₃) δ −131.1.

### Example 30—Preparation of indolin-1-yl(phenyl)methanone

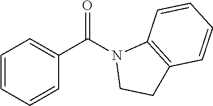 [SMILES: O=C(c1ccccc1)N1CCc2ccccc21]

Following General Procedure 2, indoline (267.3 uL, 2.4 mol, 1 equiv.) and methyl benzoate (300 pl, 2.4 mmol, 1 equiv.) were reacted to afford the amide (434.0 mg, 81%) as colorless solid after the crude was washed with cyclohexane.

### Example 31—Preparation of N-(2-pyridyl)benzamide

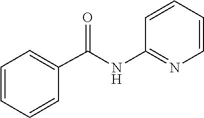 [SMILES: O=C(Nc1ccccn1)c1ccccc1]

Following General Procedure 2, 2-aminopyridine (201.9 mg, 2.4 mol, 1 equiv.) and methyl benzoate (300 pl, 1.2 mmol, 1 equiv.) were reacted to afford the amide (291.0 mg, 61%) as colorless solid after the crude was purified by flash column chromatography (0-40% EtOAc in cyclohexane).

¹H NMR (301 MHz, CDCl₃) δ 9.18 (bs, 1H), 8.46-8.34 (m, 1H), 8.12 (ddd, J=4.9, 1.9, 0.9 Hz, 1H), 7.96-7.87 (m, 2H), 7.73 (ddd, J=8.4, 7.3, 1.9 Hz, 1H), 7.58-7.50 (m, 1H), 7.50-7.42 (m, 2H), 7.01 (ddd, J=7.3, 4.9, 1.0 Hz, 1H).

¹³C NMR (76 MHz, CDCl₃) δ 166.1, 151.9, 147.9, 138.6, 134.5, 132.3, 128.9, 127.4, 119.9, 114.4.

### Example 32—Preparation of N-(8-quinolyl)benzamide

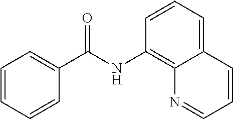 [SMILES: O=C(Nc1cccc2cccnc12)c1ccccc1]

Following General Procedure 2, 8-aminoquinoline (346.0 mg, 2.4 mol, 1 equiv.) and methyl benzoate (300 pl, 1.2 mmol, 1 equiv.) were reacted to afford the amide (359.7 mg, 60%) as colorless solid after the crude was purified by flash column chromatography (0-10% EtOAc in cyclohexane).

¹H NMR (301 MHz, CDCl₃) δ 10.73 (bs, 1H), 8.95 (dd, J=7.5, 1.6 Hz, 1H), 8.82 (dd, J=4.2, 1.7 Hz, 1H), 8.14 (dd, J=8.3, 1.7 Hz, 1H), 8.12-8.06 (m, 2H), 7.62-7.49 (m, 5H), 7.44 (dd, J=8.3, 4.3 Hz, 1H).

¹³C NMR (76 MHz, CDCl₃) δ 165.5, 148.3, 138.8, 136.4, 135.2, 134.7, 131.9, 128.9, 128.1, 127.5, 127.4, 121.8, 121.8, 116.6.

### Example 33—Preparation of N-methyl-N-phenylbenzamide

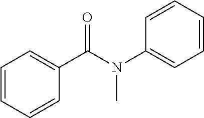 [SMILES: CN(C(=O)c1ccccc1)c1ccccc1]

Following General Procedure 3, N-methylaniline (260.0 μL, 2.4 mol, 1 equiv.) and methyl benzoate (300 pl, 1.2 mmol, 1 equiv.) were reacted to afford the amide (408.5 mg, 80%) as a yellow oil after the crude was purified by flash column chromatography (0-12% EtOAc in cyclohexane).

¹H NMR (301 MHz, CDCl₃) δ 7.35-7.12 (m, 8H), 7.09-7.02 (m, 2H), 3.52 (s, 3H).

### Variation of the Esters

### Example 34—Preparation of 4-fluoro-N-(p-tolyl)benzamide

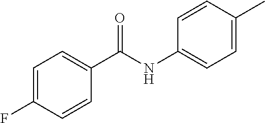 [SMILES: Cc1ccc(NC(=O)c2ccc(F)cc2)cc1]

Following General Procedure 2, p-toluidine (257.2 mg, 2.4 mol, 1 equiv.) and methyl 4-fluorobenzoate (311 pl, 2.4 mmol, 1 equiv.) were reacted to afford the amide (445.5 mg, 81%) as a colorless powder after the crude was washed with cyclohexane.

¹H NMR (301 MHz, CDCl₃) δ 7.91-7.83 (m, 2H), 7.82 (d, J=7.4 Hz, 1H), 7.53-7.45 (m, 2H), 7.20-7.08 (m, 4H), 2.34 (s, 3H).

¹⁹F NMR (283 MHz, CDCl₃) δ −107.68.

¹³C NMR (76 MHz, CDCl₃) δ 166.7, 164.1 (d, J=112.1 Hz), 135.2, 134.4, 129.6, 129.4 (d, J=9.1 Hz),120.4, 115.8 (d, J=22.0 Hz), 20.9.

### Example 35—Preparation of 4-methoxy-N-(p-tolyl)benzamide

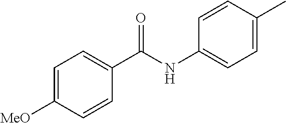 [SMILES: COc1ccc(C(=O)Nc2ccc(C)cc2)cc1]

Following General Procedure 2, p-toluidine (257.2 mg, 2.4 mol, 1 equiv.) and methyl 4-methoxybenzoate (398.3 mg, 2.4 mmol, 1 equiv.) were reacted to afford the amide (495.4 mg, 86%) as a colorless white powder after the crude was washed with cyclohexane.

¹H NMR (301 MHz, CDCl₃) δ 7.89-7.79 (m, 3H), 7.55-7.47 (m, 2H), 7.18-7.11 (m, 2H), 6.98-6.90 (m, 2H), 3.85 (s, 3H), 2.33 (s, 3H).

¹³C NMR (76 MHz, CDCl₃) δ 165.4, 162.5, 135.7, 134.1, 129.6, 129.0, 127.4, 120.5, 114.20, 55.6, 21.0.

### Example 36—Preparation of 3-methyl-N-(p-tolyl)benzamide

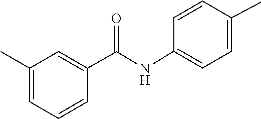

Following General Procedure 2, p-toluidine (257.2 mg, 2.4 mol, 1 equiv.) and methyl m-toluate (344.2 μL, 2.4 mmol, 1 equiv.) were reacted to afford the amide (393.0 mg, 73%) as a colorless solid after the crude was purified by flash column chromatography (0-15% EtOAc in cyclohexane).

¹H NMR (301 MHz, CDCl₃) δ 7.86 (bs, 1H), 7.67 (q, J=1.2 Hz, 1H), 7.63 (ddd, J=5.3, 3.6, 2.0 Hz, 1H), 7.55-7.49 (m, 2H), 7.36-7.30 (m, 2H), 7.19-7.13 (m, 2H), 2.41 (s, 3H), 2.34 (s, 3H).

¹³C NMR (76 MHz, CDCl₃) δ 166.0, 138.7, 135.6, 135.2, 134.2, 132.6, 129.7, 128.7, 127.9, 124.1, 120.4, 21.5, 21.0.

### Example 37—Preparation of 3-fluoro-N-(p-tolyl)benzamide

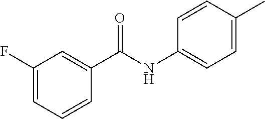 [SMILES: Cc1ccc(NC(=O)c2cccc(F)c2)cc1]

Following General Procedure 2, p-toluidine (257.2 mg, 2.4 mol, 1 equiv.) and methyl 3-fluorobenzoate (315.9 μL, 2.4 mmol, 1 equiv.) were reacted to afford the amide (339.9 mg, 62%) as a colorless solid after the crude was purified by flash column chromatography (0-10% EtOAc in cyclohexane).

¹H NMR (301 MHz, CDCl₃) δ 7.97 (bs, 1H), 7.63-7.58 (m, 1H), 7.55 (ddd, J=9.3, 2.6, 1.6 Hz, 1H), 7.52-7.46 (m, 2H), 7.40 (td, J=8.0, 5.5 Hz, 1H), 7.21 (tdd, J=8.3, 2.6, 1.0 Hz, 1H), 7.14 (d, J=8.3 Hz, 2H), 2.33 (s, 3H).

¹⁹F NMR (283 MHz, CDCl₃) δ −111.45.

### Example 38—Preparation of N-(p-tolyl)pyridine-3-carboxamide

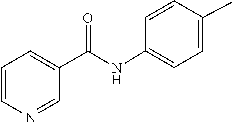 [SMILES: Cc1ccc(NC(=O)c2cccnc2)cc1]

Following General Procedure 2, p-toluidine (257.2 mg, 2.4 mol, 1 equiv.) and methyl nicotinate (328.9 mg, 2.4 mmol, 1 equiv.) were reacted to afford the amide (402.5 mg, 79%) as a brown solid after the crude was washed with cyclohexane.

¹H NMR (301 MHz, DMSO) δ 10.38 (s, 1H), 9.11 (d, J=2.5 Hz, 1H), 8.75 (dd, J=4.8, 1.7 Hz, 1H), 8.29 (dt, J=8.0, 2.1 Hz, 1H), 7.66 (d, J=8.5 Hz, 2H), 7.55 (dd, J=8.0, 4.7 Hz, 1H), 7.17 (d, J=8.4 Hz, 2H), 2.28 (s, 3H).

¹³C NMR (76 MHz, DMSO) δ 163.8, 152.0, 148.7, 136.3, 135.4, 133.0, 130.7, 129.1, 123.5, 120.4, 20.5.

### Example 39—Preparation of N-(p-tolyl)pyridine-2-carboxamide

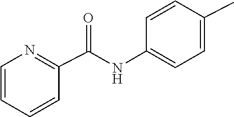 [SMILES: Cc1ccc(NC(=O)c2ccccn2)cc1]

Following General Procedure 2, p-toluidine (257.2 mg, 2.4 mol, 1 equiv.) and methyl picolinate (289.5 μL, 2.4 mmol, 1 equiv.) were reacted to afford the amide (452.9 mg, 89%) as a light brown solid after the crude was washed with cyclohexane.

¹H NMR (301 MHz, CDCl₃) δ 9.96 (bs, 1H), 8.58 (dd, J=4.9, 1.7 Hz, 1H), 8.33-8.23 (m, 1H), 7.87 (td, J=7.7, 1.6 Hz, 1H), 7.71-7.62 (m, 2H), 7.44 (ddd, J=7.7, 4.7, 1.2 Hz, 1H), 7.18 (d, J=8.2 Hz, 2H), 2.33 (s, 3H).

¹³C NMR (76 MHz, CDCl₃) δ 161.9, 145.0, 148.0, 137.7, 135.3, 133.9, 129.6, 126.4, 122.4, 119.7, 21.0.

### Example 40—Preparation of 2-phenyl-N-(p-tolyl)acetamide

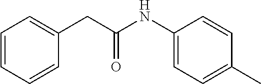 [SMILES: Cc1ccc(NC(=O)Cc2ccccc2)cc1]

Following General Procedure 2, p-toluidine (257.2 mg, 2.4 mol, 1 equiv.) and methyl phenylacetate (364.0 μL, 2.4 mmol, 1 equiv.) were reacted to afford the amide (479.2 mg, 89%) as a colorless solid after the crude was washed with cyclohexane.

¹H NMR (301 MHz, CDCl₃) δ 7.49 (bs, 1H), 7.41-7.27 (m, 7H), 7.07 (d, J=8.2 Hz, 2H), 3.69 (s, 2H), 2.30 (s, 3H).

¹³C NMR (76 MHz, CDCl₃) δ 164.4, 135.3, 134.8, 134.1, 129.6, 129.5, 129.2, 127.6, 120.2, 44.7, 20.9.

### Example 41—Preparation of 3-phenyl-N-(p-tolyl)propanamide

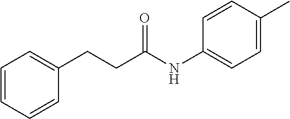 [SMILES: Cc1ccc(NC(=O)CCc2ccccc2)cc1]

Following General Procedure 2, p-toluidine (257.2 mg, 2.4 mol, 1 equiv.) and methyl 3-phenylpropionate (378.9 μL, 2.4 mmol, 1 equiv.) were reacted to afford the amide (464.1 mg, 81%) as a colorless powder after the crude was washed with cyclohexane.

¹H NMR (301 MHz, CDCl₃) δ 7.37-7.24 (m, 5H), 7.27-7.15 (m, 3H), 7.08 (d, J=8.2 Hz, 2H), 3.03 (t, J=7.6 Hz, 2H), 2.67-2.59 (m, 2H), 2.30 (s, 3H).

¹³C NMR (76 MHz, CDCl₃) δ 170.5, 140.8, 135.3, 134.0, 129.5, 128.7, 128.5, 126.4, 120.3, 39.4, 31.72 21.0.

### Example 42—Preparation of (E)-3-phenyl-N-(p-tolyl)prop-2-enamide

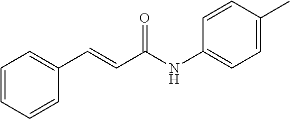 [SMILES: Cc1ccc(NC(=O)/C=C/c2ccccc2)cc1]

Following General Procedure 2, p-toluidine (257.2 mg, 2.4 mol, 1 equiv.) and methyl cinnamate (389.3 mg, 2.4 mmol, 1 equiv.) were reacted to afford the amide (447.5 mg, 72%) as a colorless solid after the crude was purified by flash column chromatography (0-100% EtOAc in cyclohexane).

¹H NMR (301 MHz, CDCl₃) δ 7.75 (d, J=15.5 Hz, 1H), 7.57-7.45 (m, 4H), 7.42-7.35 (m, 3H), 7.33 (bs, 1H), 7.15 (d, J=8.3 Hz, 2H), 6.54 (d, J=15.5 Hz, 1H), 2.33 (s, 3H).

¹³C NMR (76 MHz, CDCl₃) δ 164.5, 142.0, 135.7, 134.8, 134.2, 129.9, 129.6, 128.9, 128.0, 121.3, 120.6, 21.0.

### Example 43—Preparation of N-(p-tolyl)oleoylamide

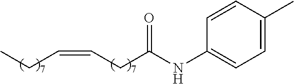

Following General Procedure 2, p-toluidine (257.2 mg, 2.4 mol, 1 equiv.) and methyl oleate (214.2 μL, 2.4 mmol, 1 equiv.) were reacted to afford the amide (755.6 mg, 85%) as a light yellow waxy solid after the crude was purified by flash column chromatography (0-15% EtOAc in cyclohexane).

¹H NMR (301 MHz, CDCl₃) δ 7.44 (bs, 1H), 7.44-7.35 (m, 2H), 7.09 (d, J=8.2 Hz, 2H), 5.42-5.27 (m, 2H), 2.37-2.24 (m, 5H), 2.08-1.93 (m, 4H), 1.78-1.61 (m, 2H), 1.43-1.20 (m, 20H), 0.94-0.83 (m, 3H).

¹³C NMR (76 MHz, CDCl₃) δ 171.6, 135.6, 133.8, 130.1, 129.8, 129.5, 120.1, 37.8, 32.0, 29.9, 29.8, 29.6, 29.4, 29.4, 29.3, 27.3, 27.3, 25.8, 22.8, 20.9, 14.2.

### Example 44—Preparation of 3-(difluoromethyl)-1-methyl-N-(p-tolyl)pyrazole-4-carboxamide

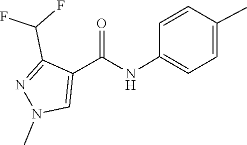 [SMILES: Cc1ccc(NC(=O)c2cn(C)nc2C(F)F)cc1]

Following General Procedure 1, p-toluidine (128 mg, 1.2 mol, 1 equiv.) and 3-difluoromethyl-1-methyl-1H-pyrazole-4-carboxylic acid methyl ester (228.1 mg, 1.2 mmol, 1 equiv.) were reacted to afford the amide (220.5 mg, 69%) as a colorless solid after the crude was purified by flash column chromatography (0-10% EtOAc in cyclohexane).

¹H NMR (301 MHz, CDCl₃) δ 8.53 (bs, 1H), 7.94-7.82 (m, 1H), 7.44-7.35 (m, 2H), 7.17 (t, J=54.1 Hz, 1H), 7.10-7.04 (m, 2H), 3.75 (s, 3H), 2.29 (s, 3H).

¹⁹F NMR (283 MHz, CDCl₃) δ −111.26.

¹³C NMR (76 MHz, CDCl₃) δ 160.0, 144.1 (t, J=26.4 Hz), 135.0, 134.4, 133.9, 129.4, 120.8, 116.7, 111.0 (t, J=234.2 Hz), 39.3, 20.8.

### Variation of Ester and Amine

### Example 45—Preparation of 4-cyano-N-(2-fluorophenyl)benzamide

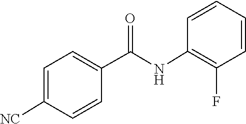 [SMILES: N#Cc1ccc(C(=O)Nc2ccccc2F)cc1]

Following General Procedure 2, 2-fluoroaniline (231.9 uL, 2.4 mol, 1 equiv.) and methyl 4-cyanobenzoate (327.5 μL, 2.4 mmol, 1 equiv.) were reacted for 24 h to afford the amide (508.7 mg, 88%) as a colorless solid after the crude was purified by flash column chromatography (0-20% EtOAc in cyclohexane).

¹H NMR (301 MHz, CDCl₃) δ 8.40 (ddd, J=8.2, 6.9, 1.3 Hz, 1H), 8.06 (s, 1H), 8.02-7.95 (m, 2H), 7.84-7.76 (m, 2H), 7.24-7.09 (m, 3H).

¹⁹F NMR (283 MHz, CDCl₃) δ −130.75.

### Example 46—Preparation of indolin-1-yl-(3,4,5-trimethoxyphenyl)methanone

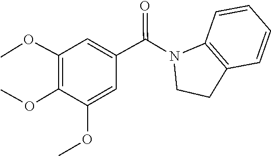 [SMILES: COc1cc(C(=O)N2CCc3ccccc32)cc(OC)c1OC]

Following General Procedure 2, indoline (267.3 μL, 2.4 mol, 1 equiv.) and methyl 3,4,5-trimethoxybenzoate (542.9 mg, 2.4 mmol, 1 equiv.) were reacted to afford the amide (691.8 mg, 92%) as a colorless solid after the crude was purified by flash column chromatography (0-20% EtOAc in cyclohexane).

¹H NMR (301 MHz, CDCl₃) δ 7.20 (d, J=7.3 Hz, 1H), 7.11 (bs, 1H), 7.00 (t, J=7.4 Hz, 1H), 6.77 (s, 2H), 4.19-4.03 (m, 2H), 3.89 (s, 3H), 3.85 (s, 6H), 3.12 (t, J=8.2 Hz, 2H).

### Diamides

### Example 47—Preparation of N,N′-bis(p-tolyl)propanediamide

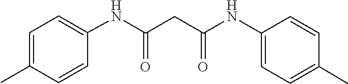

In a glovebox, MnCl₂ (2.5 mg, 0.02 mmol, 0.84 mol %) followed by KOtBu (8.9 mg, 0.08 mmol, 3.4 mol %), p-toluidine (512.0 mg, 4.8 mmol, 2 equiv.) and methyl malonate (273.4 μL, 2.4 mmol, 1 equiv.) were charged into a 38 mL Ace-tube equipped with a magnetic stir bar. Dry and degassed methylcyclohexane (0.6 mL) was added. The tube sealed with a stopper, removed from the glovebox and inserted into a metal block preheated at 140° C. The reaction was run for 16 h at this temperature while stirred at ca. 750 rpm. The reaction was allowed to cool down to room temperature, 2 drops H₂O were added, and the mixture was diluted with EtOAc or DCM, stirred for 5 min and filtered over a plug of Celite. Most of the reaction mixture was not soluble in these solvents. After the plug was eluted with EtOAc, the remaining material was dissolved in acetone and filtered over Celite. The acetone fractions were collected and the solvent was removed under reduced pressure to afford the bisamide (355.0 mg, 52%) as a white powder.

¹H NMR (301 MHz, DMSO) δ 10.06 (bs, 2H), 7.68-7.32 (m, 4H), 7.30-6.92 (m, 4H), 3.43 (s, 2H), 2.25 (s, 6H).

¹³C NMR (76 MHz, DMSO) δ 165.2, 136.5, 132.3, 129.1, 119.1, 45.8, 20.4.

### Example 48—Preparation of N-(p-tolyl)benzamide—Open System

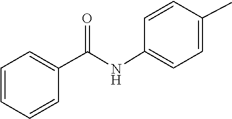 [SMILES: Cc1ccc(NC(=O)c2ccccc2)cc1]

In a glovebox, MnCl₂ (5.2 mg, 0.04 mmol, 0.84 mol %) followed by KOtBu (18.0 mg, 0.16 mmol, 3.4 mol %), 4-methylaniline (514.4 mg, 4.8 mmol, 1 equiv.) and methylbenzoate (600 uL, 4.8 mmol, 1 equiv.) were charged into a 25 mL round-bottom flask equipped with a magnetic stir bar. Dry and degassed toluene (6 mL) was added. The flask was closed with a rubber septum and removed from the glovebox. The flask was connected to a dry reflux condenser under Argon (note: no water in the cooling part). The top of the condenser was closed with a rubber septum and a long needle (0.80×120 mm) was used as an Ar inlet. A shorter needle (0.9×40 mm) was inserted in order to generate an Ar flow. The flask was inserted into a preheated oil bath (oil temperature 140° C.) and refluxed for 17 h under a flow of Ar. (note: no water cooling during this time). The reaction was allowed to cool down to room temperature. Most of the volume of the solvent evaporated during the course of the reaction. The reaction was diluted with EtOAc and filtered over a plug of Celite (eluent EtOAc). The solvent was removed under reduced pressure and the crude was diluted in a minimum amount of DCM. 10 mL of cylohexane were added and DCM evaporated under reduced pressure in order to precipitate the product as a colorless solid. Ca. 50% of the cyclohexane was further removed under reduced pressure before the remainder was removed (Pasteur pipette). The precipitate was washed with cyclohexane and further dried under reduced pressure to afford the title compound (947.3 mg, 93%) as a colorless solid.

¹H NMR (301 MHz, CDCl₃) δ 8.17 (bs, 1H), 7.90-7.78 (m, 2H), 7.58-7.50 (m, 2H), 7.51-7.46 (m, 1H), 7.40 (dd, J=8.3, 6.8 Hz, 2H), 7.13 (d, J=8.1 Hz, 2H), 2.33 (s, 3H).

¹³C NMR (76 MHz, CDCl₃) δ 166.0, 135.5, 135.1, 134.2, 131.7, 129.6, 128.7, 127.2, 120.6, 21.0.

1. A method for preparing an amide of the formula (I-1) or a diamide of the formula (I-2) 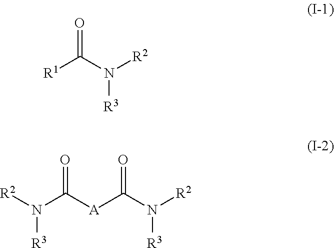
where
R¹ is selected from the group consisting of hydrogen, C₁-C₃₀-alkyl which is unsubstituted or carries m radicals R^{a}, C₁-C₃₀-haloalkyl which is unsubstituted or carries m radicals R^{a}, C₂-C₃₀-alkenyl which is unsubstituted or carries m radicals R^{a}, C₂-C₃₀-haloalkenyl which is unsubstituted or carries m radicals R^{a}, C₂-C₃₀-alkynyl which is unsubstituted or carries m radicals R^{a}, C₂-C₃₀-haloalkynyl which is unsubstituted or carries m radicals R^{a}, C₃-C₃₀-cycloalkyl which is unsubstituted or carries m radicals R^{b}, C₆-C₂₂-aryl which is unsubstituted or carries m radicals R^{b}, and a 3- to 30-membered saturated, partially unsaturated, or maximally unsaturated heterocyclic ring containing 1, 2, 3, or 4 heteroatoms or heteroatom groups selected from, N, O, S, SO, and SO₂ as ring members, which is unsubstituted or carries m radicals R^{b};
R² is C₆-C₂₂-aryl which is unsubstituted or carries m radicals R^{b}, or is a 5- to 30-membered heteroaromatic ring containing 1, 2, 3, or 4 heteroatoms selected from N, O, and S as ring members, where the heteroaromatic ring is unsubstituted or carries m radicals R^{b};
R³ is selected from the group consisting of hydrogen, C₁-C₃₀-alkyl which is unsubstituted or carries m radicals R^{a}, C₁-C₃₀-haloalkyl which is unsubstituted or carries m radicals R^{a}, C₂-C₃₀-alkenyl which is unsubstituted or carries m radicals R^{a}, C₂-C₃₀-haloalkenyl which is unsubstituted or carries m radicals R^{a}, C₂-C₃₀-alkynyl which is unsubstituted or carries m radicals R^{a}, C₂-C₃₀-haloalkynyl which is unsubstituted or carries m radicals R^{a}, C₃-C₃₀-cycloalkyl which is unsubstituted or carries m radicals R^{b}, C₆-C₂₂-aryl which is unsubstituted or carries m radicals R^{b}, and a 3- to 30-membered saturated, partially unsaturated, or maximally unsaturated heterocyclic ring containing 1, 2, 3, or 4 heteroatoms or heteroatom groups selected from, N, O, S, SO, and SO₂ as ring members, which is unsubstituted or carries m radicals R^{b};
or
R³ forms a saturated or unsaturated 2-, 3-, or 4-membered linking group to a carbon or nitrogen ring atom of the aryl or heteroaromatic ring R²; where the linking group may comprise 1 or 2 heteroatoms or heteroatom groups selected from, N, O, S, SO, and SO₂; where the linking group may carry 1, 2, or 3 radicals selected from the group consisting of halogen, cyano, C₁-C₄-alkyl, C₁-C₄-haloalkyl, C₁-C₄-alkoxy, and C₁-C₄-haloalkoxy;
A is a divalent aliphatic, cycloaliphatic, aliphatic-cycloaliphatic aromatic, aromatic-aliphatic, or heterocyclic moiety;
each R^{a} is independently selected from the group consisting of cyano, nitro, hydroxyl, C₁-C₄-alkoxy, C₁-C₄-haloalkoxy, C(═O)R^{c}, amino, C₁-C₄-alkylamino, di-(C₁-C₄-alkyl)-amino, C₃-C₂₀-cycloalkyl, C₆-C₂₂-aryl which is unsubstituted or carries m radicals R^{d}, and a 3- to 20-membered saturated, partially unsaturated, or maximally unsaturated heterocyclic ring containing 1, 2, 3, or 4 heteroatoms or heteroatom groups selected from, N, O, S, SO, and SO₂ as ring members, which is unsubstituted or carries m radicals R^{d};
each R^{b} is independently selected from the group consisting of halogen, cyano, nitro, hydroxyl, C₁-C₄-alkoxy, C₁-C₄-haloalkoxy, amino, C₁-C₄-alkylamino, di-(C₁-C₄-alkyl)-amino, NR^{e}R^{f}, C(═O)NR^{e}R^{f}, C₁-C₂₀-alkyl, C₁-C₂₀-haloalkyl, C₂-C₂₀-alkenyl, C₂-C₂₀-haloalkenyl, C₂-C₂₀-alkynyl, C₂-C₂₀-haloalkynyl, C₃-C₂₀-cycloalkyl, C₆-C₂₂-aryl which is unsubstituted or carries m radicals R^{d}, and a 3- to 20-membered saturated, partially unsaturated, or maximally unsaturated heterocyclic ring containing 1, 2, 3, or 4 heteroatoms or heteroatom groups selected from, N, O, S, SO, and SO₂ as ring members, which is unsubstituted or carries m radicals R^{d};
each R^{c} is independently selected from the group consisting of C₁-C₄-alkyl, C₁-C₄-haloalkyl, C₁-C₄-alkyl which carries a group NR^{e}R^{f}, C₁-C₄-alkoxy, and C₁-C₄-haloalkoxy;
each R^{d} is independently selected from the group consisting of halogen, cyano, hydroxyl, C₁-C₄-alkyl, C₁-C₄-haloalkyl, C₁-C₄-alkoxy, and C₁-C₄-haloalkoxy;
each R^{e} is independently selected from the group consisting of hydrogen and C₁-C₄-alkyl;
each R^{f} is independently selected from the group consisting of —C(═O)-phenyl and phenyl which is unsubstituted or substituted by 1, 2, or 3 radicals selected from the group consisting of halogen, cyano, C₁-C₄-alkyl, C₁-C₄-haloalkyl, C₁-C₄-alkoxy, and C₁-C₄-haloalkoxy;
each m is independently 1, 2, 3, 4, or 5;
comprising reacting an ester compound (II) of formula (II-1) or (II-2) 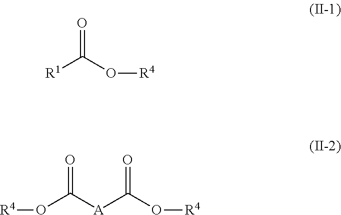
wherein
R¹ and A are as defined above; and
R⁴ is selected from the group consisting of C₁-C₃₀-alkyl, C₆-C₁₄-aryl, and C₆-C₁₄-aryl-C₁-C₄-alkyl;
with an amine of formula (III) 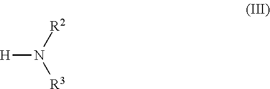
wherein R² and R³ are as defined above,
in the presence of an alkali metal-containing base and a Lewis acid;
where the reaction is carried out under anhydrous conditions, where a water content in the reaction mixture is at most 0.15% by weight, relative to a total weight of the reaction mixture.

2. The method according to claim 1, where 1, 2, 3, or 4 of the following conditions a), b), c), and/or e); or 1, 2, 3, or 4 of the following conditions a), b), d), and/or e) apply:
a) R¹ is selected from the group consisting of C₁-C₂₀-alkyl, C₁-C₄-alkyl which carries 1 or 2 radicals R^{a}, C₂-C₂₀-alkenyl, C₂-C₄-alkenyl which carries a phenyl ring, C₃-C₆-cycloalkyl which is unsubstituted or carries m radicals R^{b}, C₆-C₁₀-aryl which is unsubstituted or carries m radicals R^{b}, and a 5- to 10-membered heteroaromatic ring containing 1, 2, 3, or 4 heteroatoms selected from, N, O, and S as ring members, which is unsubstituted or carries m radicals R^{b};
where
each R^{a} is independently C₁-C₄-alkoxy, C₁-C₄-haloalkoxy, C(═O)R^{c}, or phenyl; and
each R^{b} is independently halogen, cyano, C₁-C₄-alkyl, C₁-C₄-haloalkyl, C₁-C₄-alkoxy, C₁-C₄-haloalkoxy, and a 5- or 6-membered heteroaromatic ring containing 1, 2, 3, or 4 heteroatoms selected from, N, O, and S as ring members, which is unsubstituted or carries m radicals R^{d}; where each R^{d} is independently selected from the group consisting of halogen, cyano, C₁-C₄-alkyl, C₁-C₄-haloalkyl, C₁-C₄-alkoxy, and C₁-C₄-haloalkoxy;
b) R² is selected from the group consisting of C₆-C₁₀-aryl which is unsubstituted or carries m radicals R^{b}, and a 5- to 10-membered heteroaromatic ring containing 1, 2, 3, or 4 heteroatoms selected from N, O, and S as ring members, which is unsubstituted or carries m radicals R^{b};
where
each R^{b} is independently selected from the group consisting of halogen, cyano, hydroxyl, nitro, C(═O)NR^{e}R^{f}, C₁-C₄-alkyl, C₁-C₄-haloalkyl, C₁-C₄-alkoxy, C₁-C₄-haloalkoxy, and phenyl which is unsubstituted or carries m radicals R^{d};
each R^{d} is independently selected from the group consisting of halogen, C₁-C₄-alkyl, and C₁-C₄-haloalkyl;
each R^{e} is independently selected from the group consisting of hydrogen and C₁-C₄-alkyl; and
each R^{f} is independently selected from the group consisting of —C(═O)-phenyl and phenyl which is unsubstituted or substituted by 1, 2, or 3 radicals selected from the group consisting of halogen, cyano, C₁-C₄-alkyl, C₁-C₄-haloalkyl, C₁-C₄-alkoxy, and C₁-C₄-haloalkoxy;
c) R³ is hydrogen or C₁-C₄-alkyl;
d) R³ forms a linking group —(CH₂)₂—, —(CH₂)₃—, or —CH═CH— to a carbon or nitrogen ring atom of the aryl or heteroaromatic ring R²;
e) A is C₁-C₈-alkanediyl, C₂-C₈-alkenediyl, C₃-C₆-cycloalkanediyl, or phenylene.

3. The method according to claim 1, where R¹ is selected from the group consisting of C₁-C₂₀-alkyl, C₁-C₄-alkyl which carries a phenyl ring, C₂-C₂₀-alkenyl, C₂-C₄-alkenyl which carries a phenyl ring, C₆-C₁₀-aryl which is unsubstituted or carries m radicals R^{b}, and a 5- to 10-membered heteroaromatic ring containing 1, 2, 3, or 4 heteroatoms selected from, N, O, and S as ring members, which is unsubstituted or carries m radicals R^{b};
where each R^{b} is independently halogen, cyano, C₁-C₄-alkyl, C₁-C₄-haloalkyl, C₁-C₄-alkoxy, and C₁-C₄-haloalkoxy.

4. The method according to claim 3, where R¹ is selected from the group consisting of C₁-C₄-alkyl which carries a phenyl ring, C₂-C₂₀-alkenyl, C₂-C₄-alkenyl which carries a phenyl ring, phenyl which is unsubstituted or carries m radicals R^{b}, and a 5- or 6-membered heteroaromatic ring containing 1, 2, or 3 heteroatoms selected from, N, O, and S as ring members, which is unsubstituted or carries m radicals R^{b};
where each R^{b} is independently halogen, cyano, C₁-C₄-alkyl, C₁-C₄-haloalkyl, C₁-C₄-alkoxy, and C₁-C₄-haloalkoxy; and m is 1, 2, or 3.

5. The method according to claim 4, where R¹ is selected from the group consisting of C₁-C₂-alkyl which carries a phenyl ring, C₂-C₂₀-alkenyl, C₂-alkenyl which carries a phenyl ring, phenyl which is unsubstituted or carries m radicals R^{b}, and a 5- or 6-membered heteroaromatic ring containing 1 or 2 nitrogen atoms as ring members, which is unsubstituted or carries m radicals R^{b};
where each R^{b} is independently halogen, cyano, C₁-C₄-alkyl, C₁-C₄-haloalkyl, C₁-C₄-alkoxy, and C₁-C₄-haloalkoxy; and m is 1, 2, or 3.

6. The method according to claim 1, where R² is selected from the group consisting of C₆-C₁₀-aryl which is unsubstituted or carries m radicals R^{b}, and a 5- to 10-membered heteroaromatic ring containing 1, 2, 3, or 4 heteroatoms selected from N, O, and S as ring members, which is unsubstituted or carries m radicals R^{b};
where each R^{b} is independently selected from the group consisting halogen, cyano, nitro, C₁-C₄-alkyl, C₁-C₄-haloalkyl, C₁-C₄-alkoxy, C₁-C₄-haloalkoxy, and phenyl; and m is 1, 2, or 3.

7. The method according to claim 6, where R² is selected from the group consisting of phenyl which is unsubstituted or carries m radicals R^{b}, and 6- to 10-membered heteroaromatic ring containing 1 or 2 nitrogen atoms as ring members, which is unsubstituted or carries m radicals R^{b};
where each R^{b} is independently selected from the group consisting halogen, nitro, C₁-C₄-alkyl, C₁-C₄-alkoxy, and phenyl; and m is 1, 2, or 3.

8. The method according to claim 1, where R⁴ is selected from the group consisting of C₁-C₄-alkyl, phenyl, and benzyl.

9. The method according to claim 1, where
R¹ is selected from the group consisting of C₁-C₂-alkyl which carries a phenyl ring, C₂-C₂₀-alkenyl, C₂-alkenyl which carries a phenyl ring, phenyl which is unsubstituted or carries m radicals R^{b}, and a 5- or 6-membered heteroaromatic ring containing 1 or 2 nitrogen atoms as ring members, which is unsubstituted or carries m radicals R^{b};
where
each R^{b} is independently halogen, cyano, C₁-C₄-alkyl, C₁-C₄-haloalkyl, C₁-C₄-alkoxy, and C₁-C₄-haloalkoxy; and
m is 1, 2, or 3;
R² is selected from the group consisting of phenyl which is unsubstituted or carries m radicals R^{b}, and 6- to 10-membered heteroaromatic ring containing 1 or 2 nitrogen atoms as ring members, which is unsubstituted or carries m radicals R^{b};
where
each R^{b} is independently selected from the group consisting halogen, nitro, C₁-C₄-alkyl, C₁-C₄-alkoxy, and phenyl; and
m is 1, 2, or 3;
R³ is hydrogen or C₁-C₄-alkyl;
or
—NR²R³ stands for 2,3-dihydroindol-1-yl, 1,2,3,4-tetrahydroquinolin-1-yl or indol-1-yl;
A is C₁-C₄-alkanediyl; and
R⁴ is C₁-C₄-alkyl or phenyl.

10. The method according to claim 1, where the compound of formula (II-1) and the compound of formula (III) are used in a molar ratio of from 5:1 to 1:5, and the compound of formula (II-2) and the compound of formula (III) are used in a molar ratio of from 2.5:1 to 1:10.

11. The method according to claim 1, where the Lewis acid is selected from the group consisting of the halides, nitrates, carboxylates where the anion has a formula R—COO⁻, where R is C₁-C₁₀-alkyl, C₃-C₆-cycloalkyl, or C₃-C₆-cycloalkyl-C₁-C₁₀-alkyl; acetylacetonates, C₁-C₄-alkoxides, and carbonyl complexes of metals of groups 4, 6 to 10, 12, 13, or 15 of the periodic table of elements.

12. The method according to claim 11, where the Lewis acid is selected from the group consisting of the halides, the carboxylates where the anion has the formula R—COO⁻, where R is C₁-C₄-alkyl, the carboxylates where the anion has the formula R—COO⁻, where R is C₃-C₆-cycloalkyl-C₁-C₁₀-alkyl, the acetylacetonates, the C₁-C₄-alkoxides and the carbonyl complexes of Mn, Co, Zn or Bi.

13. The method according to claim 11, where the Lewis acid is selected from the group consisting of MnCl₂, MoCl₃, CrCl₃, BiCl₃, SbCl₃, ZnCl₂, FeCl₃, FeCl₂, CoCl₂, NiCl₂, TiCl₄, ZrCl₄, HfCl₄, MnBr₂, Mn(NO₃)₂, Co(NO₃)₂, Mn(OAc)₂, Mn(4-cyclohexylbutyrat)₂, Fe(OAc)₃, Bi(OAc)₃, Mn(AcAc)₂, Mn(AcAc)₃, Fe(AcAc)₂, Fe(AcAc)₃, Ni(AcAc)₂, Bi(OiPr)₃, Ti(OiPr)₄, AI(OiPr)₃, Mn₂(CO)₁₀, Mn(CO)₅Br, Cr(CO)₆, Fe(CO)₄, and CO₂(CO)₈; where OAc means acetate, AcAc means acetylacetonate and OiPr means isopropoxide.

14. The method according to claim 13, where the Lewis acid is selected from the group consisting of MnCl₂, MnBr₂, BiCl₃, CoCl₂, ZnCl₂, Mn(OAc)₂, Mn(AcAc)₂, Mn(AcAc)₃, Mn(4-cyclohexylbutyrat)₂, Bi(OiPr)₃, Mn(CO)₅Br and Mn₂(CO)₁₀.

15. The method according to claim 1, where the Lewis acid is used in an amount of from 0.00001 to 0.2 mol.

16. The method according to claim 15, where the Lewis acid is used in an amount of from 0.001 to 0.01 mol per mol of compound (II) or (III) which is not used in excess.

17. The method according to claim 1, where the alkali metal-containing base is selected from the group consisting of alkali metal alkoxides, amides, hydrides, borohydrides and, aluminiumhydrides.

18. The method according to claim 1, where the alkali metal-containing base is used in an amount of from 0.00001 to 0.1 mol per mol of that compound (II) or (III) which is not used in excess.

19. The method according to claim 1, where
the Lewis acid is selected from the group consisting of the halides, the carboxylates where the anion has the formula R—COO⁻, where R is C₁-C₄-alkyl, the carboxylates where the anion has the formula R—COO⁻, where R is C₃-C₆-cycloalkyl-C₁-C₁₀-alkyl, the acetylacetonates, the C₁-C₄-alkoxides, and the carbonyl complexes of Mn, Co, Zn, or Bi; and
the alkali metal-containing base is selected from the group consisting of alkali metal C₁-C₁₀-alkoxides, alkali metal amides of the formula M⁺[N(R^{g})₂]⁻, where M⁺ is an alkali metal cation and R^{g} is hydrogen, C₁-C₄-alkyl, or Si(C₁-C₄-alkyl)₂; and alkali metal borohydrides of the formula M⁺[BH(C₁-C₄-alkyl)₃]⁻, where M⁺ is an alkali metal cation.

20. The method according to claim 1, where the water content in the reaction mixture is less than 0.1% by weight relative to the total weight of the reaction mixture.
